(19) United States
(12) Patent Application Publication
(10) Pub. No.: US 2022/0117968 A1
(43) Pub. Date: Apr. 21, 2022

(54) THIENO[2,3-D)PYRIMIDINES AND BENZOFURO(3,2-D)PYRIMIDINES AS ANTIMICROBIAL AGENTS

(71) Applicants: Saint Louis University, St. Louis, MO (US); Washington University, St. Louis, MO (US)

(72) Inventors: Marvin J. MEYERS, Wentzville, MO (US); Megh SINGH, Ellisville, MO (US); Christina L. STALLINGS, St. Louis, MO (US); Leslie A. WEISS, San Diego, CA (US); Scott WILDMAN, Madison, WI (US); Stacy D. ARNETT, St. Louis, MO (US)

(21) Appl. No.: 17/266,703

(22) PCT Filed: Jul. 17, 2018
(86) PCT No.: PCT/US2018/042425

§ 371 Date: Feb. 8, 2021

Related U.S. Application Data
(60) Provisional application No. 62/533,403, filed on Jul. 17, 2017.

(51) Int. Cl. A61K 31/519; C07D 495/04; C07D 491/048; A61K 31/4409; A61K 31/4965; A61K 31/496; A61K 31/7036; A61K 31/42; A61K 31/422; A61K 38/12; A61K 31/5383; A61K 31/4709; A61K 31/44; A61K 31/438; A61K 31/5377; A61K 31/175; A61K 31/5415; A61K 31/198; A61K 31/593; A61K 31/47; A61K 31/7048; A61K 31/133; A61P 31/06

(52) U.S. Cl. CPC A61K 31/519; A61P 31/06; C07D 491/048; A61K 31/4409; A61K 31/4965; A61K 31/496; A61K 31/7036; A61K 31/42; A61K 31/422; A61K 38/12; A61K 31/5383; A61K 31/4709; A61K 31/44; A61K 31/438; A61K 31/5377; A61K 31/175; A61K 31/5415; A61K 31/198; A61K 31/593; A61K 31/47; A61K 31/7048; A61K 31/133; C07D 495/04

(57) ABSTRACT

The present disclosure provides compounds, methods, and compositions which may be used to treat tuberculosis. In some embodiments, these compounds and compositions have a bactericidal property against Mycobacterium tuberculosis (Mtb). Methods of employing such agents are also provided.

This application claims the benefit of U.S. Provisional Application No. 62/533,403, filed on Jul. 17, 2017, the entire contents of which are hereby incorporated by reference.

This invention was made with government support under Grant No. R21/R33 AI111696 awarded by the National Institutes of Health and the National Institute of Allergy and Infectious Diseases. The government has certain rights in the invention.

### BACKGROUND

### I. Field of the Disclosure

The present disclosure relates to the fields of medicine, pharmacology and infectious disease. More particular, the disclosure relates to methods and compositions for treating tuberculosis.

### II. Related Art

Antibiotic resistant bacterial infections are a dangerous, worldwide health problem that requires costly and lengthy therapies that in many cases are ultimately ineffective. Infection with Mycobacterium tuberculosis (Mtb) results in over 9 million new cases of tuberculosis (TB) and 1.5 million deaths annually (World Health Organization Global Tuberculosis Report, 2015). A robust antibacterial defense usually controls primary Mtb infection by reducing bacterial numbers to uncultivable levels (Medlar, 1955) but is often unable to eradicate the pathogen, resulting in a large population of latently-infected individuals that may reactivate the infection later in life. In addition to its ability to resist elimination by host immunity, Mtb infection is only slowly sterilized by antibiotic treatment. Patients that are latently infected with Mtb require 3-9 months of antibiotic therapy to prevent reactivation of infection, despite low bacterial burdens. To achieve clinical cure in greater than 90% of patients with active TB, multidrug antibiotic therapy for 6 months is required. Because of the long courses of antibiotic therapy, incomplete therapy is common and has resulted in the rise of multidrug resistant (MDR) TB cases that are resistant to at least the two frontline antibiotics used to treat TB, isoniazid (INH) and rifampicin (RIF). MDR-TB constituted 3.7% of new TB cases in 2014 and 20% of previously treated TB cases, with rates of MDR-TB as high as 48% of TB cases in some countries (World Health Organization Global Tuberculosis Report, 2015). Furthermore, extensively drug resistant TB has now been isolated in almost 80 countries throughout the world, including the US. This rise in drug resistance and scarcity of drugs in the pipeline has made it clear that society are not equipped to successfully battle the TB epidemic. The inadequacies of present TB therapies demand the discovery of new agents with unique mechanisms of action to treat Mtb infection.

### SUMMARY

In some aspects, the present disclosure provides compounds which may be used in the treatment of tuberculosis or an infection of Mycobacterium tuberculosis. In some embodiments, the compounds are of the formula:

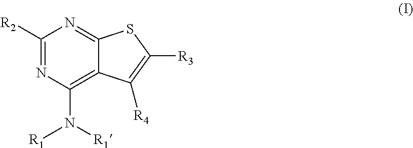

wherein:
R₁ is —(CH₂)ₓRₐ;
R₁′ is hydrogen, alkyl_{(C≤8)}, or substituted alkyl_{(C≤8)}, or —(CH₂)ₓRₐ, wherein:
   x is 3, 4, or 5;
   Rₐ is aryl_{(C≤12)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

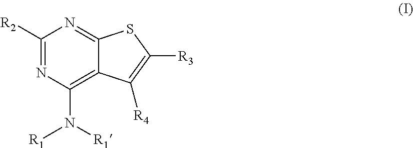

wherein:
R₁ is substituted aralkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

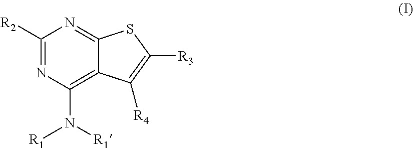

wherein:
R₁ and R₁′ are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is hydrogen, alkyl_{(C≤12)}, aryl_{(C≤12)}, heteroaryl_{(C≤12)}, aralkyl_{(C≤12)}, heteroaralkyl_{(C≤12)}, or a substituted version of the last five groups;
R₃ is halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is hydrogen, alkyl_{(C≤6)}, or substituted alkyl_{(C≤6)}; or
   a compound of the formula:

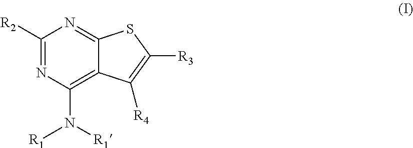

wherein:
R₁ is haloalkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

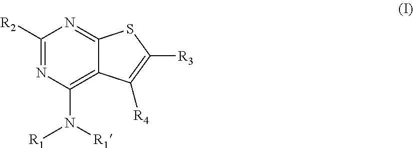

wherein:
R₁ is branched alkyl_{(C≤12)} or substituted branched alkyl_{(C≤8)};
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

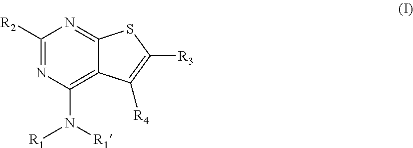

wherein:
R₁ and R₁′ is hydrogen, alkyl_{(C≤8)}, substituted alkyl_{(C≤8)}, aralkyl_{(C≤8)}, or substituted aralkyl_{(C≤8)};
R₂ is branched alkyl_{(C≤8)} or substituted branched alkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

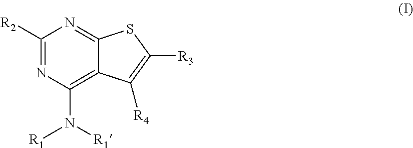

wherein:
R₁ and R₁′ is hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is haloalkyl_{(C≤8)} or substituted haloalkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
   a compound of the formula:

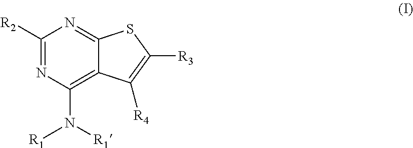

wherein:
R₁ and R₁′ are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is heteroaryl_{(C≤12)}, heteroaralkyl_{(C≤12)}, or a substituted version of either group;
R₃ is hydrogen, halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is hydrogen, alkyl_{(C≤6)}, or substituted alkyl_{(C≤6)}; or
   a compound of the formula:

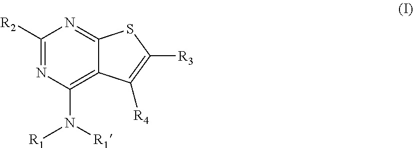

wherein:
R₁ and R₁′ are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is hydrogen, alkyl_{(C≤12)}, aryl_{(C≤12)}, heteroaryl_{(C≤12)}, aralkyl_{(C≤12)}, heteroaralkyl_{(C≤12)}, or a substituted version of the last five groups;
R₃ is hydrogen, halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is alkyl_{(C≤6)} or substituted alkyl_{(C≤6)}; or
   a compound of the formula:

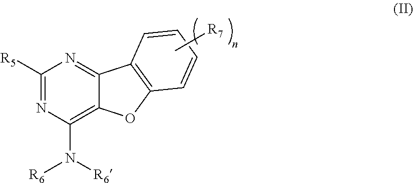

wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c}, wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
provided that when R₅ is methyl and n is 0, then R₆ is not butyl when R₆′ is hydrogen;
   or a pharmaceutically acceptable salt thereof. In some embodiments, the compounds are further defined as:

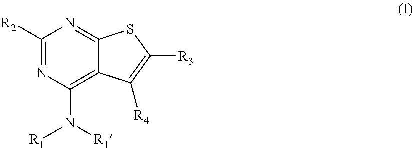

wherein:
R₁ is —(CH₂)ₓRₐ;
R₁′ is hydrogen, alkyl_{(C≤8)}, or substituted alkyl_{(C≤8)}, or —(CH₂)ₓRₐ, wherein:
   x is 3, 4, or 5;
   Rₐ is aryl_{(C≤12)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
   or a pharmaceutically acceptable salt thereof. In other embodiments, the compounds are further defined as:

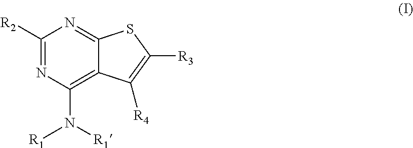

wherein:
R₁ is haloalkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
   or a pharmaceutically acceptable salt thereof. In other embodiments, the compounds are further defined as:

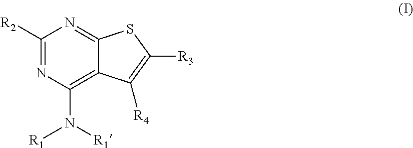

wherein:
R₁ and R₁′ is hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is haloalkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
   or a pharmaceutically acceptable salt thereof. In some embodiments, the compounds are further defined as:

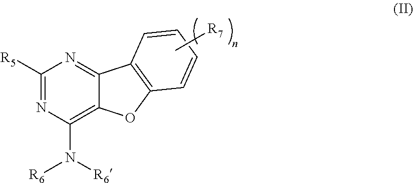

wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c} wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
provided that when R₅ is methyl and n is 0, then R₆ is not butyl when R₆′ is hydrogen;
   or a pharmaceutically acceptable salt thereof. In some embodiments, the compounds are further defined as:

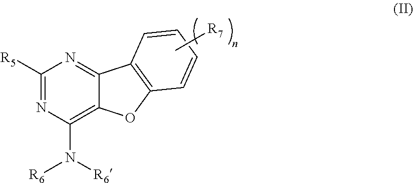

wherein:
R₅ is aryl_{(C≤12)}, aralkyl_{(C≤12)}, or a substituted version of either of these groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c} wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
   or a pharmaceutically acceptable salt thereof.

In some embodiments, R₂ is alkyl_{(C1-3)} such as methyl or ethyl. In other embodiments, R₂ is trifluoromethyl or pentafluoroethyl. In some embodiments, R₄ is hydrogen. In some embodiments, R₁ is hydrogen or methyl. In other embodiments, R₁ is halo. In some embodiments, R₁′ is 4,4,4-trifluorobutyl. In some embodiments, x is 3. In some embodiments, Rₐ is phenyl. In some embodiments, R₅ is alkyl_{(C1-3)} or substituted alkyl_{(C1-3)} such as methyl or ethyl. In some embodiments, n is 0. In some embodiments, R₆ is aralkyl_{(C≤12)} or substituted aralkyl_{(C≤12)} such as 3-phenylpropyl.

In some embodiments, the compounds are further defined as:

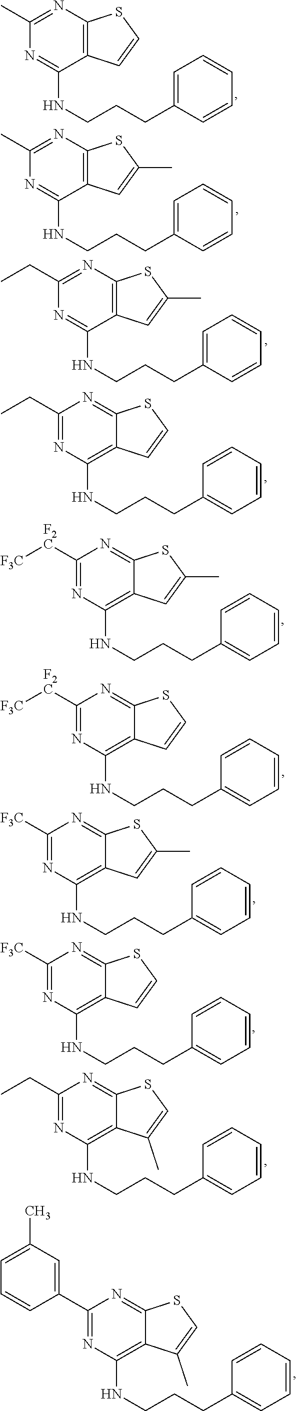

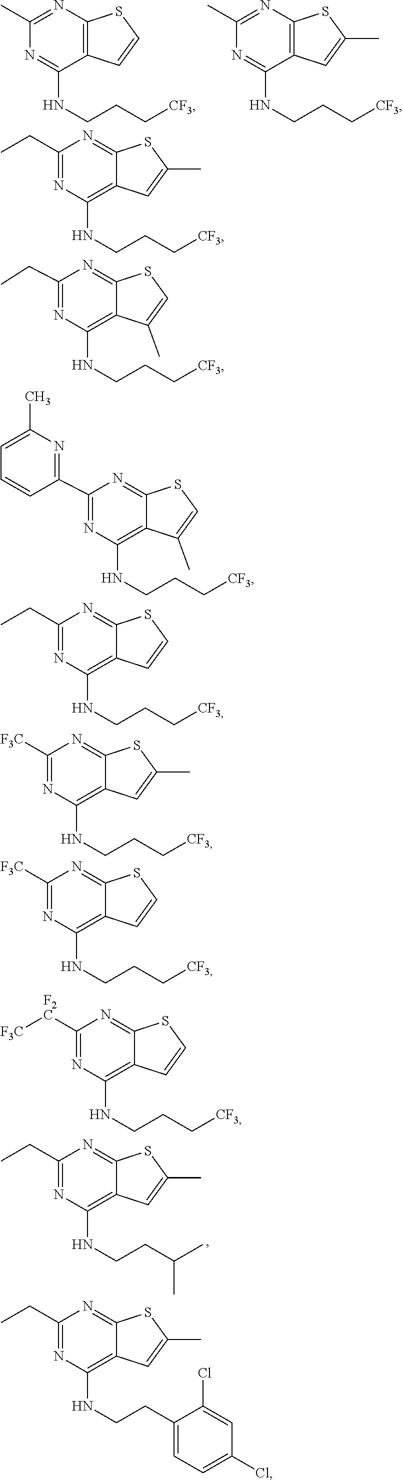

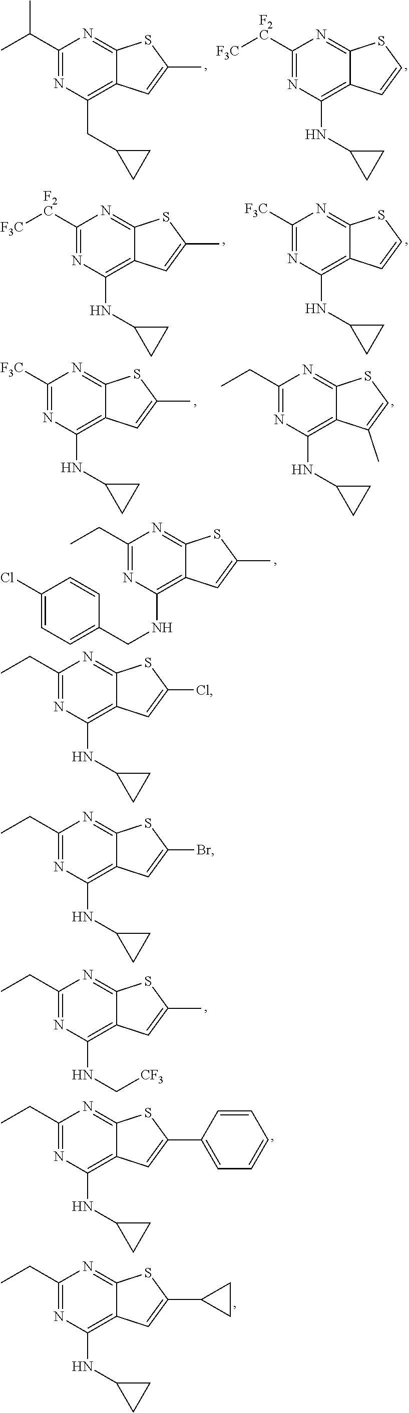

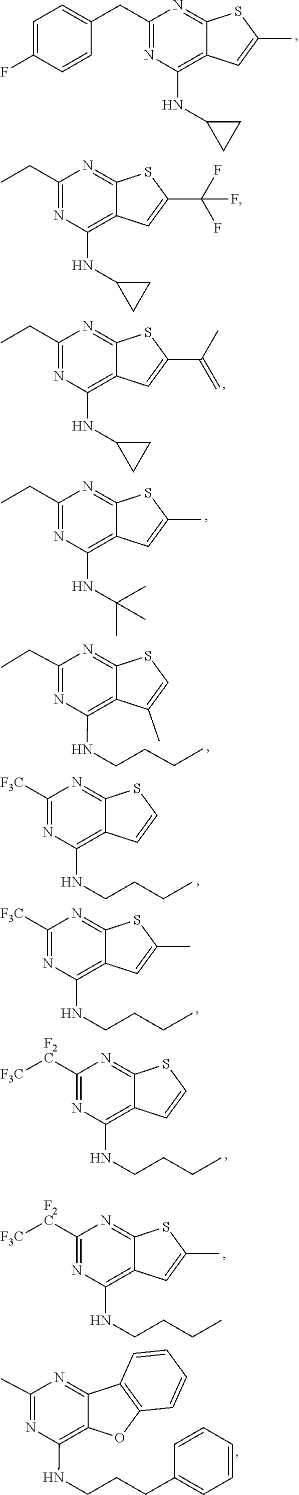

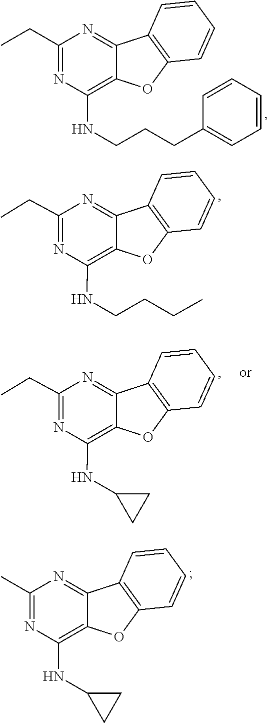

or a pharmaceutically acceptable salt thereof.

In another aspect, the present disclosure provides compounds of the formula:

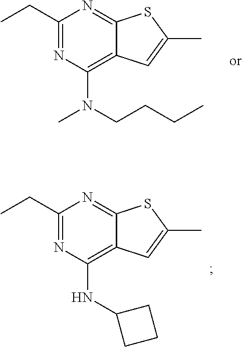

or a pharmaceutically acceptable salt thereof.

In yet another aspect, the present disclosure provides pharmaceutical compositions comprising:

(A) a compound described herein; and

(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In still yet another aspect, the present disclosure provides methods of treating tuberculosis in a patient comprising administering to the patient a therapeutically effective amount of a compound of the formula:

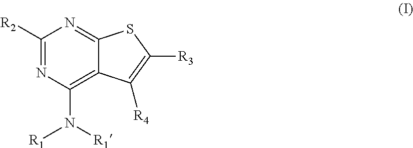

wherein:
R₁ is hydrogen, alkyl_{(C≤12)}, substituted alkyl_{(C≤12)}, cycloalkyl_{(C≤12)}, substituted cycloalkyl_{(C≤12)}, aralkyl_{(C≤12)}, or substituted aralkyl_{(C≤12)};
R₁′ is hydrogen or alkyl_{(C≤12)}, cycloalkyl_{(C≤12)}, aralkyl_{(C≤18)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these six groups;
R₃ is hydrogen, halo, or alkyl_{(C≤12)}, cycloalkyl_{(C≤12)}, alkenyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
   a compound of the formula:

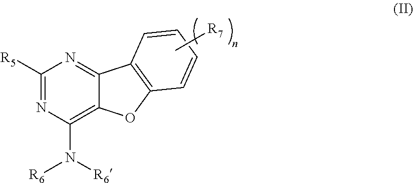

wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
   or a pharmaceutically acceptable salt thereof;
   provided that the compound is not:

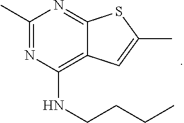 [SMILES: CCCCNc1nc(C)nc2sc(C)cc12]

In some embodiments, the compounds are further defined as a compound of formula I. In some embodiments, R₁ is hydrogen. In other embodiments, R₁′ is alkyl_{(C≤8)} or substituted alkyl_{(C≤8)}. In some embodiments, R₁′ is alkyl_{(C≤8)} such as n-butyl or 3-methylbutyl. In other embodiments, R₁′ is substituted alkyl_{(C≤8)} such as 4,4,4-trifluorobutyl. In other embodiments, R₁′ is cycloalkyl_{(C≤8)} or substituted cycloalkyl_{(C≤8)}. In some embodiments, R₁′ is cycloalkyl_{(C≤8)} such as cyclopropyl. In some embodiments, R₁′ is aralkyl_{(C≤12)} or substituted aralkyl_{(C≤12)}. In some embodiments, R₁′ is aralkyl_{(C≤12)} such as 3-phenylpropyl.

In some embodiments, R₂ is alkyl_{(C≤8)} such as methyl, ethyl, or isopropyl. In other embodiments, R₂ is fluoroalkyl_{(C≤8)} such as trifluoromethyl or pentafluoroethyl. In other embodiments, R₂ is aryl_{(C≤8)} such as phenyl. In other embodiments, R₂ is aralkyl_{(C≤8)} such as benzyl. In some embodiments, R₃ is hydrogen. In other embodiments, R₃ is halo such as chloro. In other embodiments, R₃ is alkyl_{(C≤8)} such as methyl. In some embodiments, R₄ is hydrogen.

In other embodiments, the compounds are further defined as a compound of formula II. In some embodiments, R₅ is alkyl_{(C≤8)} or substituted alkyl_{(C≤8)}. In some embodiments, R₅ is alkyl_{(C≤8)} such as methyl or ethyl. In some embodiments, R₆ is hydrogen. In other embodiments, R₆′ is alkyl_{(C≤8)} such as butyl. In other embodiments, R₆′ is cycloalkyl_{(C≤8)} such as cyclopropyl. In other embodiments, R₆′ is aralkyl_{(C≤8)} such as 3-phenylpropyl.

In some embodiments, the compounds are further defined as:

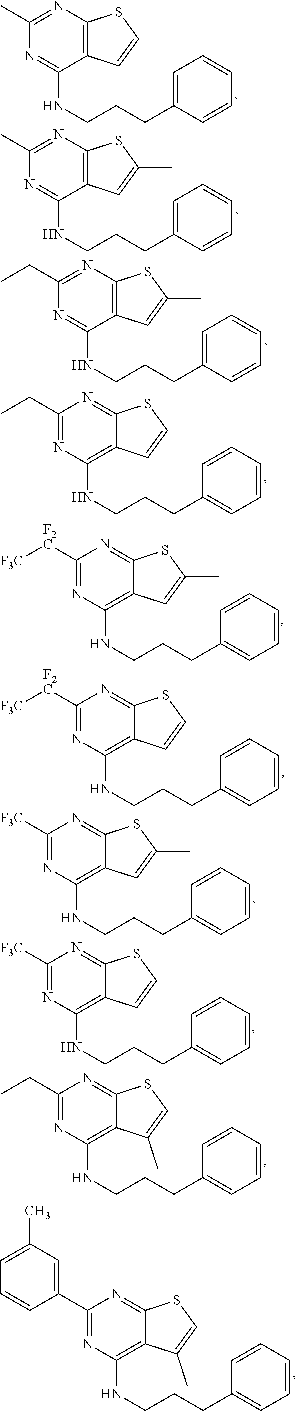

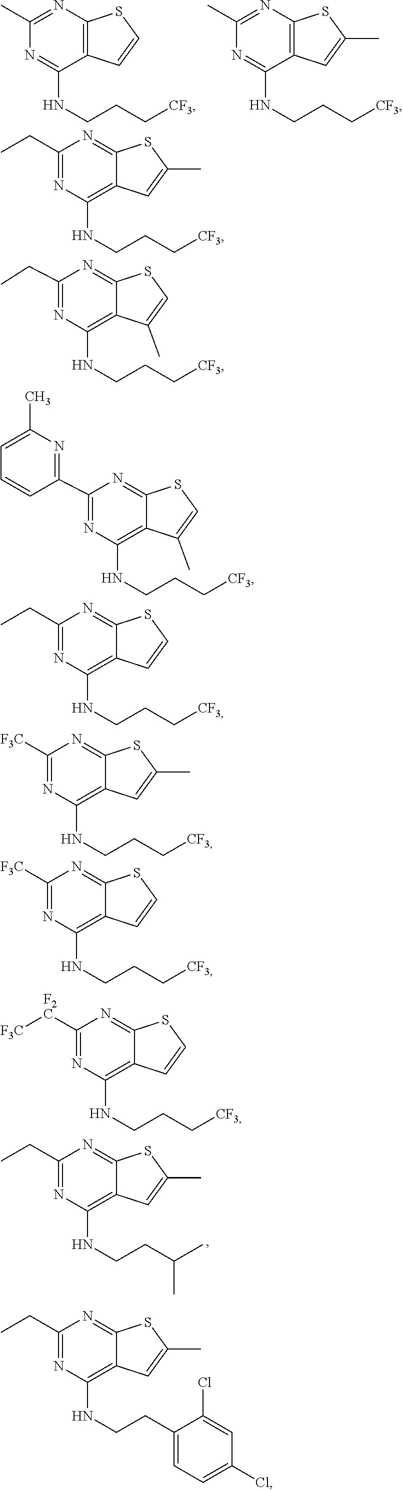

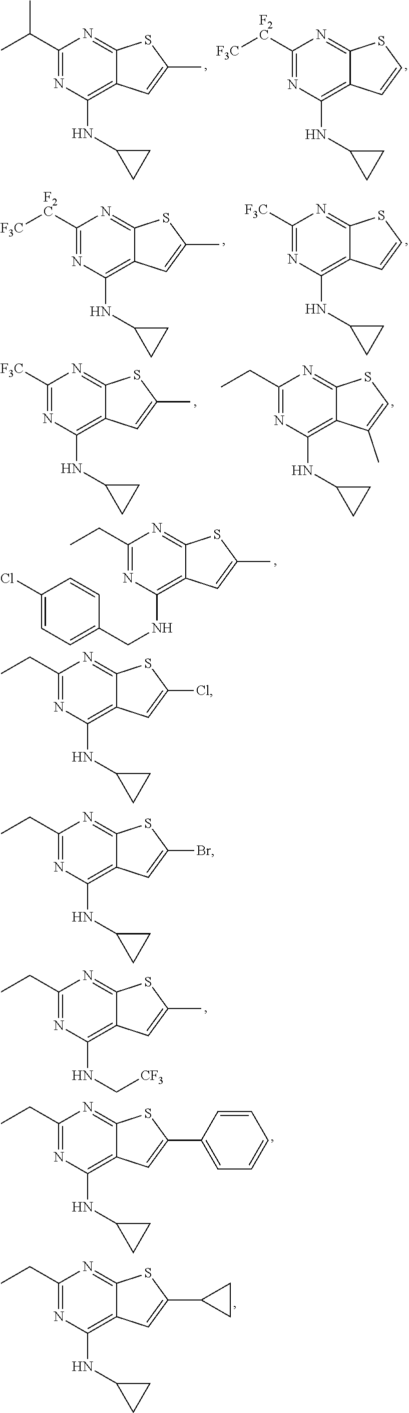

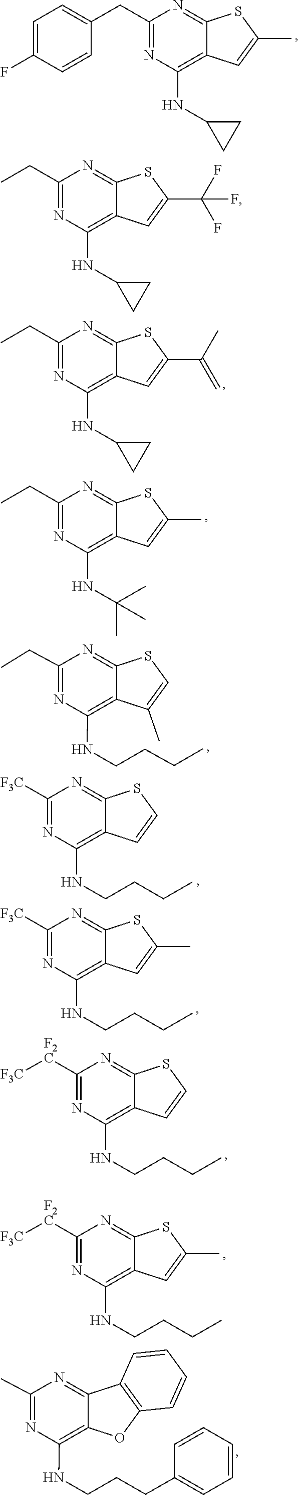

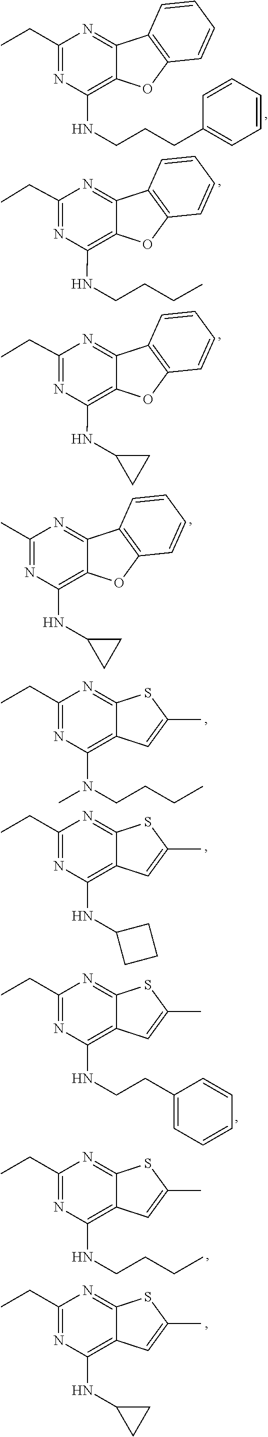

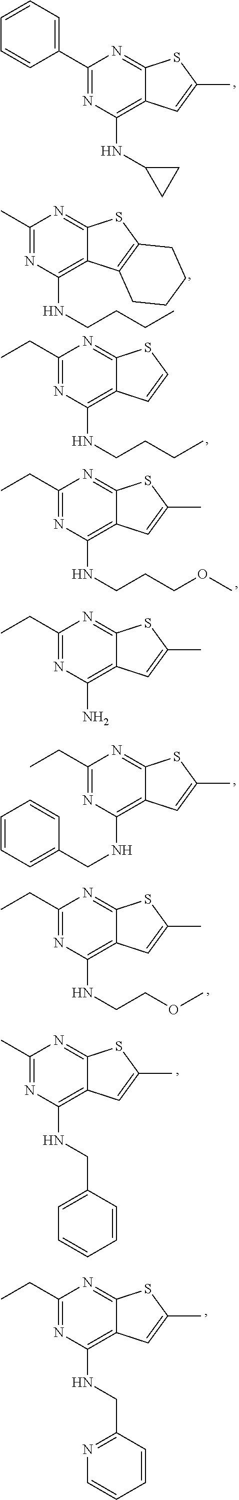

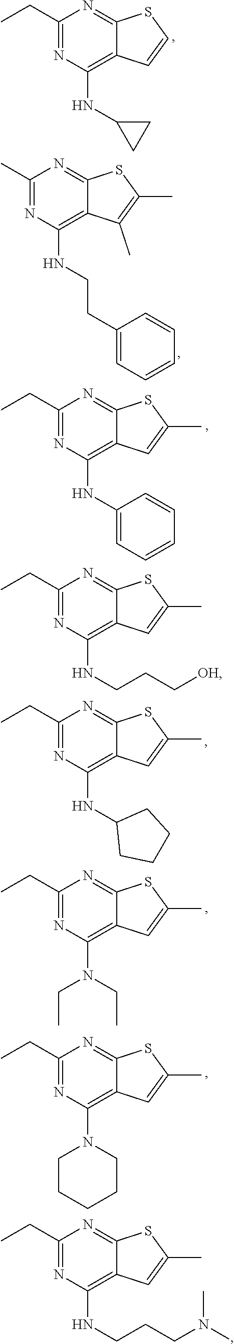

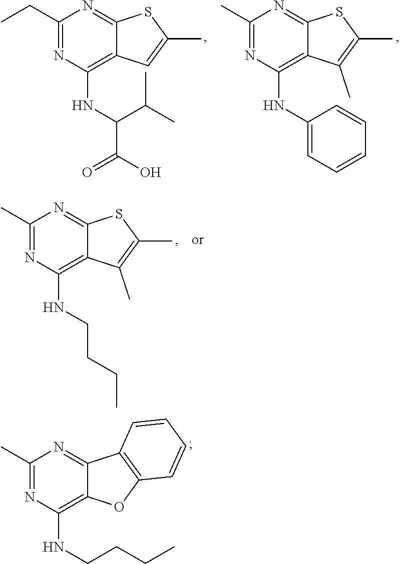

or a pharmaceutically acceptable salt thereof.

In some embodiments, the compounds are formulated as a pharmaceutical composition and further comprises an excipient. In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

In some embodiments, the tuberculosis is caused by a multi-drug resistant mycobacteria. The tuberculosis may be caused by a extensively drug resistant mycobacteria. In some embodiments, the patient is a mammal such as a human

The methods may further comprises a second anti-tuberculosis therapy such as a first line anti-tuberculosis therapy. In some embodiments, the first line anti-tuberculosis therapy is ethambutol, isoniazid, pyrazinamide, rifampicin, or streptomycin. In some embodiments, the second anti-tuberculosis therapy is a second line anti-tuberculosis therapy such as an aminoglycoside, a polypeptide antibiotic, a fluoroquinolone, a thioamide, cycloserine, or terizidone. In some embodiments, the aminoglycoside is amikacin or kanamycin. In some embodiments, the polypeptide antibiotic is capreomycin, viomycin, or enviomycin. In some embodiments, the fluoroquinolone is ciprofloxacin, levofloxacin, or moxifloxacin. In some embodiments, the thioamide is ethionamide or prothionamide.

In some embodiments, the second anti-tuberculosis therapy is a third line anti-tuberculosis therapy such as rifabutin, a macrolide, linezolid, thioacetazone, thioridazine, arginine, vitamin D, or bedaquiline. In some embodiments, the macrolide is clarithromycin. In some embodiments, the second anti-tuberculosis therapy further comprises 1, 2, 3, or 4 additional anti-tuberculosis therapies. In some embodiments, the methods further comprise administering the compound or pharmaceutical composition in combination with ethambutol, isoniazid, rifamycin, and pyrazinamide.

In some embodiments, the compound or the pharmaceutical composition is administered once. In some embodiments, the compound or the pharmaceutical composition is administered two or more times.

In still yet another aspect, the present disclosure provides methods of inducing the death of a Mycobacterium tuberculosis bacterium comprising contacting the bacteria with an effective amount of a compound of the formula:

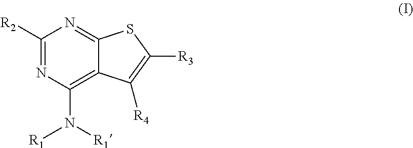

wherein:
R₁ is hydrogen, alkyl_{(C≤12)}, substituted alkyl_{(C≤12)}, cycloalkyl_{(C≤12)}, substituted cycloalkyl_{(C≤12)}, aralkyl_{(C≤12)}, or substituted aralkyl_{(C≤12)};
R₁′ is hydrogen or alkyl_{(C≤12)}, cycloalkyl_{(C≤12)}, aralkyl_{(C≤18)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these six groups;
R₃ is hydrogen, halo, or alkyl_{(C≤12)}, cycloalkyl_{(C≤12)}, alkenyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
   a compound of the formula:

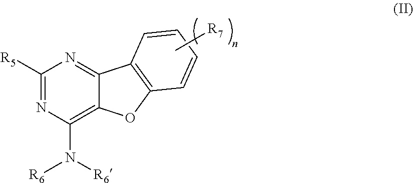

wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
   or a pharmaceutically acceptable salt thereof;
   provided that the compound is not:

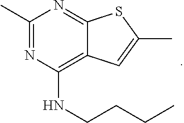 [SMILES: CCCCNc1nc(C)nc2sc(C)cc12]

In some embodiments, the compounds are further defined as:

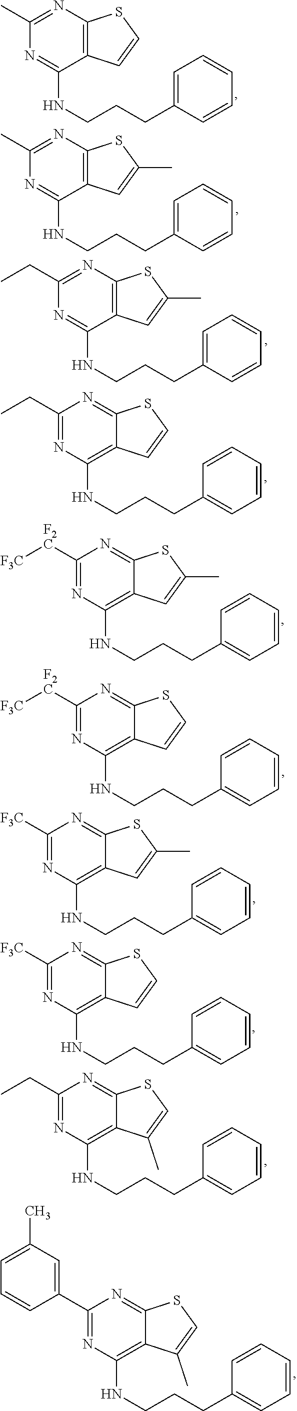

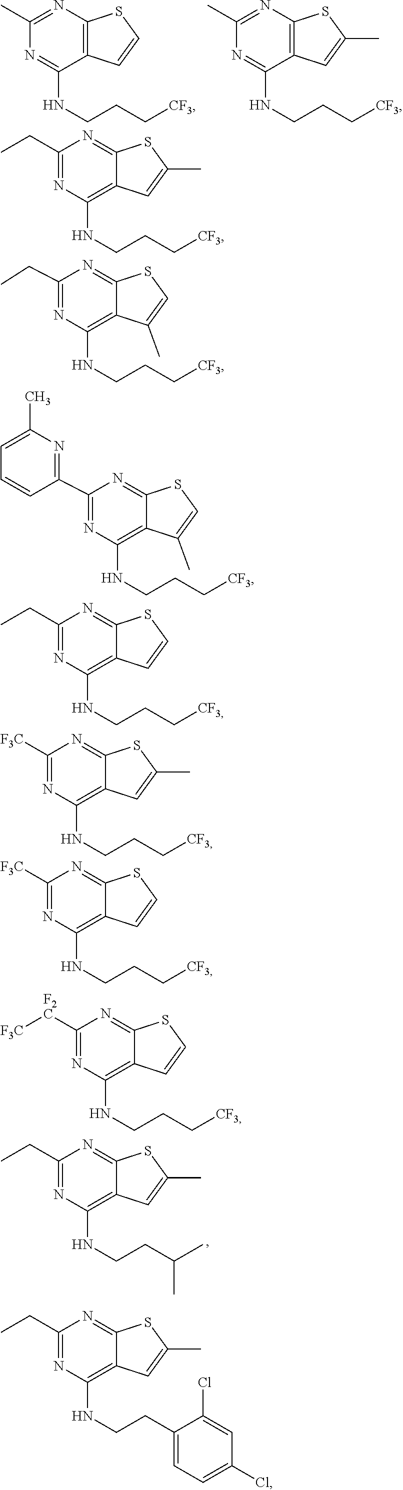

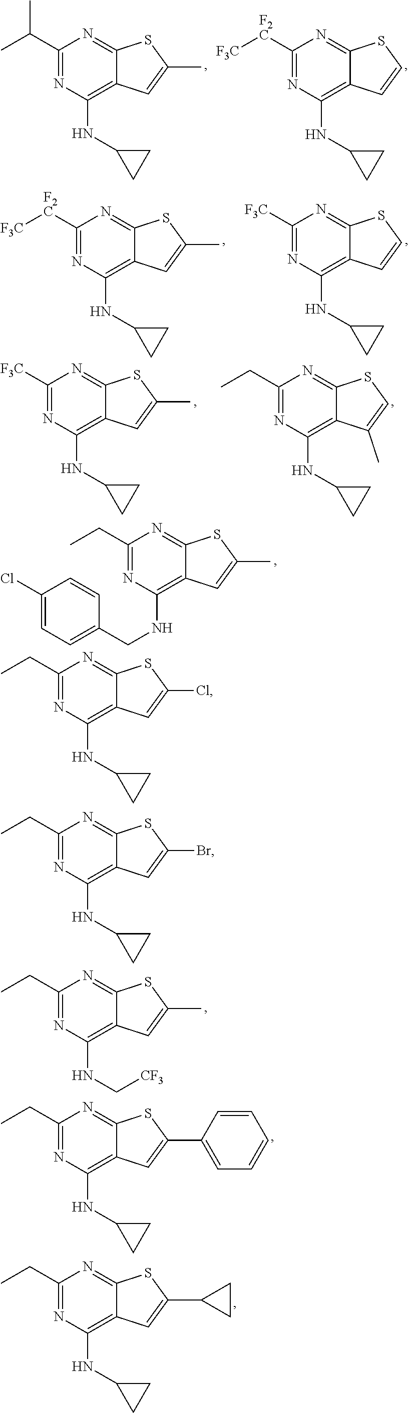

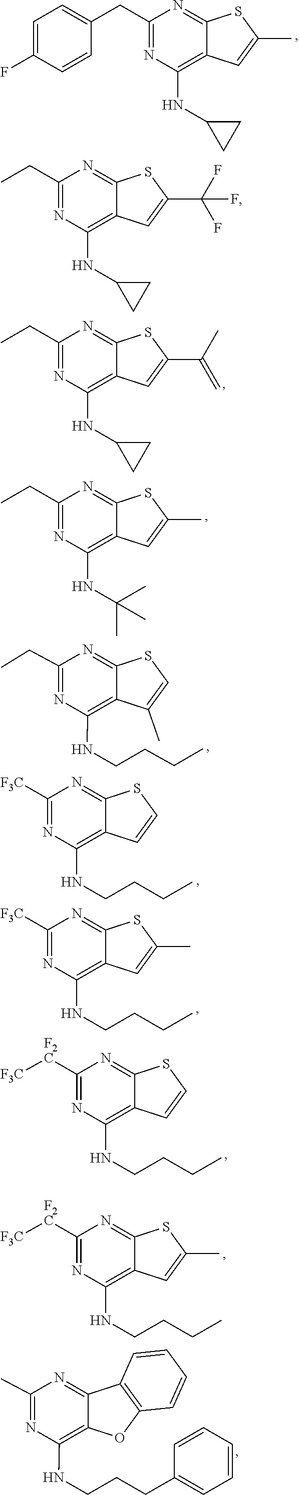

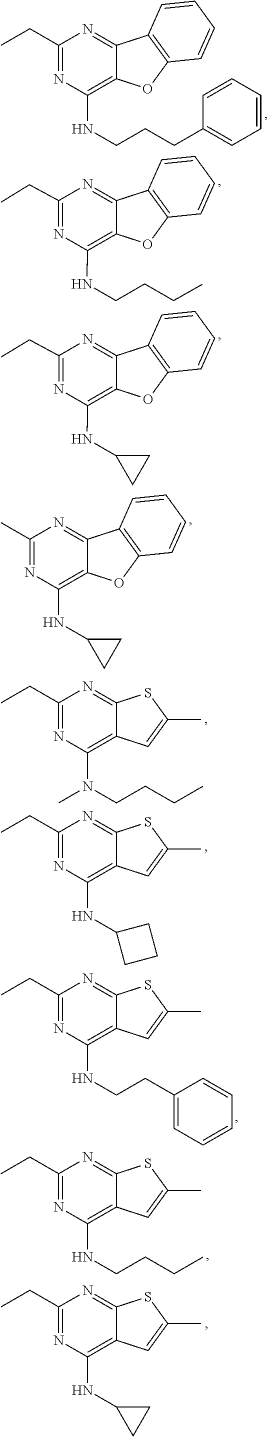

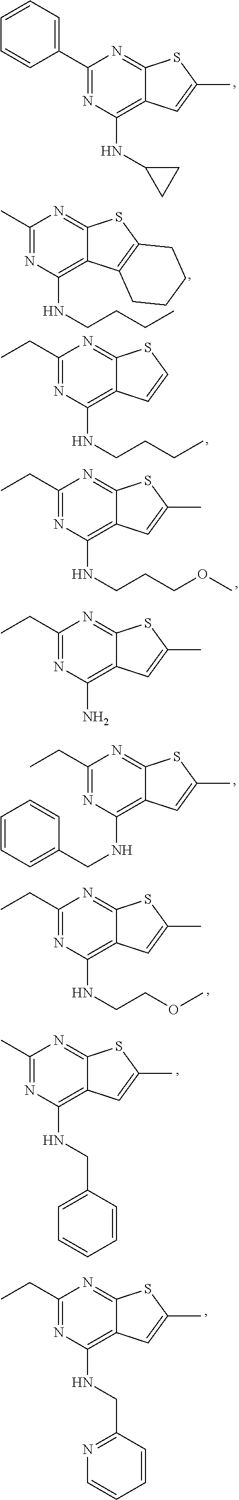

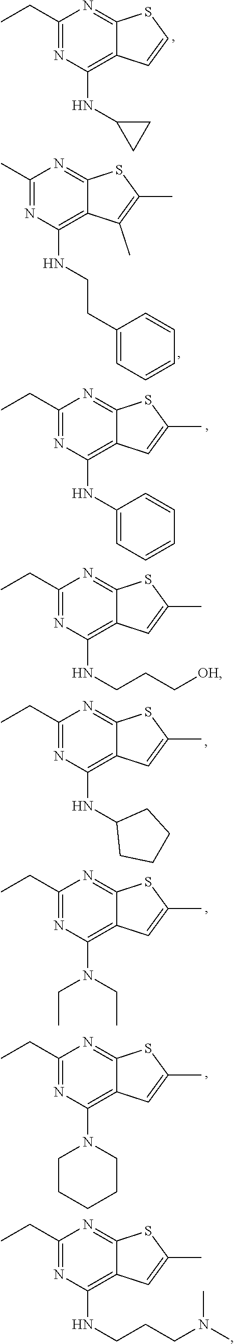

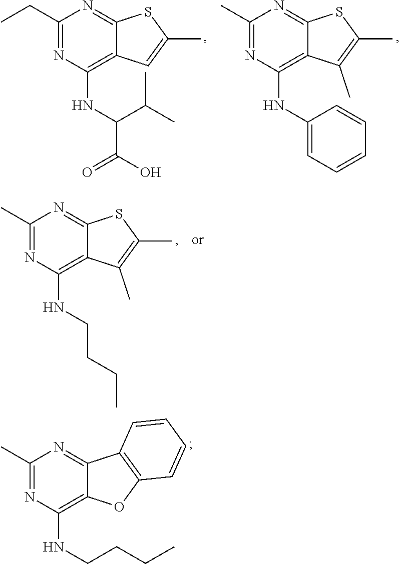

or a pharmaceutically acceptable salt thereof. In some embodiments, the methods are sufficient to treat a Mycobacterium tuberculosis infection in a patient.

In yet another aspect, the present disclosure provides methods of inhibiting the replication of a Mycobacterium tuberculosis bacterium comprising contacting the bacteria with an effective amount of a compound of the formula:

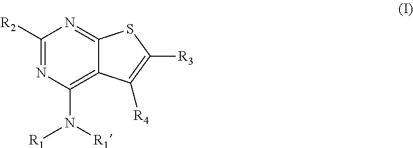

wherein:
R₁ is hydrogen, alkyl_{(C≤12)}, substituted alkyl_{(C≤12)}, cycloalkyl_{(C≤12)}, or substituted cycloalkyl_{(C≤12)};
R₁′ is hydrogen or alkyl_{(C≤12)}, cycloalkyl_{(C≤12)}, aralkyl_{(C≤12)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, aralkyl_{(C≤12)}, or a substituted version of any of these four groups;
R₃ is hydrogen, halo, or alkyl_{(C≤12)}, cycloalkyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
   a compound of the formula:

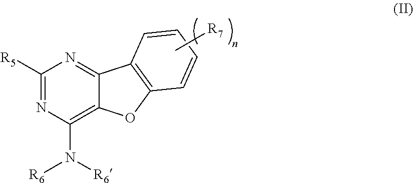

wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
   or a pharmaceutically acceptable salt thereof;
   provided that the compound is not:

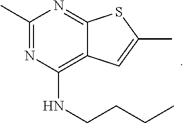 [SMILES: CCCCNc1nc(C)nc2sc(C)cc12]

In some embodiments, the compounds are further defined as:

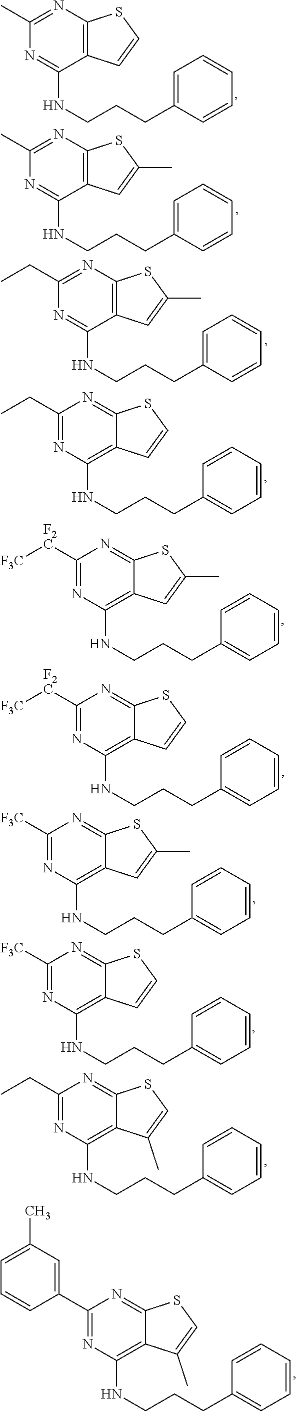

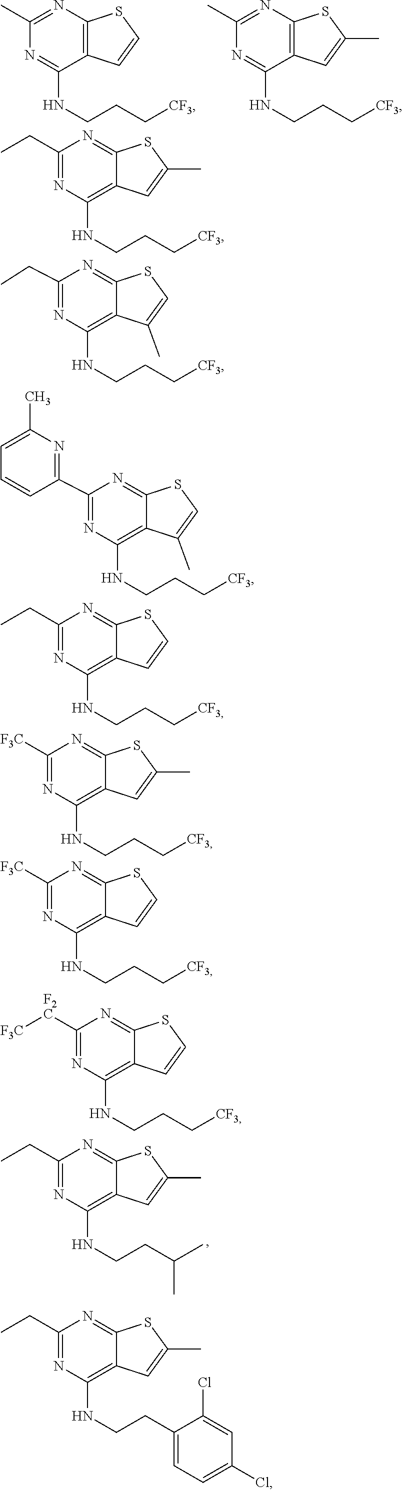

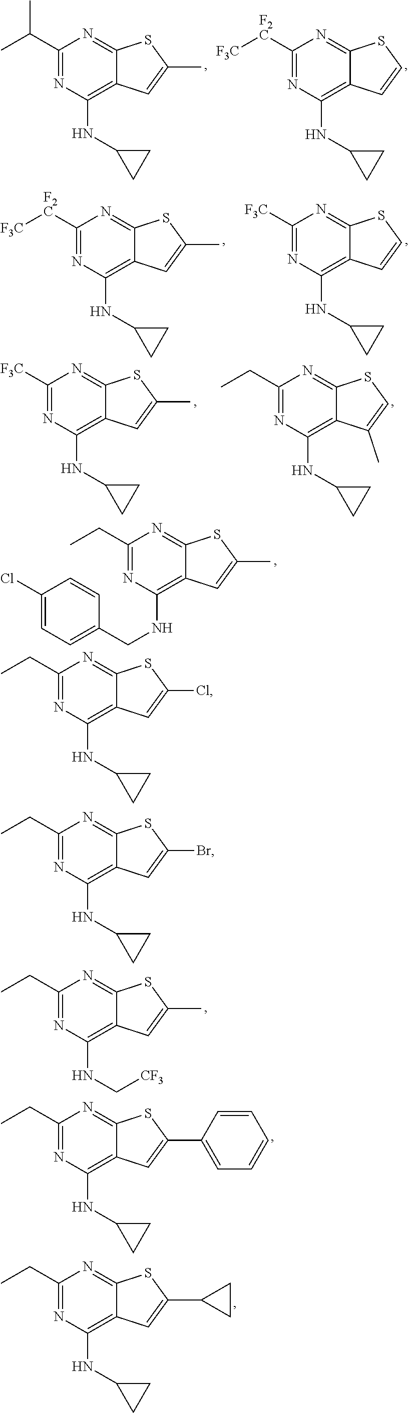

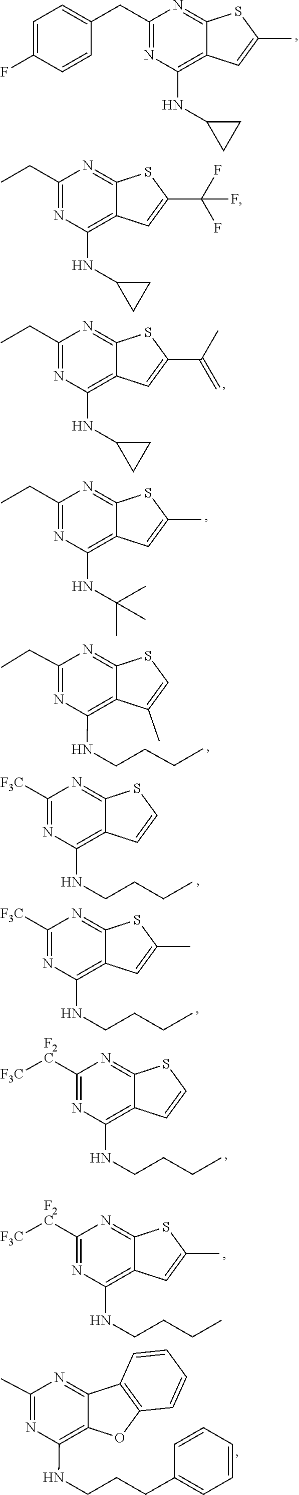

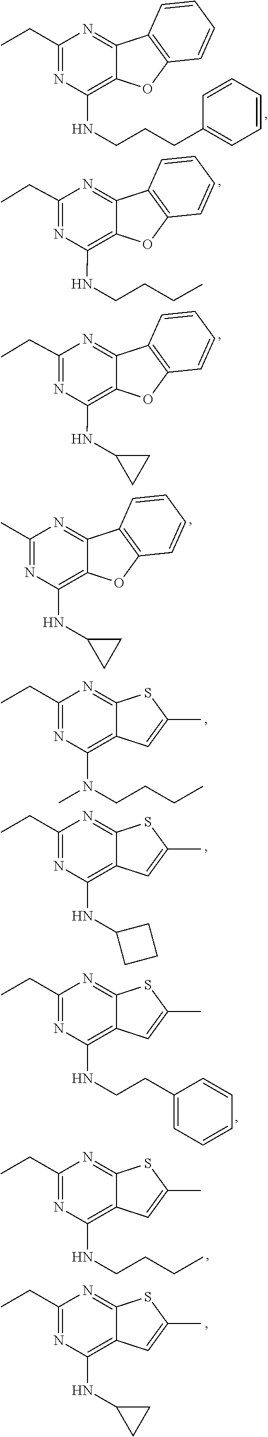

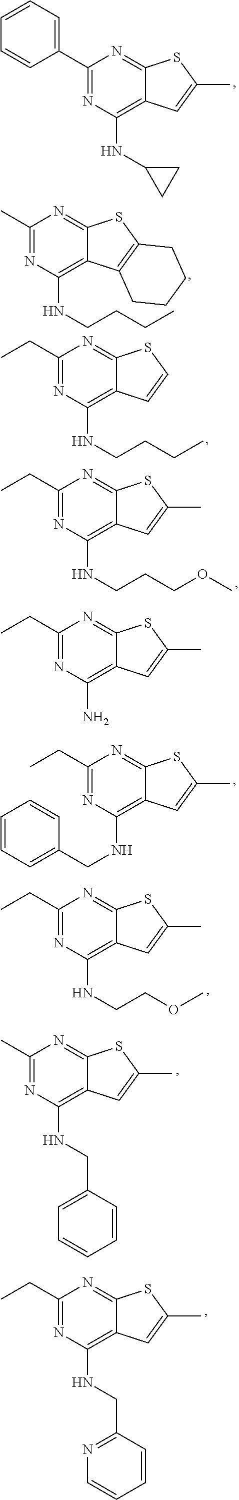

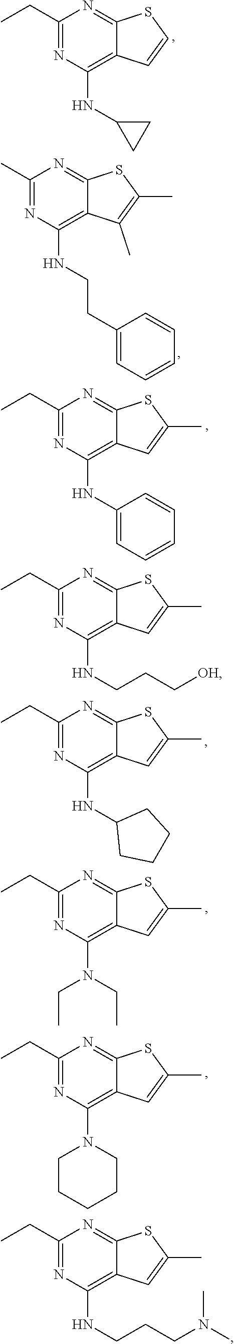

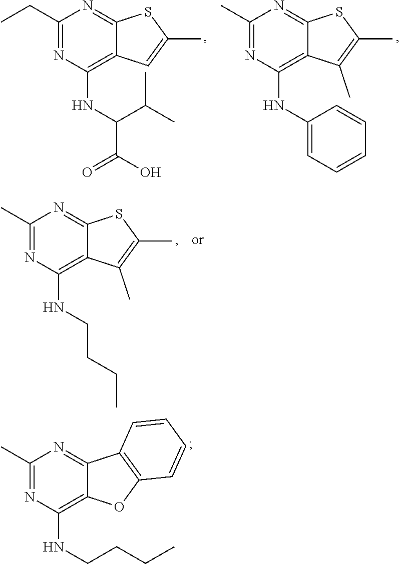

or a pharmaceutically acceptable salt thereof.

In some embodiments, the methods are sufficient to treat a Mycobacterium tuberculosis infection in a patient.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### DETAILED DESCRIPTION OF THE PREFERRED EMBODIMENTS

The present disclosure provides compounds which are useful for the treatment of tuberculosis and other diseases such as infections caused by Mycobacterium tuberculosis. In some embodiments, the compounds provided are used to treat tuberculosis. In some embodiments, the Mycobacterium tuberculosis may be a drug resistant Mycobacterium tuberculosis which is resistant to one or more of the front line antibiotic drugs such as isoniazid and rifampicin.

### I. TUBERCULOSIS

Tuberculosis is a disease caused by an infection of Mycobacterium tuberculosis. Generally, this bacterium infects the lungs and results in a latent infection in which no discernable symptoms can be detected. In some cases, the latent condition can progress into the active form of the disease. In some estimates, infection with Mtb results in over 9 million new cases of TB and 1.5 million deaths annually (World Health Organization Global Tuberculosis Report, 2015). Some estimates have contemplated that at least a third of the world population is infected with Mtb. Symptoms of an active infection include a chronic cough often associated with blood-containing sputum, fever, night sweats, and weight loss. The bacterium is transmitted through the air from patients with an active infection, while patients with a latent infection are generally not contagious.

Subjects with weakened immune system such as those with HIV/AIDS or who smoke, subjects who work in high risk environments such as hospitals, schools, or house with a person with an active infection are at high risk of contracting TB. Diagnosis occurs through the use of a latent testing protocol such as a skin test or an interferon gamma release assay but these particular tests are not useful to identifying an active infection and rather are only used to determine the presence of a latent infection. Active infections are often identified by the use of a chest X-ray or sputum cultures for acid-fast bacteria. The standard for determining the presence of an active infection though is the detection of Mtb in a clinical sample such as sputum or tissue.

Treatment of TB involves administering to the patient a sufficient amount of a therapeutic agent such as an antibiotic. A robust antibacterial defense usually controls primary Mtb infection by reducing bacterial numbers to uncultivable levels (Medlar, 1955) but is often unable to eradicate the pathogen, resulting in a large population of latently-infected individuals that may reactivate the infection later in life. In addition to its ability to resist elimination by host immunity, Mtb infection is only slowly sterilized by antibiotic treatment. Patients that are latently infected with Mtb require 3-9 months of antibiotic therapy to prevent reactivation of infection, despite low bacterial burdens. Typical first generation treatment includes the use of a cocktail of agents. including isoniazid, rifampicin, pyrazinamide, and ethambutol. This particular cocktail is often used in four significant courses including using the four drug combination daily or at least five times a week for 8 weeks and followed by a course of isoniazid and rifampicin daily or at least five times a week for 18 weeks, using the four drug combination daily or at least five times a week for 8 weeks and followed by a course of isoniazid and rifampicin three times a week for 18 weeks, using the four drug combination three times a week for 8 weeks and followed by a course of isoniazid and rifampicin three times a week for 18 weeks, or using the four drug combination daily for 2 weeks, followed by 2 days a week for 6 weeks, and followed by a course of isoniazid and rifampicin twice a week for 18 weeks. To achieve clinical cure in greater than 90% of patients with active TB, multidrug antibiotic therapy for 6 months is required. Because of the long courses of antibiotic therapy, incomplete therapy is common and has resulted in the rise of multidrug resistant (MDR) TB cases that are resistant to at least the two frontline antibiotics used to treat TB, isoniazid (INH) and rifampicin (RIF). MDR-TB constituted 3.7% of new TB cases in 2014 and 20% of previously treated TB cases, with rates of MDR-TB as high as 48% of TB cases in some countries (World Health Organization Global Tuberculosis Report, 2015). Furthermore, extensively drug resistant TB has now been isolated in almost 80 countries throughout the world, including the US. In cases of multidrug resistant tuberculosis or other difficult to treat cases of tuberculosis, an additional agent maybe used. These agents are often divided into four different groups: Group A of fluoroquinolones including levofloxacin, moxifloxacin, or gatifloxacin, Group B of injectable anti-TB drugs including kanamycin, amikacin, streptomycin, or capreomycin, Group C of second-line agents including ethionamide, prothionamide, cycloserine, terizidone, linezolid, or clofazinime, and Group D of add-on agents including high-dose isoniazid, pyrazinamide, ethambutol, bedaquiline, delamanid, para-aminosalicylci acid, imipenem with either cilastatin or meropenem with clavulanate, or thiocetazone. Additionally, a vaccine such as the BCG vaccine may be administered in cases to prevent active infections.

### II. ACTIVE AGENTS AND INTERMEDIATES

A. Compounds of the Present Disclosure

The compounds of the present disclosure are shown, for example, above, in the summary of the disclosure section and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch of continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All of the compounds of the present disclosure may be useful for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all of the compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atoms, and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present disclosure.

B. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

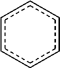

covers, for example,

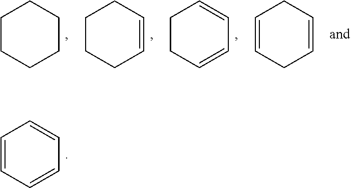

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond

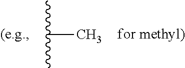

indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

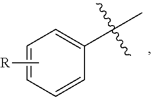

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

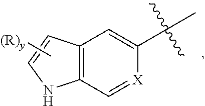

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in the moiety replacing a hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic π system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

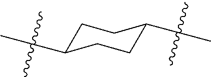 [SMILES: *C1CCC(*)CC1]

is a non-limiting example of cycloalkanediyl group. A “cycloalkane” refers to the class of compounds having the formula H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkenyl” when used without the “substituted” modifier refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —CH═CHF, —CH═CHCl and —CH═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aryl” when used without the “substituted” modifier refers to a monovalent unsaturated aromatic group with an aromatic carbon atom as the point of attachment, said carbon atom forming part of a one or more aromatic ring structure, wherein the ring atoms are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

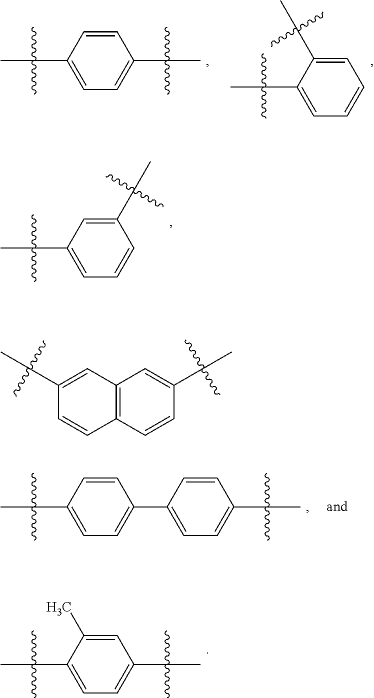

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group-alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. Unfused rings are connected with a covalent bond. As used herein, the term heteroaryl does not preclude the presence of one or more alkyl or aryl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects.

An “active ingredient” (AI) (also referred to as an active compound, active substance, active agent, pharmaceutical agent, agent, biologically active molecule, or a therapeutic compound) is the ingredient in a pharmaceutical drug or a pesticide that is biologically active. The similar terms active pharmaceutical ingredient (API) and bulk active are also used in medicine, and the term active substance may be used for pesticide formulations.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical agent, pharmaceutical preparation, pharmaceutical composition, pharmaceutical formulation, pharmaceutical product, medicinal product, medicine, medication, medicament, or simply a drug) is a drug used to diagnose, cure, treat, or prevent disease. An active ingredient (AI) (defined above) is the ingredient in a pharmaceutical drug or a pesticide that is biologically active. The similar terms active pharmaceutical ingredient (API) and bulk active are also used in medicine, and the term active substance may be used for pesticide formulations. Some medications and pesticide products may contain more than one active ingredient. In contrast with the active ingredients, the inactive ingredients are usually called excipients (defined above) in pharmaceutical contexts.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Substituent convertible to hydrogen in vivo” means any group that is convertible to a hydrogen atom by enzymological or chemical means including, but not limited to, hydrolysis and hydrogenolysis. Examples include hydrolyzable groups, such as acyl groups, groups having an oxycarbonyl group, amino acid residues, peptide residues, o-nitrophenylsulfenyl, trimethylsilyl, tetrahydropyranyl, diphenylphosphinyl, and the like. Examples of acyl groups include formyl, acetyl, trifluoroacetyl, and the like. Examples of groups having an oxycarbonyl group include ethoxycarbonyl, tert-butoxycarbonyl (—C(O)OC(CH₃)₃), benzyloxycarbonyl, p-methoxybenzyloxycarbonyl, vinyloxycarbonyl, β-(p-toluenesulfonyl)ethoxycarbonyl, and the like. Suitable amino acid residues include, but are not limited to, residues of Gly (glycine), Ala (alanine), Arg (arginine), Asn (asparagine), Asp (aspartic acid), Cys (cysteine), Glu (glutamic acid), His (histidine), Ile (isoleucine), Leu (leucine), Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable amino acid residues also include amino acid residues that are protected with a protecting group. Examples of suitable protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Suitable peptide residues include peptide residues comprising two to five amino acid residues. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethoxycarbonyl groups (such as benzyloxycarbonyl and p-nitrobenzyloxycarbonyl), tert-butoxycarbonyl groups (—C(O)OC(CH₃)₃), and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and haloethoxycarbonyl groups (such as β,β,β-trichloroethoxycarbonyl and β-iodoethoxycarbonyl).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### III. THERAPEUTIC METHODS

A. Pharmaceutical Formulations and Routes of Administration

For the purpose of administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound of the present disclosure formulated with one or more excipients and/or drug carriers appropriate to the indicated route of administration. In some embodiments, the compounds of the present disclosure are formulated in a manner amenable for the treatment of human and/or veterinary patients. In some embodiments, formulation comprises admixing or combining one or more of the compounds of the present disclosure with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. Pharmaceutical formulations may be subjected to conventional pharmaceutical operations, such as sterilization and/or may contain drug carriers and/or excipients such as preservatives, stabilizers, wetting agents, emulsifiers, encapsulating agents such as lipids, dendrimers, polymers, proteins such as albumin, or nucleic acids, and buffers, etc.

Pharmaceutical formulations may be administered by a variety of methods, e.g., orally or by injection (e.g. subcutaneous, intravenous, intraperitoneal, etc.). Depending on the route of administration, the compounds of the present disclosure may be coated in a material to protect the compound from the action of acids and other natural conditions which may inactivate the compound. To administer the active compound by other than parenteral administration, it may be necessary to coat the compound with, or co-administer the compound with, a material to prevent its inactivation. For example, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds of the present disclosure may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds of the present disclosure can be administered orally, for example, with an inert diluent or an assimilable edible carrier. The compounds and other ingredients may also be enclosed in a hard or soft shell gelatin capsule, compressed into tablets, or incorporated directly into the subject's diet. For oral therapeutic administration, the compounds of the present disclosure may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

In some embodiments, the therapeutic compound may also be administered topically to the skin, eye, or mucosa. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. In some embodiments, the specification for the dosage unit forms of the disclosure are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In general a human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose(mg/kg)×(Animal Kₘ/Human Kₘ)

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat.

B. Combination Therapy

In addition to being used as a monotherapy, the compounds of the present disclosure may also find use in combination therapies. Effective combination therapy may be achieved with a single composition or pharmacological formulation that includes both agents, or with two distinct compositions or formulations, administered at the same time, wherein one composition includes a compound of this disclosure, and the other includes the second agent(s). Alternatively, the therapy may precede or follow the other agent treatment by intervals ranging from minutes to months.

It is contemplated that any antibiotic may be administered in combination with the compounds of the present disclosure in order to treat a TB infection. In some cases, the TB infection may be a drug resistant strain which may be treated with a combination of multiple antibiotics. Some exemplary antibiotics an other therapeutic agents include isoniazid, pyrazinamide, rifampicin, ethambutol, levofloxacin, moxifloxacin, gatifloxacin, kanamycin, amikacin, capreomycin, streptomycin, ethionamide, prothionamide, cycloserine, terizidone, linezolid, clofazimine, bedaquiline, delamanid, para-aminosalicylic acid, imipenem, cilastatin, meropenem, or thiocetazone. In particular embodiments, the combination methods may comprise treating with one or more of rifampicin, pyrazinamide, ethambutol, and isoniazid. In some embodiments, a therapy may comprise all four of these antibiotics. Additionally, if resistance to one of these two antibiotics is detected, then bedaquiline or linezolid may also be administered instead of one or the above noted antibiotics.

Finally, given the difficulty in treating TB infections, such combination therapies may be used for multiple months. Extremely resistant TB infections may be treated for 1 to 3 years in order to completely rid the body of the Mycobacterium tuberculosis bacterium completely. For less extensive or less difficult bacterial strains to treat, the treatments may also from 3 to 12 months instead of 1 to 3 years.

### IV. EXAMPLES

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### A. General Methods

Instrumentation and General Methods. Analytical HPLC analyses were performed on an Agilent 1100 system and LC-MS analyses were conducted on Agilent 1100 Series LC/MSD (G1946C) single quadrupole mass spectrometer system equipped with an electrospray ionization (ESI) source. Reverse-phase preparative HPLC purifications were performed either on a Biotage SP4 HPFC system or on a CombiFlashRf (Teledyne Isco) system using a variable dual wavelength UV detector on a Biotage KP-C18-HS 120 g SNAP column and on Redisep Rf Gold C18 cartridges using acetonitrile/water gradient containing 0.05% TFA. Normal phase preparative HPLC purifications were performed either on a Biotage SP4 HPFC system or on a CombiFlashRf (Teledyne Isco) system using a variable dual wavelength UV detector with pre-packed Biotage KP-SIL SNAP cartridges and Redisep Rf silica gel (Isco) cartridges and ethyl acetate/hexanes gradients.

All final compounds were analyzed by analytical HPLC using a C18 analytical column with a diode array detector and peaks were monitored at 210, 254 and 280 nM for their purity. ¹H and ¹⁹F NMR spectra were recorded in deuterated solvents (DMSO-d₆, CD₃OD and CDCl₃) on a Bruker Avance-III/400 MHz spectrometer equipped with a Broad Band NMR probe. The signal of the deuterated solvent was used as an internal reference. The chemical shifts are expressed in ppm (δ) and coupling constants (J) are reported in hertz (Hz). Reactions were performed under an atmosphere of dry nitrogen unless otherwise stated.

The starting materials were obtained from commercial sources and used without further purification after verifying their purities by LC-MS analysis. Solvents were analytical grade and used as supplied. Non-commercially available starting materials were synthesized following the literature procedures and used after further purification and verifying their purities by ¹H NMR and LC-MS analysis.

MABA Assay. The compounds have been evaluated in the Microplate Alamar Blue Assay (MABA), which is commonly used to evaluate the efficacy of compounds in restraining Mtb growth (Franzblau, 1992). The MABA utilizes the dye resazurin, which is dark blue and nonfluorescent in its oxidized form but becomes pink and fluorescent when reduced to resorufin as a result of cellular metabolism. The degree of this color change is monitored and quantified, and compounds that inhibit Mtb growth or survival will decrease or block this color change. By performing this assay with WT Mtb Erdman strain in Middlebrook 7H9 media (Sigma-Aldrich) with a range of concentrations of each compound, we are able to calculate MIC₅₀ and MIC₉₀ values for active compounds, which is defined as the concentration of antibiotic that inhibits mycobacterial survival by 50% and 90%, respectively. These assays were performed in 96 well dishes and the compounds were added to the cultures at the time of Mtb inoculation and incubated for 7 days at 37° C. at 5% CO₂, at which point the resazurin dye was added for 24 hours before measuring the fluorescence at excitation 530 nm and emission 590 nm. Disk Zone of Inhibition Assay. A disk zone of inhibition assay was also used to assess the activity of compounds in WT Mtb Erdman. In these assays, 5 μl of a 100 mM stock of compound dissolved in DMSO is spotted on a disk in the center of a lawn of bacteria on Middlebrook 7H10 agar media. After incubation at 37° C. in 5% CO₂ for 10 days, the radius of the zone absent of bacterial growth is measured. DMSO has no effect on Mtb growth in this assay and does not generate a zone of clearing on its own.

### C. Compound Synthesis

The compounds in Table 1 were purchased from ChemBridge (www.hit2lead.com).

TABLE 1
Compounds Obtained from ChemBridge with IUPAC Name and Catalog Number

| | | | ChemBridge |
|---|---|---|---|
| Example | | | Catalog |
| No. | Structure | IUPAC | Number |
| 0728 | 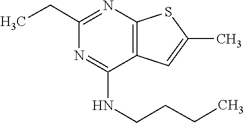 [SMILES: CCCCNc1nc(CC)nc2sc(C)cc12] | N-butyl-2-ethyl-6- methylthieno[2,3-d]pyrimidin- 4-amine | 9258457 |
| 0881 | 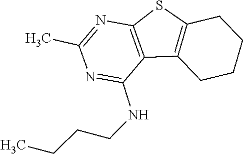 [SMILES: CCCCNc1nc(C)nc2sc3c(c12)CCCC3] | N-butyl-5-methyl-8-thia-4,6- diazatricyclo[7.4.0.0²,^{⁷}]trideca- 1(9),2,4,6-tetraen-3-amine | 7171934 |
| 0941 | 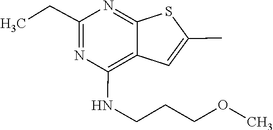 [SMILES: CCc1nc(NCCCOC)c2cc(C)sc2n1] | 2-ethyl-N-(3-methoxypropyl)-6- methylthieno[2,3-d]pyrimidin- 4-amine | 9245116 |
| 0950 | 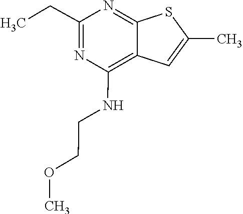 [SMILES: CCc1nc(NCCOC)c2cc(C)sc2n1] | 2-ethyl-N-(2-methoxyethyl)-6- methylthieno[2,3-d]pyrimidin- 4-amine | 9277737 |
| 0936 | 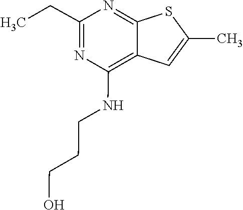 [SMILES: CCc1nc(NCCCO)c2cc(C)sc2n1] | 3-({2-ethyl-6-methylthieno[2,3- d]pyrimidin-4- yl}amino)propan-1-ol | 9220073 |
| 0951 | 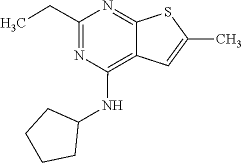 [SMILES: CCc1nc(NC2CCCC2)c2cc(C)sc2n1] | N-cyclopentyl-2-ethyl-6- methylthieno[2,3-d]pyrimidin- 4-amine | 9280331 |
| 0943 | 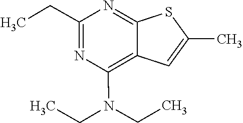 [SMILES: CCc1nc(N(CC)CC)c2cc(C)sc2n1] | N,N,2-triethyl-6- methylthieno[2,3-d]pyrimidin- 4-amine | 9253688 |
| 0942 | 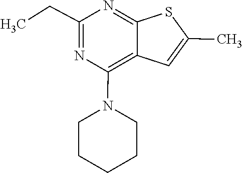 [SMILES: CCc1nc(N2CCCCC2)c2cc(C)sc2n1] | 1-{2-ethyl-6-methylthieno[2,3- d]pyrimidin-4-yl}piperidine | 9252275 |
| 0935 | 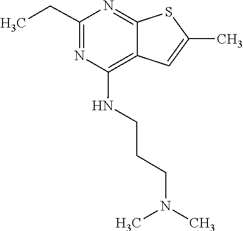 [SMILES: CCc1nc(NCCCN(C)C)c2cc(C)sc2n1] | [3-({2-ethyl-6- methylthieno[2,3-d]pyrimidin- 4-yl}amino)propyl]dimethylamine | 9219957 |
| 0851 | 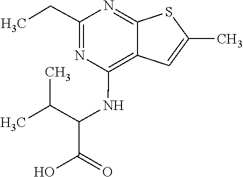 [SMILES: CCc1nc(NC(C(=O)O)C(C)C)c2cc(C)sc2n1] | 2-({2-ethyl-6-methylthieno[2,3- d]pyrimidin-4-yl}amino)-3- methylbutanoic acid | 9204833 |
| 0927 | 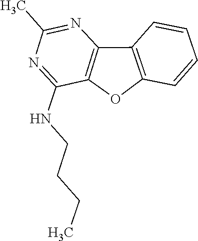 [SMILES: CCCCNc1nc(C)nc2c1oc1ccccc12] | N-butyl-4-methyl-8-oxa-3,5- diazatricyclo[7.4.0.0²,^{⁷}]trideca- 1(9),2(7),3,5,10,12-hexaen-6- amine | 9112687 |

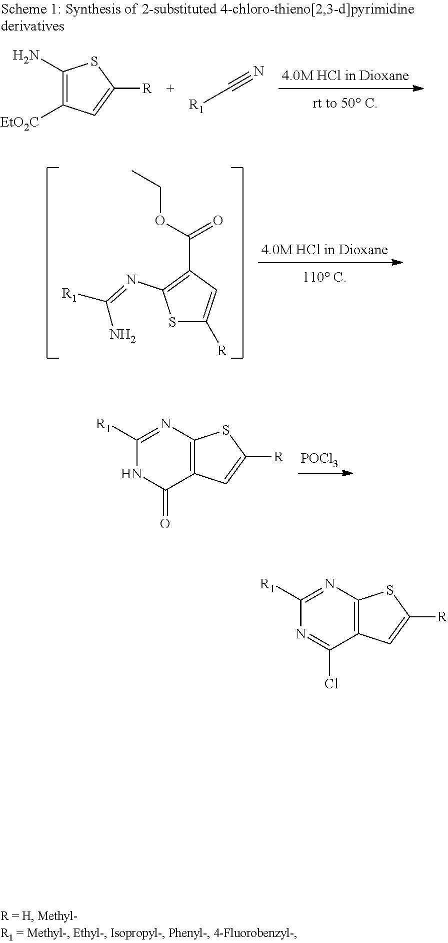

Scheme 1 shows a general method for the preparation of the thienopyrimidine intermediates from 2-aminothiophene-3-carboxyesters, appropriate alkyl nitriles and dry HCl in 1,4-dioxane. This procedure afforded the corresponding thienopyrimidinones which were converted to the corresponding 4-chloro-thienopyrimidines by the reaction of phosphorus oxychloride under refluxing conditions.

The 4-aminoalkyl derivatives were synthesized by the reactions of the appropriate 4-chloro-thienopyrimidines with alkyl amines in the presence of a tert-amine and the microwave heating methodology.

### Step 1. Preparation 2-ethyl-6-methyl-3H-thieno[2,3-d]pyrimidin-4-one

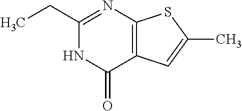

A dark red mixture of 2-amino-3-ethoxycarbonyl-5-methylthiophene (648.0 mg, 3.5 mmol) and propionitrile (0.5 mL, 7.0 mmol) was treated with 4.0 M HCl in 1,4-dioxane (3 mL, 12.0 mmol) at room temperature. The reaction mixture quickly turned to a thick yellow-orange paste. An additional 2 mL 4N HCl in 1,4-dioxane was added after 30 min and the reaction mixture was heated at 50° C. to give a red solution. LC-MS analysis of the reaction mixture after 1.5 h showed the uncyclized intermediate product and the intermediate's mass: m/z 241 (M+H), no traces of the starting materials were present. After heating at 50° C. for 2 h, the reaction mixture was heated at 110° C. to give a dark red solution. A thick yellow paste begin to form within 1 h. The reaction mixture was heated at 110° C. overnight. The solvent was evaporated in vacuo to afford a yellow-brown solid. The solid was dissolved in acetonitrile (20 mL) and cooled to room temperature to afford a crystalline precipitate. The solid was filtered, washed with acetonitrile (2×10 mL) and dried in vacuo to give a cream crystalline solid (648.0 mg, yield 95%). LC-MS analysis of the solid shows the desired product with a purity >98% and the desired product's mass: m/z 195 (M+H), and m/z 217 (M+Na); Calcd for C₉H₁₀N₂OS=194.25.

### Step 2. 4-Chloro-2-ethyl-6-methylthieno[2,3-d]pyrimidine

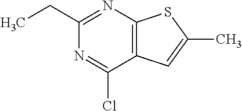

A suspension of 2-ethyl-6-methyl-3H-thieno[2,3-d]pyrimidin-4-one (648.0 mg, 3.34 mmol) in phosphorus oxychloride (4 mL, 42.91 mmol)) was heated at refluxing conditions. Within 1 h a light brown-orange solution was obtained. The solvent was evaporated in vacuo to afford a light orange-brown viscous liquid. The liquid was poured onto crushed ice-water to give a cream precipitate. The mixture was neutralized with a saturated NaHCO₃ solution to give a cream precipitate. The precipitate was extracted with ethyl acetate (2×25 mL), the aqueous and the organic layers were separated, the organic layer was washed with brine (1×25 mL) and dried over anhydrous Na₂SO₄, filtered and evaporated in vacuo to afford a very light brown liquid which solidified to a cream crystalline solid (620.0 mg, yield 88%). LC-MS analysis of the solid showed the desired product with a purity >98% and the desired product's mass: m/z 213 (^{35Cl}M+H), and m/z 215 (^{37Cl}M+H); Calcd for C₉H₉ClN₂S=212.70. ¹H NMR (400 MHz, DMSO-d₆): δ 1.30 (t, J=7.55 Hz, 3H, CH₃CH₂—), 2.63 (d, J=1.34 Hz, 3H, 6-CH₃—), 2.96 (q, J=7.54 Hz, 2H, CH₃CH₂—), 7.23 (d, J=1.34 Hz, 1H, H-5).

### 2-Ethyl-6-methyl-N-(3-phenylpropyl)thieno[2,3-d]pyrimidin-4-amine (1023)

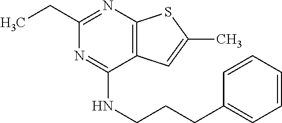 [SMILES: CCc1nc(NCCCc2ccccc2)c2cc(C)sc2n1]

A solution of 4-chloro-2-ethyl-6-methylthieno[2,3-d]pyrimidine (50 mg, 0.235 mmol), DIEA (50 μL, 0.292 mmol) and 3-phenyl-n-propylamine (100 μL, 0.705 mmol) in 1,4-dioxane was heated to 140° C. for 60 min in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug to neutralize TFA. Evaporation of the solvent in vacuo afforded the product as a white solid (32.1 mg, yield 44%). LC-MS analysis of the solid showed the desired product with a purity >98% and the desired product's mass: m/z 312 (M+H); Calcd for C₁₈H₂₁N₃S=311.44. ¹H NMR (400 MHz, DMSO-d₆): δ 7.64 (t, J=5.47 Hz, 1H), 7.12-7.35 (m, 6H), 3.44-3.54 (m, 2H), 2.67 (qd, J=3.79, 11.37 Hz, 4H), 2.50 (d, J=1.16 Hz, 3H), 1.86-1.99 (m, 2H), 1.22 (t, J=7.58 Hz, 3H).

### 2-Ethyl-6-methyl-N-(4,4,4-trifluorobutyl)thieno[2,3-d]pyrimidin-4-amine (1020)

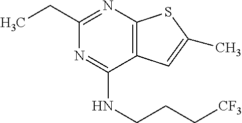 [SMILES: CCc1nc(NCCCC(F)(F)F)c2cc(C)sc2n1]

A solution of 4-chloro-2-ethyl-6-methyl-thieno[2,3-d]pyrimidine (50 mg, 0.235 mmol), DIEA (50 μL, 0.292 mmol) and 4,4,4-trifluorobutylamine (66 μL, 0.705 mmol) in 1,4-dioxane was heated to 140° C. for 60 min in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug. Evaporation of the solvent in vacuo afforded the product as a white solid (26.1 mg, yield 37%). LC-MS analysis of the solid showed the desired product with a purity >98% and the desired product's mass: m/z 304 (M+H); Calcd for C₁₃H₁₆F₃N₃S=303.35. ¹H NMR (400 MHz, DMSO-d₆): δ 7.68 (t, J=5.53 Hz, 1H), 7.18 (d, J=1.34 Hz, 1H), 3.53 (q, J=6.66 Hz, 2H), 2.67 (q, J=7.56 Hz, 2H), 2.49 (d, J=1.00 Hz, 3H), 2.24-2.44 (m, 2H), 1.76-1.90 (m, 2H), 1.22 (t, J=7.55 Hz, 3H); ¹⁹F NMR (376 MHz, DMSO-d₆): δ −64.68.

### 2-Ethyl-6-methyl-N-phenethylthieno[2,3-d]pyrimidin-4-amine (1306)

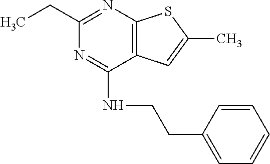 [SMILES: CCc1nc(NCCc2ccccc2)c2cc(C)sc2n1]

A solution of 4-chloro-2-ethyl-6-methyl-thieno[2,3-d]pyrimidine (67.0 mg, 0.32 mmol), DIEA (110 μL, 0.64 mmol) and 2-phenylethylamine (119 μL, 0.95 mmol) in 1,4-dioxane (2 mL) was heated at 140° C. in a microwave reactor for 3 h to give a colorless solution and the solvent was evaporated in vacuo to give a pale viscous liquid. The crude residue was purified by reverse-phase preparative HPLC to afford a colorless crystalline solid (132.0 mg). The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (90.0 mg, yield 96%). LC-MS analysis of the freebase product showed the desired product with a purity >98% and the desired product's mass: m/z 298 (M+H); Calcd for C₁₇H₁₉N₃S=207.42. ¹H NMR (400 MHz, CDCl₃): δ 1.39 (t, J=7.58 Hz, 3H, CH₃—CH₂—), 2.53 (s, 3H, 6-CH₃—), 2.87 (q, J=7.58 Hz, 2H, CH₃—CH₂—), 2.99 (t, J=6.97 Hz, 2H, —CH₂—CH₂-Ph), 3.89 (q, J=6.60 Hz, 2H, —NH—CH₂—CH₂-Ph), 4.95 (brs/appt, 1H, —NH—), 6.60 (s, 1H, H-5), 7.23-7.29 (m, 3H, Ph-H), 7.32-7.39 (m, 2H, Ph-H).

### 2-Ethyl-6-methyl-N-(3-methylbutyl)thieno[2,3-d]pyrimidin-4-amine (1022)

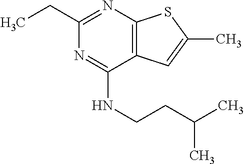 [SMILES: CCc1nc(NCCC(C)C)c2cc(C)sc2n1]

A solution of 4-chloro-2-ethyl-6-methyl-thieno[2,3-d]pyrimidine (50 mg, 0.235 mmol), DIEA (50 μL, 0.292 mmol) and isopentylamine (82 μL, 0.705 mmol) in 1,4-dioxane was heated to 140° C. for 60 min in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug to neutralize TFA. Evaporation of the solvent in vacuo afforded the product as a white solid (29.0 mg, yield 47%). LC-MS analysis of the solid showed the desired product with a purity >98% and the desired product's mass: m/z 264 (M+H); Calcd for C₁₄H₂₁N₃S=263.40. ¹H NMR (400 MHz, DMSO-d₆): δ 7.54 (t, J=5.44 Hz, 1H), 7.19 (d, J=1.22 Hz, 1H), 3.42-3.55 (m, 2H), 2.66 (q, J=7.58 Hz, 2H), 2.48 (s, 3H), 1.63 (td, J=6.65, 13.36 Hz, 1H), 1.48 (q, J=6.85 Hz, 2H), 1.22 (t, J=7.58 Hz, 3H), 0.92 (d, J=6.54 Hz, 6H).

### N-[2-(2,4-Dichlorophenyl)ethyl]-2-ethyl-6-methylthieno[2,3-d]pyrimidin-4-amine (1307)

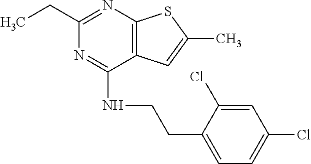 [SMILES: CCc1nc(NCCc2ccc(Cl)cc2Cl)c2cc(C)sc2n1]

A solution of 4-chloro-2-ethyl-6-methyl-thieno[2,3-d]pyrimidine (65.2 mg, 0.31 mmol), DIEA (107 μL, 0.63 mmol) and 2,4-dichlorophenethylamine (138 μL, 0.92 mmol) in 1,4-dioxane (2 mL) was heated at 140° C. in a microwave reactor for 3 h to give a pale yellow solution and the solvent was evaporated in vacuo to give a yellow-orange viscous liquid. The crude residue was purified by reverse-phase preparative HPLC to afford a colorless crystalline solid. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (109.3 mg; yield 97%). LC-MS analysis of the freebase product showed the desired product with a purity >98% and desired product's mass: m/z 366 (^{35Cl}M+H) and m/z 368 (^{37Cl}M+H); Calcd for C₁₇H₁₇Cl₂N₃S=366.30. ¹H NMR (400 MHz, CDCl₃): δ 1.37 (t, J=7.58 Hz, 3H, CH₃—CH₂—), 2.54 (d, J=0.98 Hz, 3H, 6-CH₃—), 2.86 (q, J=7.58 Hz, 2H, CH₃—CH₂—), 3.12 (t, J=6.97 Hz, 2H, —CH₂—CH₂-Ph), 3.87 (q, J=6.85 Hz, 2H, —NH—CH₂—CH₂-Ph), 5.00 (t, J=5.62 Hz, 1H, —NH—), 6.64 (d, J=1.22 Hz, 1H, H-5), 7.15-7.21 (m, 2H, Ph-H), 7.41 (d, J=0.98 Hz, 1H, Ph-H).

### N-Cyclopropyl-2-ethyl-6-methylthieno[2,3-d]pyrimidin-4-amine (0946)

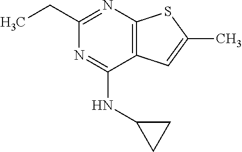 [SMILES: CCc1nc(NC2CC2)c2cc(C)sc2n1]

A solution of 4-chloro-2-ethyl-6-methyl-thieno[2,3-d]pyrimidine (50 mg, 0.235 mmol), DIEA (50 μL, 0.292 mmol) and cyclopropylamine (49 μL, 0.705 mmol) in 1,4-dioxane was heated to 140° C. for 2 h in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug to neutralize. Evaporation of the solvent in vacuo afforded a tan solid (22.9 mg, yield 42%). LC-MS analysis of the solid showed the desired product with a purity >98% and the desired product's mass: m/z 234 (M+H); Calcd for C₁₂H₁₅N₃S=233.33. ¹H NMR (400 MHz, DMSO-d₆): δ 7.66 (d, J=3.36 Hz, 1H), 7.20 (s, 1H), 2.97 (dt, J=3.70, 7.14 Hz, 1H), 2.69 (q, J=7.58 Hz, 2H), 2.48 (d, J=1.22 Hz, 3H), 1.24 (t, J=7.58 Hz, 3H), 0.71-0.80 (m, 2H), 0.52-0.59 (m, 2H).

### N-(4-Chlorobenzyl)-2-ethyl-6-methylthieno[2,3-d]pyrimidin-4-amine (1302)

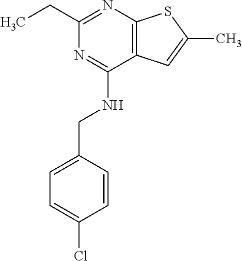 [SMILES: CCc1nc(NCc2ccc(Cl)cc2)c2cc(C)sc2n1]

A solution of 4-chloro-2-ethyl-6-methyl-thieno[2,3-d]pyrimidine 71.0 mg, 0.33 mmol), DIEA (120 μL, 0.70 mmol) and 4-chlorobenzylamine (125 μL, 1.03 mmol) in 1,4-dioxane (2 mL) was heated at 140° C. in a microwave reactor (normal power) for 3 h to give a pale yellow solution and the solvent was evaporated in vacuo to give a cream crystalline solid. The crude residue was purified by reverse-phase preparative HPLC to afford a colorless crystalline solid suspended in water. The solid was filtered, washed with water (2×10 mL) and dried in vacuo to afford a colorless crystalline solid. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (100.1 mg, yield 94%). LC-MS analysis of the freebase product showed the desired product with a purity >98% and the desired product's mass: m/z 318 (^{35Cl}M+H) and m/z 320 (^{37Cl}M+H); Calcd for C₁₆H₁₆ClN₃S=317.84 ¹H NMR (400 MHz, CDCl₃): δ 1.35 (t, J=7.46 Hz, 3H, CH₃—CH₂—), 2.54 (s, 3H, 6-CH₃—), 2.86 (q, J=7.58 Hz, 2H, CH₃—CH₂—), 4.81 (d, J=5.87 Hz, 2H, —NH—CH₂—), 6.71 (s, 1H, —NH—), 7.28-7.38 (dd/m, 4H, Ph-H-2, H-3, H-5, H-6).

### N-Butyl-2-ethyl-N,6-dimethylthieno[2,3-d]pyrimidin-4-amine (1021)

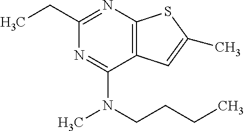 [SMILES: CCCCN(C)c1nc(CC)nc2sc(C)cc12]

A solution of 4-chloro-2-ethyl-6-methyl-thieno[2,3-d]pyrimidine (50 mg, 0.235 mmol), DIEA (50 μL, 0.292 mmol) and N-methyl-n-butylamine (84 μL, 0.705 mmol) in 1,4-dioxane was heated to 140° C. for 60 min in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug to neutralize. Evaporation of the solvent in vacuo afforded as an oil which was dissolved in acetonitrile/water and lyophilized to give a white solid (24.8 mg, yield 40%). LC-MS analysis of the solid showed the desired product with a purity >98% and the desired product's mass: m/z 264 (M+H); Calcd for C₁₄H₂₁N₃S=263.40. ¹H NMR (400 MHz, DMSO-d₆): δ 7.24 (d, J=1.28 Hz, 1H), 3.66-3.77 (m, 2H), 3.32 (s, 3H), 2.66 (q, J=7.58 Hz, 2H), 2.49 (d, J=1.16 Hz, 3H), 1.55-1.65 (m, 2H), 1.27-1.39 (m, 2H), 1.23 (t, J=7.58 Hz, 3H), 0.92 (t, J=7.37 Hz, 3H).

### 2-Ethyl-6-methyl-thieno[2,3-d]pyrimidin-4-amine (1060)

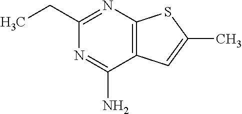

A suspension of 4-chloro-6-methyl-2-phenyl-thieno[2,3-d]pyrimidine (59.0 mg, 0.28 mmol) in 1,4-dioxane (1 mL) and ammonium hydroxide solution (28%) (2 mL, 16.5 mmol) was heated at 80° C. in a microwave reactor for 1 h to give a colorless solution. The solvent was evaporated in vacuo to afford a colorless crystalline residue. The crude residue was purified by reverse-phase preparative HPLC to afford a colorless crystalline solid. The purified product was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline powder (48.5 mg; yield 91%). LC-MS analysis of the solid showed the desired product with a purity >98% and the desired product's mass: m/z 194 (M+H); Calcd for C₉H_{II}N₃S=193.27. ¹H NMR (400 MHz, DMSO-d₆): δ1.21 (t, J=7.58 Hz, 3H, CH₃—CH₂—), 2.48 (d, J=1.22 Hz, 3H, 6-CH₃—), 2.64 (q, J=7.58 Hz, 2H, CH₃—CH₂—), 7.15 (d, J=1.22 Hz, 1H, H-5), 7.20 (s, 2H, 4-NH₂).

### 2-Ethyl-6-methyl-N-phenylthieno[2,3-d]pyrimidin-4-amine (1304)

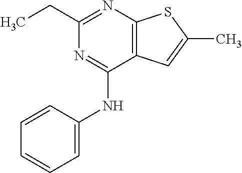 [SMILES: CCc1nc(Nc2ccccc2)c2cc(C)sc2n1]

A solution of 4-chloro-2-ethyl-6-methyl-thieno[2,3-d]pyrimidine (71.6 mg, 0.34 mmol), DIEA (120 μL, 0.70 mmol) and aniline (95 μL, 1.04 mmol) in 1,4-dioxane (2 mL) was heated at 200° C. in a microwave reactor for 6 h to give a dark yellow solution. The solvent was evaporated in vacuo to give a pale yellow viscous liquid solidified slowly to a dirty cream crystalline solid (176.6 mg). The crude residue was purified by reverse-phase preparative HPLC to afford a colorless crystalline precipitate in water. The solid was filtered, washed with water (3×10 mL) and dried in vacuo to afford a colorless solid. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a cream crystalline solid (88.9 mg; yield 98%). LC-MS analysis of the freebase product showed the desired product with a purity >98% and the desired product's mass: m/z 270 (M+H); Calcd for C₁₅H₁₅N₃S=269.37. ¹H NMR (400 MHz, CDCl₃): δ 1.41 (t, J=7.58 Hz, 3H, CH₃—CH₂—), 2.56 (s, 3H, 6-CH₃—), 2.94 (q, J=7.58 Hz, 2H, CH₃—CH₂—), 6.73 (s, 1H, H-5), 6.80 (brs, 1H, —NH—), 7.14 (t, J=7.45 Hz, 1H, Ph-H4), 7.39 (t, J=7.70 Hz, 2H, Ph-H3, H5), 7.71 (d, J=8.31 Hz, 2H, Ph-H-2, H-6).

### 2-Ethyl-6-methyl-N-(2,2,2-trifluoroethyl)thieno[2,3-d]pyrimidin-4-amine (1072)

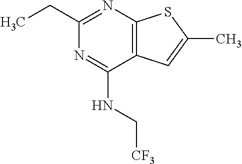 [SMILES: CCc1nc(NCC(F)(F)F)c2cc(C)sc2n1]

A solution of 4-chloro-2-ethyl-6-methyl-thieno[2,3-d]pyrimidine (65.0 mg, 0.31 mmol), DIEA (80 μL, 0.47 mmol) and 2,2,2-trifluoroethanamine (75 μL, 0.94 mmol) in 1,4-dioxane (1.5 mL) was heated at 200° C. in a microwave reactor for several hours (>24 h) for a decent conversion (>92%) to give a pale yellow solution. The solvent was evaporated in vacuo to afford a light tan crystalline solid. The crude residue was purified by reverse-phase preparative HPLC to afford a colorless to cream crystalline residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless to cream crystalline solid (44.0 mg, yield 52.3%). LC-MS analysis of the freebase product showed the desired product with a purity >98% and the desired product's mass: m/z 276 (M+H); Calcd for C₁₁H₁₂F₃N₃S=275.29. ¹H NMR (400 MHz, DMSO-d₆): δ 1.23 (t, J=7.58 Hz, 3H, CH₃—CH₂—), 2.52 (d, J=0.98 Hz, 3H, 6-CH₃—), 2.72 (q, J=7.58 Hz, 2H, CH₃—CH₂—), 4.37 (qd, J=9.70 and 6.60 Hz, 2H, —CH₂—CF₃), 7.28 (d, J=1.22 Hz, 1H, H-5), 8.20 (t, J=6.36 Hz, 1H, —NH—CH₂—CF₃); ¹⁹F NMR (376 MHz, DMSO-d₆): δ−70.35 (t, J=10.0 Hz, 3F, CF₃—).

### N-Benzyl-2-ethyl-6-methylthieno[2,3-d]pyrimidin-4-amine (1303)

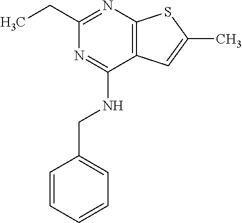 [SMILES: CCc1nc(NCc2ccccc2)c2cc(C)sc2n1]

A solution of 4-chloro-2-ethyl-6-methyl-thieno[2,3-d]pyrimidine (67.0 mg, 0.315 mmol), DIEA (110 μL, 0.643 mmol) and benzylamine (105 μL, 0.96 mmol) in 1,4-dioxane (2 mL) was heated at 140° C. in a microwave reactor for 3 h to give a pale yellow solution. The solvent was evaporated in vacuo to give a cream crystalline solid. The crude residue was purified by reverse-phase preparative HPLC to afford a colorless viscous liquid containing a colorless crystalline solid (130.3 mg). The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless crystalline solid (90.0 mg, yield 100%). LC-MS analysis of the freebase product showed the desired product with a purity >98% and the desired product's mass: m/z 284 (M+H); Calcd for C₁₆H₁₇N₃S=283.39. ¹H NMR (400 MHz, CDCl₃): δ 1.37 (t, J=7.58 Hz, 3H, CH₃—CH₂—), 2.53 (s, 3H, 6-CH₃—), 2.87 (q, J=7.58 Hz, 2H, CH₃—CH₂—), 4.84 (d, J=5.62 Hz, 2H, —CH₂—NH—), 5.17 (brs/appt, 1H, —NH—), 6.71 (s, 1H, H-5), 7.28-7.37 (m, 3H, Ph-), 7.38-7.42 (m, 2H, Ph-).

### N-Cyclobutyl-2-ethyl-6-methylthieno[2,3-d]pyrimidin-4-amine (1019)

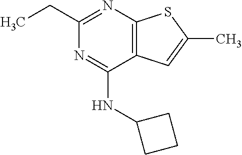 [SMILES: CCc1nc(NC2CCC2)c2cc(C)sc2n1]

A solution of 4-chloro-2-ethyl-6-methyl-thieno[2,3-d]pyrimidine (50 mg, 0.235 mmol), DIEA (50 μL, 0.292 mmol) and cyclobutylamine (60 μL, 0.705 mmol) in 1,4-dioxane was heated to 140° C. for 60 min in a microwave reactor. The reaction mixture was partitioned between water and DCM. The DCM layer was separated and concentrated and the crude product was purified by reverse phase HPLC to give the desired product. The residue was dissolved in acetonitrile containing a couple drops of methanol and eluted through a SiliaPrep Carbonate 6 mL-1 g plug to neutralize. Evaporation of the solvent in vacuo afforded a white solid (27.3 mg, yield 47%) LC-MS analysis of the solid showed the desired product with a purity >98% and the desired product's mass: m/z 248 (M+H); Calcd for C₁₃H₁₇N₃S=247.36. ¹H NMR (400 MHz, DMSO-d₆): δ 7.73 (d, J=7.21 Hz, 1H), 7.24 (d, J=1.28 Hz, 1H), 4.56-4.73 (m, 1H), 2.66 (q, J=7.56 Hz, 2H), 2.49 (d, J=1.10 Hz, 3H), 2.21-2.36 (m, 2H), 1.95-2.12 (m, 2H), 1.64-1.78 (m, 2H), 1.22 (t, J=7.58 Hz, 3H).

### N-Benzyl-2,5,6-trimethylthieno[2,3-d]pyrimidin-4-amine (0795)

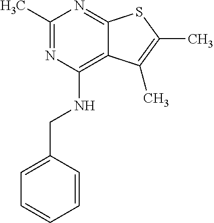

A solution of 4-chloro-2,5,6-trimethyl-thieno[2,3-d]pyrimidine (50 mg, 0.24 mmol), benzylamine (26 μL, 0.24 mmol), DIEA (46 μL, 0.26 mmol) in dioxane (500 μL) was heated to 120° C. in a microwave reactor until a >95% conversion was achieved (1 h). The reaction was concentrated and mixture was partitioned between water and ethyl acetate. The organic layer was removed and concentrated to give a yellow solid. The crude product was purified by reverse phase HPLC to give the final product as an off white powder (66.2 mg, yield 77%, TFA salt). LC-MS analysis of the solid showed the desired product with a purity >98% and the desired product's mass: m/z 284 (M+H); Calcd for C₁₆H₁₇N₃S=283.39. ¹H NMR (400 MHz, DMSO-d₆): δ 7.84 (br. s., 1H), 7.36-7.42 (m, 2H), 7.29-7.35 (m, 2H), 7.21-7.26 (m, 1H), 4.79 (d, J=5.93 Hz, 2H), 2.48 (s, 3H), 2.44 (s, 3H), 2.40 (s, 3H).

### 2-Ethyl-6-methyl-N-(2-pyridylmethyl)thieno[2,3-d]pyrimidin-4-amine (1308)

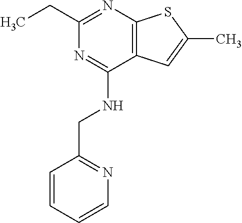 [SMILES: CCc1nc(NCc2ccccn2)c2cc(C)sc2n1]

A solution of 4-chloro-2-ethyl-6-methyl-thieno[2,3-d]pyrimidine (67.7 mg, 0.32 mmol), DIEA (115 μL, 0.67 mmol) and 2-pyridylmethanamine (95 μL, 0.97 mmol) in 1,4-dioxane (2 mL) was heated at 140° C. in a microwave reactor for 3 h. The solvent was evaporated in vacuo to give a yellow-orange crystalline/gummy residue. The crude residue was purified by reverse-phase preparative HPLC to afford a pale pink viscous liquid. The purified product was dissolved in acetonitrile containing a trace of methanol and the solution was passed through a SiliaPrep Carbonate (Si—CO₃) 6 mL-1 g cartridge. The filtrate was evaporated in vacuo to afford a beige foamy solid (124.8 mg). LC-MS analysis of the freebase product showed the desired product with a purity >98% and the desired product's mass: m/z 285 (M+H); Calcd for C₁₅H₁₆N₄S=284.38. ¹H NMR (400 MHz, CDCl₃): δ 1.36 (t, J=7.58 Hz, 3H, CH₃—CH₂—), 2.55 (d, J=0.98 Hz, 3H, 6-CH₃—), 2.92 (q, J=7.50 Hz, 2H, CH₃—CH₂—), 4.99 (d, J=5.38 Hz, 2H, —CH₂—NH—), 7.04 (s, 1H, H-5) 7.41 (t, J=6.11 Hz, 1H, Py-H-5) 7.65 (d, J=8.07 Hz, 1H, Py-H-4), 7.85-7.97 (m/appt, 1H, Py-H-3), 8.61 (d, J=5.13 Hz, 1H, Py-H-6); peak due to —NH— is hidden under baseline.

### 2,5,6-Trimethyl-N-(2-phenylethyl)thieno[2,3-d]pyrimidin-4-amine (0798)

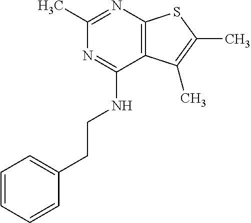 [SMILES: Cc1nc(NCCc2ccccc2)c2c(C)c(C)sc2n1]

A solution of 4-chloro-2,5,6-trimethyl-thieno[2,3-d]pyrimidine (50 mg, 0.235 mmol), 2-phenylethanamine (89.0 μL, 0.705 mmol), DIEA (50 μL, 0.292 mmol) in 1,4-dioxane (500 μL) was heated to 140° C. for 60 min in microwave reactor. The reaction mixture partitioned between water and DCM. The DCM layer was separated and evaporated in vacuo. The crude product was purified by reverse phase HPLC to give the final product as a white solid (16 mg, yield 16%, TFA salt). LC-MS of the solid showed the desired product with a purity >98% and the desired product's mass: 298 (M+H); Calcd for C₁₇H₁₉N₃S=297.42. ¹H NMR (400 MHz, DMSO-d₆): δ 7.17-7.35 (m, 5H), 3.72-3.82 (m, 2H), 2.94 (t, J=7.37 Hz, 2H), 2.49 (s, 3H), 2.38 (s, 3H), 2.36 (s, 3H)

### N-tert-Butyl-2-ethyl-6-methyl-thieno[2,3-d]pyrimidin-4-amine (1069)

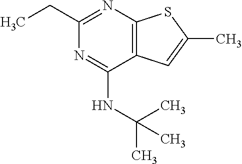 [SMILES: CCc1nc(NC(C)(C)C)c2cc(C)sc2n1]

A solution of 4-chloro-2-ethyl-6-methyl-thieno[2,3-d]pyrimidine (60.5 mg, 0.28 mmol), DIEA (100 μL, 0.58 mmol) and tert-butylamine (90 μL, 0.85 mmol) in 1,4-dioxane (1 mL) was heated at from 140 to 200° C. in a microwave reactor for several hours (>18 h) to give a yellow-orange solution. The solvent was evaporated in vacuo to give a light tan crystalline solid and the crude residue was purified by reverse-phase preparative HPLC to afford a colorless to cream crystalline residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless powder (37.3 mg, yield 53%). LC-MS analysis of the freebase product shows the desired product with a purity >98% and the desired product's mass: m/z 250 (M+H); Calcd for C₁₃H₁₉N₃S=249.39. ¹H NMR (400 MHz, DMSO-d₆): δ 1.24 (t, J=7.58 Hz, 3H, CH₃—CH₂—), 1.50 (s, 9H, (CH₃)₃C—), 2.48 (d, J=1.22 Hz, 3H, 6-CH₃—), 2.68 (q, J=7.58 Hz, 2H, CH₃—CH₂—), 6.82 (s, 1H, —NH—), 7.37 (d, J=1.22 Hz, 1H, H-5).

### 2,5,6-Trimethyl-N-phenylthieno[2,3-d]pyrimidin-4-amine (0797)

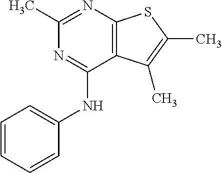 [SMILES: Cc1nc(Nc2ccccc2)c2c(C)c(C)sc2n1]

A solution of 4-chloro-2,5,6-trimethyl-thieno[2,3-d]pyrimidine (50 mg, 0.235 mmol), aniline (65 μL, 0.705 mmol), DIEA (50 μL, 0.292 mmol) in 1,4-dioxane (500 μL) was heated to 180° C. for 2 h in microwave reactor. The reaction mixture partitioned between water and DCM. The DCM layer was separated and evaporated in vacuo. The crude product was purified by reverse phase HPLC to give the final product as tan needles (23.6 mg, yield 24%, TFA salt). LC-MS of the solid showed the desired product with a purity >98% and the desired product's mass: 270 (M+H); Calcd for C₁₅H₁₅N₃S=269.36. NMR (400 MHz, DMSO-d₆): δ 8.38 (s, 1H), 7.67-7.72 (m, 2H), 7.33-7.40 (m, 2H), 7.08-7.13 (m, 1H), 2.57 (d, J=0.73 Hz, 3H), 2.46 (s, 3H), 2.43 (d, J=0.55 Hz, 3H).

### N-butyl-2,5,6-trimethylthieno[2,3-d]pyrimidin-4-amine trifluoroacetate (0796)

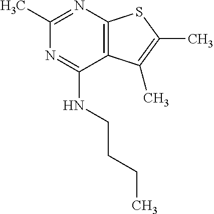

A solution of 4-chloro-2,5,6-trimethyl-thieno[2,3-d]pyrimidine (50 mg, 0.235 mmol), butan-1-amine (69.7 μL, 0.705 mmol), DIEA (50 μL, 0.292 mmol) in 1,4-dioxane (500 μL) was heated to 140° C. for 60 min in microwave reactor. The reaction mixture partitioned between water and DCM. The DCM layer was separated and evaporated in vacuo. The crude product was purified by reverse phase HPLC to give the final product as an orange waxy solid (72 mg, yield 81%, TFA salt). LC-MS of the solid showed the desired product with a purity >94% and the desired product's mass: 250 (M+H); Calcd for C₁₃H₁₉N₃S=249.39. ¹H NMR (400 MHz, DMSO-d₆): δ 7.48 (br. s., 1H), 3.54-3.61 (m, 2H), 2.50 (s, 3H), 2.44 (s, 3H), 2.40 (s, 3H), 1.57-1.66 (m, 2H), 1.30-1.41 (m, 2H), 0.92 (t, J=7.37 Hz, 3H).

### Step 1. Preparation 2-ethyl-3H-thieno[2,3-d]pyrimidin-4-one

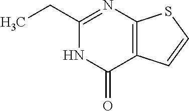

A dark red mixture of methyl 2-aminothiophene-3-carboxylate (1.04 g, 6.64 mmol) and propionitrile (0.70 ml, 9.82 mmol) was treated with 4.0 M HCl in 1,4-dioxane (3 mL, 12.00) at room temperature. The reaction mixture quickly turned to a thick dark brown black paste. An additional 3 mL 4N HCl in 1,4-dioxane was added after 30 min and the reaction mixture was heated at 50° C. to give a purple black suspension. LC-MS analysis of the reaction mixture after overnight stirring showed the uncyclized intermediate product and the intermediate's mass: m/z 213 (M+H) and m/z 181 (M+H—CH₃OH). Another 3 mL of 4.0 N HCl in 1,4-dioxane was added and the mixture was heated at 110° C. A solid begin to form within 1 h. The reaction mixture was heated at 110° C. for another 5 h to give a dark green suspension. The solvent was evaporated in vacuo to give a dark green residue. The residue was triturated with acetonitrile (25 mL) to give an olive green precipitate. The solid/precipitate was filtered, washed with acetonitrile (2×10 mL) and dried in vacuo to give a dirty dark green solid. The solid was triturated with acetonitrile (25 mL) to give an olive green precipitate. The solid w was filtered, washed with acetonitrile (1×10 mL) and dried in vacuo to give an olive green solid (734.0 mg, yield 62%). LC-MS analysis of the solid showed the desired product with a purity >95% and the desired product's mass: m/z 181 (M+H), and m/z 203 (M+Na); Calcd for C₈H₈N₂S: 180.23. ¹H NMR (400 MHz, DMSO-d₆): δ 1.22 (t, J=7.46 Hz, 3H, CH₃—CH₂—), 2.64 (q, J=7.58 Hz, 2H, CH₃—CH₂—), 7.33 (d, J=5.87 Hz, 1H, H-5), 7.47 (d, J=5.62 Hz, 1H, H-6), 12.35 (brs, 1H, —NH—);

### Step 2. 4-Chloro-2-ethyl-thieno[2,3-d]pyrimidine

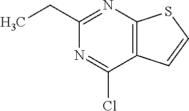

A suspension of 2-ethyl-3H-thieno[2,3-d]pyrimidin-4-one (390.4 mg, 2.17 mmol) in phosphorus oxychloride (5 mL, 54.0 mmol) was heated at refluxing conditions. Within 15 min a very pale yellow solution was obtained. The reaction mixture was cooled and poured onto crushed ice-water to give a colorless precipitate. The mixture was neutralized with a saturated NaHCO₃ solution to give a colorless precipitate. The precipitate was extracted with ethyl acetate (2×25 mL), the aqueous and the organic layers were separated. The organic layer was washed with brine (1×10 mL) and dried over anhydrous Na₂SO₄, filtered and evaporated in vacuo to afford a colorless to cream crystalline solid (414.0 mg, yield 96%). LC-MS analysis of the residue showed the desired product with a purity >98% and the desired product's mass: m/z 199 (^{35Cl}M+H), and m/z 201 (^{37Cl}M+H); Calcd for C₈H₇ClN₂S=198.67. ¹H NMR (400 MHz, DMSO-d₆): δ 1.32 (t, J=7.46 Hz, 3H, CH₃CH₂—), 2.99 (q, J=7.58 Hz, 2H, CH₃CH₂—), 7.52 (d, J=6.11 Hz, 1H), 8.02 (d, J=6.11 Hz, 1H).

### N-Cyclopropyl-2-ethyl-thieno[2,3-d]pyrimidin-4-amine (1074)

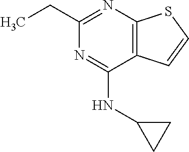

A solution of 4-chloro-2-ethyl-thieno[2,3-d]pyrimidine (67.0 mg, 0.34 mmol), DIEA (80.0 μL, 0.47 mmol) and cyclopropylamine (75 μL, 1.07 mmol) in 1,4-dioxane (1.5 mL) was heated at 140° C. in a microwave reactor for 3 h to give a yellow-orange solution. The solvent was evaporated in vacuo to afford an orange viscous residue. The crude residue was purified by reverse-phase preparative HPLC to afford a colorless to cream gummy residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless to cream crystalline solid (55.0 mg, yield 74%). LC-MS analysis of the residue showed the desired product with a purity >98% and the desired product's mass: m/z 220 (M+H); Calcd for C₁₁H₁₃N₃S=219.31. ¹H NMR (400 MHz, DMSO-d₆): δ 0.52-0.63 (m, 2H, —CH₂—), 0.73-0.84 (m, 2H, —CH₂—), 1.26 (t, J=7.58 Hz, 3H, CH₃—CH₂—), 2.73 (q, J=7.58 Hz, 2H, CH₃—CH₂—), 3.01 (td, J=7.21 and 3.67 Hz, 1H, —CH— (cypyl)), 7.42 (d, J=5.87 Hz, 1H, —CH—), 7.54 (d, J=5.62 Hz, 1H, —CH—), 7.90 (d, J=1.96 HZ, 1H, —NH—).

### N-butyl-2-ethyl-thieno[2,3-d]pyrimidin-4-amine (1176)

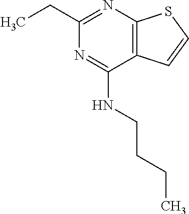 [SMILES: CCCCNc1nc(CC)nc2sccc12]

A solution of 4-chloro-2-ethyl-thieno[2,3-d]pyrimidine (59.6 mg, 0.30 mmol), DIEA (110 μL, 0.64 mmol) and butylamine (50 μL, 0.51 mmol) in 1,4-dioxane (1.5 mL) was heated at 140° C. in a microwave reactor for 3 h to give a colorless crystalline suspension and the solvent was evaporated in vacuo to give a pale yellow viscous liquid. The above liquid was partitioned between water (25 mL) and dichloromethane (25 mL). The DCM layer was removed and evaporated in vacuo to afford a very pale yellow film of the crude product. The crude residue was purified by reverse-phase preparative HPLC to afford a colorless glassy residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless powder (77.6 mg, yield 100%). LC-MS analysis of the freebase product showed the desired product with a purity >98% and the desired product's mass: m/z 236 (M+H); Calcd for C₁₂H₁₇N₃S=235.35. ¹H NMR (400 MHz, DMSO-d₆): δ 0.92 (t, J=7.58 Hz, 3H, CH₃—CH₂—CH₂—), 1.24 (t, J=7.58 Hz, 3H, CH₃—CH₂—), 1.36 (dq, J=14.86 Hz and 7.36 Hz, 2H, (—CH₂—CH₃), 1.59 (quint, J=7.21 Hz, 2H, —CH₂—CH₂—), 2.69 (q, J=7.58 Hz, 2H, CH₃—CH₂—), 3.45-3.54 (m, 2H, —CH₂—NH—), 7.42 (d, J=5.87 Hz, 1H, H-5), 7.54 (d, J=5.87 Hz, 1H, H-6), 7.81 (t, J=5.38 Hz, 1H, —NH—).

### 2-Ethyl-N-(3-phenylpropyl)thieno[2,3-d]pyrimidin-4-amine (1305)

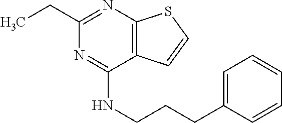 [SMILES: CCc1nc(NCCCc2ccccc2)c2ccsc2n1]

A solution of 4-chloro-2-ethyl-thieno[2,3-d]pyrimidine (60.0 mg, 0.30 mmol), DIEA (105 μL, 0.62 mmol) and 3-phenylpropan-1-amine in (130 μL, 0.92 mmoL) 1,4-dioxane (2.00 mL) was heated at 140° C. in a microwave reactor for 3 h to give a yellow solution and the solvent was evaporated in vacuo to afford a yellow viscous residue. The crude residue was purified by reverse-phase preparative HPLC to afford a colorless viscous liquid. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (79.7 mg, yield 89%). LC-MS analysis of the freebase product showed the desired product with a purity >98% and the desired product's mass: m/z 298 (M+H); Calcd for C₁₇H₁₉N₃S=297.42. ¹H NMR (400 MHz, CDCl₃): δ 1.37 (t, J=7.58 Hz, 3H, CH₃—CH₂—), 2.06 (quip, J=7.21 Hz, 2H, —CH₂—CH₂—CH₂—), 2.78 (t, J=7.46 Hz, 2H, —CH₂—CH₂—CH₂-Ph), 2.87 (q, J=7.42 Hz, 2H, CH₃—CH₃—), 3.71 (q, J=6.52 Hz, 2H, —NH—CH₂—CH₂—CH₂-Ph), 5.02 (brs, 1H, —NH—), 6.94 (d, J=5.87 Hz, 1H, H-6), 7.15 (d, J=5.87 Hz, 1H, H-5), 7.20-7.26 (m, 3H, Ph-H), 7.28-7.35 (m, 2H, Ph-H).

### 2-Ethyl-N-(4,4,4-trifluorobutyl)thieno[2,3-d]pyrimidin-4-amine (1366)

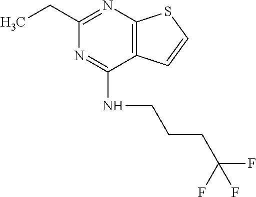 [SMILES: CCc1nc(NCCCC(F)(F)F)c2ccsc2n1]

A solution of 4-chloro-2-ethylthieno[2,3-d]pyrimidine (68.0 mg, 0.34 mmol), DIEA (100 μL, 0.58 mmol) and 4,4,4-trifluorobutan-1-amine (120 μL, 1.05 mmol) in 1,4-dioxane (2.0 mL) was heated at 160° C. in a microwave reactor for 3 h and the solvent was evaporated in vacuo to afford a pale yellow crystalline solid. The crude residue was purified by reverse-phase preparative HPLC on a purified to afford a colorless viscous liquid. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (108.0 mg, yield 100%). LC-MS analysis of the freebase residue showed the desired product with a purity >98% and the desired product's mass: m/z 290 (M+H); Calcd for C₁₂H₁₄F₃N₃S=289.32. ¹H NMR (400 MHz, DMSO-d₆): δ 1.35 (t, J=7.58 Hz, 3H, CH₃—CH₂—), 1.96 (quin, J=7.40 Hz, 2H, —CH₂—CH₂—CH₂—), 2.36-2.52 (m, 2H, —CH₂—CH₂—CH₂—), 2.81 (q, J=7.58 Hz, 2H, CH₃—CH₂—), 3.66 (q, J=6.60 Hz, 2H, —CH₂—CF₃), 7.55 (d, J=6.11 Hz, 1H, H-5), 7.63 (d, J=5.87 Hz, 1H, H-6), 7.99 (t, J=5.50 Hz, 1H, —NH—CH₂); ¹⁹F NMR (376 MHz, DMSO-d₆): δ−64.67 (t, J=11.58 Hz, 3F. CF₃—).

### Step 1. 2-Isopropyl-6-methyl-3H-thieno[2,3-d]pyrimidin-4-one

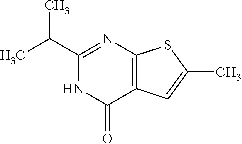

A dark red mixture of 2-amino-3-ethoxycarbonyl-5-methylthiophene (1.04 g, 5.6 mmol) and isobutyronitrile (0.7 mL, 7.8 mmol) was treated with 4.0 M HCl in 1,4-dioxane (3 mL) at room temp. The reaction mixture quickly turned to a thick yellow-orange paste. An additional 2 mL 4N HCl in 1,4-dioxane was added after 30 min and the reaction mixture was heated at 50° C. After 2 h an additional 2 mL of 4.0 N HCl in 1,4-dioxane was added and the mixture was heated at 110° C. overnight. The solvent was evaporated in vacuo to afford a brown-red solid. The solid was dissolved in acetonitrile (20 mL) and cooled to room temperature to afford a crystalline precipitate. The solid was filtered, washed with acetonitrile (2×10 mL) and dried in vacuo to give a pale yellow crystalline solid (754.3 mg, yield 65%). LC-MS analysis of the solid showed the desired product with a purity >98% and the desired product's mass: m/z 209 (M+H), m/z 231 (M+Na) and m/z 439 (2M+Na); Calcd for C₁₀H₁₂N₂OS=208.28.

### Step 2. 4-Chloro-2-isopropyl-6-methyl-thieno[2,3-d]pyrimidine

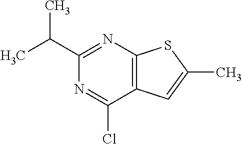

A suspension of 2-isopropyl-6-methyl-3H-thieno[2,3-d]pyrimidin-4-one (754.3 mg, 3.62 mmol) in phosphorus oxychloride (4 mL, 43.0 mmol) was heated at refluxing conditions. Within 15 min an orange solution was obtained. The solvent was evaporated in vacuo to afford an orange viscous liquid. The liquid was poured onto crushed ice-water to give a cream gummy solid. The mixture was neutralized with a saturated NaHCO₃ solution to give a cream precipitate. The precipitate was extracted with ethyl acetate (2×25 mL), the organic and aqueous layers were separated, the organic layer was washed with brine (1×25 mL) and dried over anhydrous Na₂SO₄, filtered and evaporated in vacuo to afford a yellow viscous liquid which solidified partially to a yellow crystalline solid (780.4 mg). The crude product was purified by silica-gel flash chromatography using 0 to 20% EtOAc in hexanes to afford a colorless crystalline solid (755.0 mg, yield 92%). LC-MS analysis of the solid showed the desired product with a purity >98% and the desired product's mass: m/z 227 (^{35Cl}M+H), m/z 229 (^{37Cl}M+H); C₁₀H₁₁ClN₂OS=226.73 ¹H NMR (400 MHz, DMSO-d₆): δ 1.30 (d, J=7.0 Hz, 6H, (CH₃)₂CH—), 2.63 (d, J=1.22 Hz, 3H, CH₃—), 3.19 (dt, J=13.88 and 6.88 Hz, 1H, (CH₃)₂CH—), 7.23 (q, J=1.22 Hz, 1H, H5).

### N-Cyclopropyl-2-isopropyl-6-methyl-thieno[2,3-d]pyrimidin-4-amine (1061)

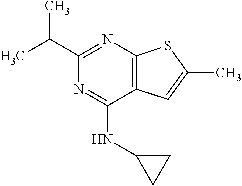 [SMILES: Cc1cc2c(NC3CC3)nc(C(C)C)nc2s1]

A solution of 4-chloro-2-isopropyl-6-methyl-thieno[2,3-d]pyrimidine (69.0 mg, 0.30 mmol), DIEA (70 μL, 0.42 mmol) and cyclopropylamine (66.0 μL, 0.94 mmol) in 1,4-dioxane (1 mL) was heated at 140° C. in a microwave reactor for 3 h to give a yellow-orange solution and the solvent was evaporated in vacuo to afford an orange-red viscous residue. The crude residue was purified by reverse-phase preparative HPLC to afford a colorless to light tan crystalline residue (102.0 mg). The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless to pale tan crystalline solid (64.2 mg, yield 85%); LC-MS analysis of the solid showed the desired product with a purity >98% and the desired product's mass: m/z 248 (M+H); Calcd for C₁₃H₁₇N₃S=247.36. ¹H NMR (400 MHz, DMSO-d₆): δ 0.53-0.59 (m, 2H, —CH₂—), 0.74-0.80 (m, 2H, —CH₂—), 1.25 (d, J=7.00 Hz, 6H, (CH₃)₂CH—), 2.49 (d, J=1.22 Hz, 3H, C₃—), 2.94 (sept, J=6.8 Hz, 1H, (CH₃)₂CH—), 2.99 (sept, J=3.7 Hz, 1H, —CH—), 7.20 (s, 1H, —NH—), 7.65 (d, J=3.5 Hz, 1H).

### Step 1. 6-Methyl-2-phenyl-3H-thieno[2,3-d]pyrimidin-4-one

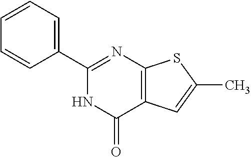 [SMILES: Cc1cc2c(=O)[nH]c(-c3ccccc3)nc2s1]

A dark red mixture of 2-amino-3-ethoxycarbonyl-5-methylthiophene (645.0 mg, 3.48 mmol) and benzonitrile (500 μL, 4.90 mmol) was treated with 4.0 M HCl in 1,4-dioxane (3 mL, 12.0 mmol) at room temp. The reaction mixture quickly turned to a thick orange-red paste. An additional 2 mL 4 M HCl in 1,4-dioxane was added after 30 min and the reaction mixture was heated at 50° C. After heating at 50° C. for 1.5 h, the reaction mixture was heated at 110° C. to give a thick orange-brown paste with 2 h. Another 3 mL of 4.0 M HCl in 1,4-dioxane was added and the mixture was heated at 110° C. overnight. to give a cream precipitate. The solvent was evaporated in vacuo to afford a dirty brown-red residue. The solid was triturated with acetonitrile (10 mL) to give a cream precipitate, the solid was filtered, washed with acetonitrile (2×10 mL) and dried in vacuo to give an almost colorless crystalline powder (152.4 mg, yield 18%). LC-MS analysis of the solid showed the desired product with a purity >98% and the desired product's mass: m/z 243 (M+H), m/z 265 (M+Na) and m/z 507 (2M+Na); Calcd for C₁₃H₁₀N₂OS=242.30.

### Step 2. 4-Chloro-6-methyl-2-phenyl-thieno[2,3-d]pyrimidine

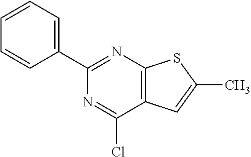 [SMILES: Cc1cc2c(Cl)nc(-c3ccccc3)nc2s1]

A suspension of 6-methyl-2-phenyl-3H-thieno[2,3-d]pyrimidin-4-one (153.0 mg, 0.63 mmol) in phosphorus oxychloride (1 mL, 10.7 mmol) was heated at refluxing conditions for 30 min. The solvent was evaporated in vacuo to afford a light orange viscous liquid. The liquid was poured onto crushed ice-water to give a cream gummy solid and the mixture was neutralized with a saturated NaHCO₃ solution to give a cream precipitate. The precipitate was extracted with ethyl acetate (2×25 mL), the organic and aqueous layers were separated, the organic layer was washed with brine (1×25 mL) and dried over anhydrous Na₂SO₄, filtered and evaporated in vacuo to afford a cream solid (164.8 mg, yield 100%). LC-MS analysis of the solid showed the desired product with a purity >98% and the desired product's mass: m/z 261 (^{35Cl}M+H), and m/z 263 (^{37Cl}M+Na); Calcd for C₁₃H₉ClN2S: 260.74. ¹H NMR (400 MHz, DMSO-d₆): δ 2.66 (s, 3H, —CH₃), 7.30 (d, J=1.22 Hz, 1H, H-5), 7.50-7.59 (m, 3H), 8.39 (dd, J=6.60 and 2.93 HZ, 2H).

### N-Cyclopropyl-6-methyl-2-phenyl-thieno[2,3-d]pyrimidin-4-amine (1062)

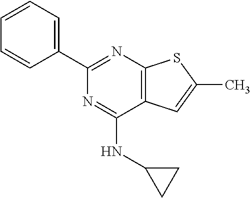 [SMILES: Cc1cc2c(NC3CC3)nc(-c3ccccc3)nc2s1]

A solution of 4-chloro-6-methyl-2-phenyl-thieno[2,3-d]pyrimidine (51.7 mg, 0.20 mmol), DIEA (45 μL, 0.26 mmol) and cyclopropylamine (43 μL, 0.62 mmol) in 1,4-dioxane (1 mL) was heated at 140° C. in a microwave reactor for 3 h to give a pale yellow solution and the solvent was evaporated in vacuo to afford a pale yellow-orange viscous liquid. The crude residue was purified by reverse-phase preparative HPLC to afford a colorless crystalline residue (66.1 mg). The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless to cream crystalline solid (52.2 mg, yield 94%). LC-MS analysis of the solid showed the desired product with a purity >98% and the desired product's mass: m/z 282 (M+H); Calcd for C₁₆H₁₅N₃S=281.38. ¹H NMR (400 MHz, DMSO-d₆): δ 0.56-0.69 (m, 2H, —CH₂—), 0.79-0.91 (m, 2H, —CH₂—), 2.53 (d, J=1.22 Hz, 3H, 6-CH₃—), 3.10 (td, J=7.15 and 3.55 Hz, 1H, —CH— (cypyl)), 7.28 (s, 1H, H-5), 7.39-7.54 (m, 3H), 7.88 (d, J=3.42 Hz, 1H, —NH—), 8.37-8.47 (m, 2H).

### Step 1. 2-[(4-Fluorophenyl)methyl]-6-methyl-3H-thieno[2,3-d]pyrimidin-4-one

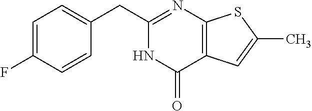 [SMILES: Cc1cc2c(=O)[nH]c(Cc3ccc(F)cc3)nc2s1]

A dark red mixture of 2-amino-3-ethoxycarbonyl-5-methylthiophene (1.03 g, 5.56 mmol) and 4-fluorophenylacetonitrile (800 μL, 6.67 mmol) was treated with 4.0 M HCl in 1,4-dioxane (3 mL, 12.0 mmol) at room temp. The reaction mixture quickly turned to a thick yellow-orange paste. An additional 2 mL 4N HCl in 1,4-dioxane was added after 30 min and the reaction mixture was heated at 50° C. to give a red solution. After heating at 50° C. for 4 h, the reaction mixture was heated at 110° C. to give a thick yellow paste within 2 h. Another 2 mL of 4.0 N HCl in 1,4-dioxane was added and the mixture was heated at 110° C. overnight. The solvent was evaporated in vacuo to afford a light brown solid. The solid was triturated with acetonitrile (10 mL) to give a cream precipitate. The solid was filtered, washed with acetonitrile (2×10 mL) and dried in vacuo to give an almost colorless powder (1.40 g, yield 92%). LC-MS analysis of the solid showed the desired product with a purity >98% and the desired product's mass: m/z 275 (M+H), m/z 297 (M+Na) and m/z 571 (2M+Na); Calcd for C₁₄H11FN₂OS=274.31.

### Step 2. 4-Chloro-2-[(4-fluorophenyl)methyl]-6-methyl-thieno[2,3-d]pyrimidine

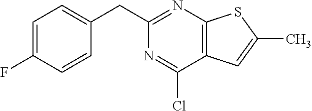 [SMILES: Cc1cc2c(Cl)nc(Cc3ccc(F)cc3)nc2s1]

A suspension of 2-[(4-fluorophenyl)methyl]-6-methyl-3H-thieno[2,3-d]pyrimidin-4-one (1.054 g, 3.84 mmol) in phosphorus oxychloride (5 mL, 54.0 mmol) was heated at refluxing conditions overnight. The solvent was evaporated in vacuo to afford an orange viscous liquid. The liquid was poured onto crushed ice-water to give an orange precipitate. The mixture was neutralized with a saturated NaHCO₃ solution to give a yellow-orange precipitate. The precipitate was extracted with ethyl acetate (2×25 mL), the organic and aqueous layers were separated, the organic layer was washed with brine (1×25 mL) and dried over anhydrous Na₂SO₄, filtered and evaporated in vacuo to afford a yellow crystalline solid. The crude product was purified by silica-gel flash chromatography using EtOAc in hexanes to afford a colorless microcrystalline solid (761.0 mg, yield 66%). LC-MS analysis of the solid showed the desired product with a purity >98% and the desired product's mass: m/z 293 (^{35Cl}M+H), m/z 295 (^{37Cl}M+H); Calcd for C₁₄H₁₀ClFN₂S=292.76. ¹H NMR (400 MHz, DMSO-d₆): δ 2.63 (d, J=1.22 Hz, 3H, 6-CH₃—), 4.27 (s, 2H, —CH₂—), 7.08-7.18 (m, 2H), 7.24 (d, J=1.22 Hz, 1H, H5), 7.30-7.42 (m, 2H); ¹⁹F NMR (376 MHz, DMSO-d₆): δ−116.48 (s), 4-F-benzyl-).

### N-Cyclopropyl-2-[(4-fluorophenyl)methyl]-6-methyl-thieno[2,3-d]pyrimidin-4-amine (1063)

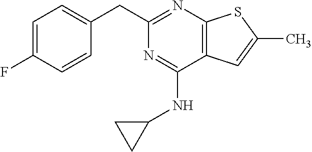 [SMILES: Cc1cc2c(NC3CC3)nc(Cc3ccc(F)cc3)nc2s1]

A solution of a mixture of 4-chloro-2-[(4-fluorophenyl)methyl]-6-methyl-thieno[2,3-d]pyrimidine (55.7 mg, 0.19 mmol), DIEA (50 μL, 0.29 mmol) and cyclopropylamine (40 μL, 0.57 mmol) in 1,4-dioxane (1.2 mL) was heated at 140° C. in a microwave reactor for 3 h to give a pale yellow solution with a trace of a colorless suspension. The crude residue was purified by reverse-phase preparative HPLC to afford a colorless crystalline solid (75.0 mg). The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a cream to colorless solid (47.8 mg, yield 80%). LC-MS of the solid showed the desired product with a purity >98% and the desired product's mass: m/z 314 (M+H); Calcd for C₁₇H₁₆FN₃S=313.39. ¹H NMR (400 MHz, DMSO-d₆): δ 0.48-0.59 (m, 2H, —CH₂—), 0.72-0.82 (m, 2H, —CH₂—), 2.48 (d, J=0.98 Hz, 3H, 6-CH₃—), 2.96 (td, J=7.09 and 3.67 Hz, 1H, —CH— (cypyl)), 3.98 (s, 2H, —CH₂—), 7.05-7.13 (m/tt, 2H), 7.20 (brs, 1H), 7.40 (dd, J=8.56 and 5.87 Hz, 2H), 7.76 (d, J=3.18 HZ, 1H). ¹⁹F NMR (376 MHz, DMSO-d₆): δ −117.30 (sept, J=5.5 Hz, 4-F—).

### Step 1. 2-Ethyl-6-(trifluoromethyl)-3H-thieno[2,3-d]pyrimidin-4-one

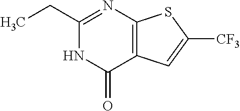 [SMILES: CCc1nc2sc(C(F)(F)F)cc2c(=O)[nH]1]

A pale yellow solution of ethyl 2-amino-5-(trifluoromethyl)thiophene-3-carboxylate (256.6 mg, 1.14 mmol) and propionitrile (150 μL, 2.1 mmol) was treated with 4.0 M HCl in 1,4-dioxane (3 mL, 12 mmol) at room temperature. The reaction mixture quickly turned to a thick yellow suspension. After stirring for 30 min at room temperature, the reaction mixture was heated at 50° C. and then the reaction mixture was warmed to 110° C. to give a darker yellow solution. Another 3 mL of 4.0 N HCl in 1,4-dioxane was added after 5 min and the reaction mixture was heated at 110° C. overnight. A fresh batch of propionitrile (150 μL) and 4.0 M HCl in 1,4-dioxane (3 mL) was added and the reaction mixture was heated at 110° C. for 7 h to give a darker yellow solution/suspension. The solvent was evaporated in vacuo to afford a cream-yellow crystalline solid. The solid was dissolved in acetonitrile (20 mL) and cooled to room temperature to afford a colorless to cream crystalline precipitate. The solid was filtered, washed with acetonitrile (2×5 mL) and dried in vacuo to give a cream crystalline solid (140.6 mg, yield 50%). LC-MS analysis of the solid showed the desired product with a purity >98% and the desired product's mass: m/z 249 (M+H), and m/z 271 (M+Na); C₉H₇F₃N₂OS: 248.22. ¹H NMR (400 MHz, DMSO-d₆): δ 1.23 (t, J=7.58 Hz, 3H, CH₃CH₂—), 2.68 (q, J=7.58 Hz, 2H, CH₃CH₂—), 7.19 (brs, 1H, —CONH—), 7.78-8.01 (m/q, 1H, H-5); ¹⁹F NMR (376 MHz, DMSO-d₆): δ −54.71 (s), 6-CF₃—.

### Step 2. 4-Chloro-2-ethyl-6-(trifluoromethyl)thieno[2,3-d]pyrimidine

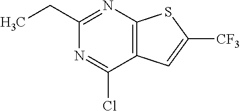 [SMILES: CCc1nc(Cl)c2cc(C(F)(F)F)sc2n1]

A suspension of 2-ethyl-6-(trifluoromethyl)-3H-thieno[2,3-d]pyrimidin-4-one (124.0 mg, 0.5 mmol) in phosphorus oxychloride (2.5 mL, 27 mmol) was heated at refluxing conditions to give a. colorless solution within 15 min. The reaction mixture was cooled to room temperature and evaporated in vacuo to give a pale yellow viscous liquid. The liquid was treated with crushed ice-water to give a cream-yellow precipitate. The mixture was neutralized with a saturated NaHCO₃ (1×5 mL) solution and the mixture was extracted with ethyl acetate (2×25 mL), the aqueous and the organic layers were separated, the organic layer was washed with brine (1×25 mL) and dried over anhydrous Na₂SO₄, filtered and evaporated in vacuo to afford a pale yellow liquid. The crude product was purified by silica-gel flash chromatography to afford a colorless liquid (107.4 mg, yield 81%). LC-MS analysis of the liquid showed the desired product with a purity >98% and the desired product's mass: m/z 267 (^{35Cl}M+H) and m/z 269 (^{37Cl}M+H); Calcd for C₉H₆ClF₃N₂S: 266.67. ¹H NMR (400 MHz, DMSO-d₆): δ 1.33 (t, J=7.58 Hz, 3H, CH₃CH₂—), 3.04 (q, J=7.58 Hz, 2H, CH₃CH₂—), 8.28 (q, J_{H-F}=1.22 Hz, 1H, H-5); ¹⁹F NMR (376 MHz, DMSO-d₆): δ −55.65 (s).

### N-Cyclopropyl-2-ethyl-6-(trifluoromethyl)thieno[2,3-d]pyrimidin-4-amine (1177)

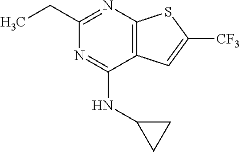 [SMILES: CCc1nc(NC2CC2)c2cc(C(F)(F)F)sc2n1]

A solution of 4-chloro-2-ethyl-6-(trifluoromethyl)thieno[2,3-d]pyrimidine (60.0 mg, 0.225 mmol), DIEA (60 μL, 0.35 mmol) and cyclopropylamine (50 μL, 0.72 mmol) in 1,4-dioxane (1.5 mL) was heated at 140° C. in a microwave reactor for 3 h to give a pale yellow solution with colorless suspension. The solvent was evaporated in vacuo to afford a yellow glassy residue (91.0 mg). The crude residue was purified by reverse-phase preparative HPLC to afford a colorless residue. LC-MS analysis of the residue shows the desired product at rt 2.03 min with a purity >98%. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless to cream crystalline solid (55.0 mg, yield 85%). LC-MS analysis of the freebase product showed the desired product with a purity >98% and the desired product's mass: m/z 288 (M+H); Calcd for CH₁₂H₁₂F₃N₃S=287.30. ¹H NMR (400 MHz, DMSO-d₆): δ 0.59 (brs, 2H, —CH₂—), 0.79-0.86 (m, 2H, —CH₂—), 1.27 (t, J=7.46 Hz, 3H, CH₃CH₂—), 2.76 (q, J=7.58 Hz, 2H, CH₃CH₂—), 3.05 (td, J=7.21 Hz and 3.67 Hz, 1H, —NH—CH-(cypyl), 8.20 (brs, 1H, H-5), 8.23 (brs, 1H, —NH—); ¹⁹F NMR (376 MHz, DMSO-d₆): δ −54.99 (s).

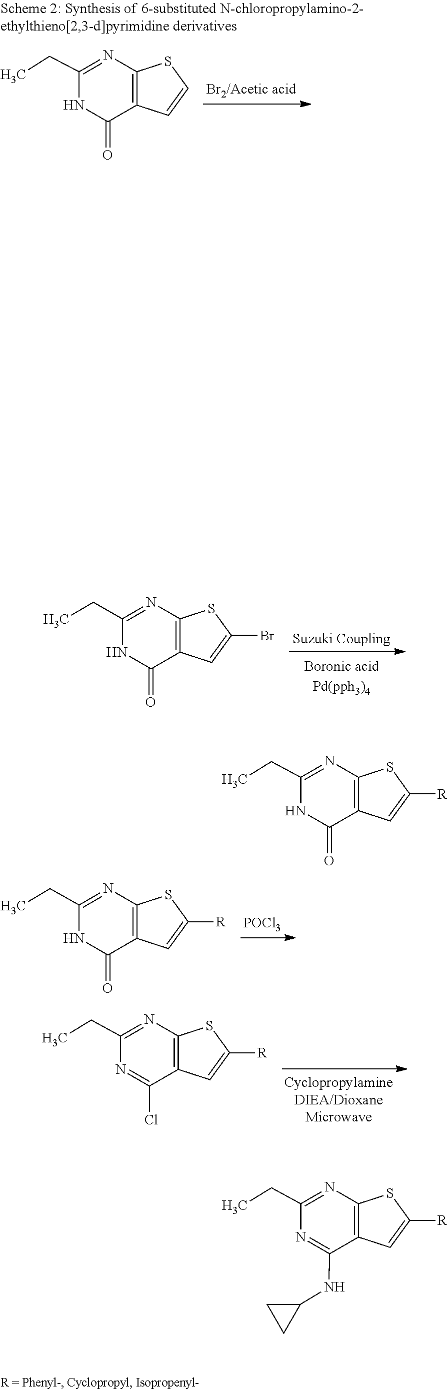

Scheme 2 shows a general method for the Suzuki-Miyura type cross coupling of a 6-bromo-thienopyrimidine intermediate with the corresponding boronic acids. This procedure afforded the corresponding 6-substituted thienopyrimidinones which were converted to the corresponding 4-chloro-thienopyrimidines by the reaction of phosphorus oxychloride. The 4-aminoalkyl derivatives were synthesized by the reactions of the appropriate 4-chloro-thienopyrimidines with cyclopropylamine in the presence of a tert-amine and the microwave heating methodology.

### Step 1. 6-Bromo-2-ethyl-3H-thieno[2,3-d]pyrimidin-4-one

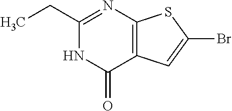

To a solution of 2-ethyl-3H-thieno[2,3-d]pyrimidin-4-one (136.5 mg, 0.76 mmol) in glacial acetic acid (5 mL) was added bromine (50 μL, 0.98 mmol) at room temperature and the reaction mixture was stirred at room temperature for 30 min to give an orange solid. The solvent was evaporated in vacuo and the solid was suspended in water (25 mL) and stirred at room temperature for 5 min and the solid was filtered, washed with water (1×10 mL) and dried in vacuo to give a colorless solid (168.4 mg, yield 86%). LC-MS analysis of the crude product showed the desired product with a purity >95% and the desired product's mass: m/z 259 (^{79Br}M+H), m/z 261 (^{81Br}M+H), m/z 281 (^{79Br}M+Na), and m/z 283 (^{81Br}M+Na); Calcd for C₈H₇BrN₂OS=259.12. The crude product will be used as such for the cross-coupling reactions.

### Step 2. 6-Bromo-4-chloro-2-ethyl-thieno[2,3-d]pyrimidine

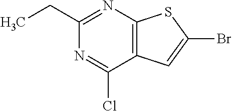

A suspension of 6-bromo-2-ethyl-3H-thieno[2,3-d]pyrimidin-4-one (82.8 mg, 0.32 mmol) in phosphorus oxychloride (1 mL) was heated at refluxing conditions for 1 h. The solvent was evaporated in vacuo to afford a light orange-brown viscous liquid. The liquid was poured onto crushed ice-water to give a cream precipitate and the mixture was neutralized with a saturated NaHCO₃ solution. The precipitate was extracted with ethyl acetate (2×25 mL), the aqueous and the organic layers were separated, the organic layer was washed with brine (1×25 mL) and dried over anhydrous Na₂SO₄, filtered and evaporated in vacuo to afford a cream crystalline solid. The crude product was purified by silica-gel flash chromatography using 0 to 20% EtOAc in hexanes to afford a colorless viscous liquid which solidified to a colorless crystalline solid (84.8 mg, yield 96%). LC-MS analysis of the solid showed the desired product with a purity >98% and the desired product's mass: m/z 277 (^{35Cl,79Br}M+H), m/z 279 (^{35Cl,81Br/37Cl,79Br}M+H), m/z 281 (^{37Cl,81Br}M+H); Calcd for C₈H₆BrClN₂S=277.56. ¹H NMR (400 MHz, DMSO-d₆): δ 1.30 (t, J=7.58 Hz, 3H, CH₃CH₂—), 2.96 (q, J=7.58 Hz, 2H, CH₃CH₂—), 7.81 (s, 1H, H-5).

### 6-Bromo-N-cyclopropyl-2-ethyl-thieno[2,3-d]pyrimidin-4-amine (1146)

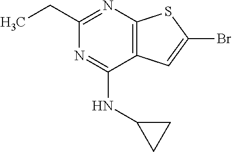 [SMILES: CCc1nc(NC2CC2)c2cc(Br)sc2n1]

A solution of 6-bromo-4-chloro-2-ethyl-thieno[2,3-d]pyrimidine (46.0 mg, 0.17 mmol), DIEA (40 μL, 0.24 mmol) and cyclopropylamine (35 μL, 0.50 mmol) in 1,4-dioxane (1.5 mL) was heated at 140° C. in a microwave reactor for 2 h to give a pale yellow suspension and the solvent was evaporated in vacuo to afford an orange glassy residue. The crude residue was purified by reverse-phase preparative HPLC to afford a colorless glassy residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (43.0 mg, yield 87%). LC-MS analysis of the freebase product showed the desired product with a purity >98% and the desired product's mass: m/z 298 (^{79Br}M+H), and m/z 300 (^{81Br}M+H); Calcd for C₁₁H₁₂BrN₃S=298.20. ¹H NMR (400 MHz, DMSO-d₆): δ 0.50-0.62 (m, 2H, —CH₂—), 0.71-0.84 (m, 2H, —CH₂—), 1.24 (t, J=7.58 Hz, 3H, CH₃—CH₂—), 2.70 (q, J=7.58 Hz, 2H, CH₃—CH₂—), 2.98 (tq, J=7.24 and 3.73 Hz, 1H, —CH-(cypyl)), 7.71 (s, 1H, H-5), 7.86 (brs 1H, —NH—).

### Step 1. 2-Ethyl-6-phenyl-3H-thieno[2,3-d]pyrimidin-4-one

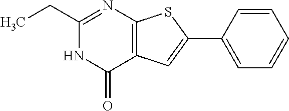 [SMILES: CCc1nc2sc(-c3ccccc3)cc2c(=O)[nH]1]

A suspension of a mixture of 6-bromo-2-ethyl-3H-thieno[2,3-d]pyrimidin-4-one (78.8 mg, 0.31 mmol), phenylboronic acid (74.2 mg, 0.61 mmol) and Pd(PPh₃)₄ (35.0 mg, 0.03 mmol) in DMF (2.0 mL) was stirred at room temperature under nitrogen atmosphere. A de-gassed solution of cesium carbonate (198.0 mg, 0.61 mmol) in water (1.0 mL) was added and the reaction mixture was heated at 80° C. under nitrogen atmosphere for 4.5 h. The solvent was evaporated in vacuo to afford a light orange-brown residue. The residue was partitioned between water (25 mL) and ethyl acetate acetate (25 mL), the aqueous and the organic layers were separated, the organic layer was washed with brine (1×25 mL) and dried over anhydrous Na₂SO₄, filtered and evaporated in vacuo to afford a dirty orange solid. The crude product was triturated with acetonitrile (10 mL) and the precipitated solid was stirred at room temperature for 5 min, filtered and washed with acetonitrile (1×10 mL) to give a light tan to beige solid.

The precipitated solid was dissolved in hot methanol, filtered and evaporated in vacuo to afford a dirty yellow crystalline solid (59.6 mg, yield 77%). LC-MS analysis of the solid showed the desired product with a purity >95% and the desired product's mass: m/z 257 (M+H), m/z 279 (M+Na) and m/z 535 (2M+Na); Calcd for C₁₄H₁₂N₂OS=256.32.

### Step 2. 4-Chloro-2-ethyl-6-phenyl-thieno[2,3-d]pyrimidine

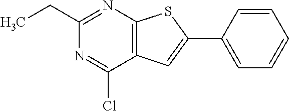 [SMILES: CCc1nc(Cl)c2cc(-c3ccccc3)sc2n1]

A suspension of 2-ethyl-6-phenyl-3H-thieno[2,3-d]pyrimidin-4-one (59.6 mg, 0.24 mmol) in phosphorus oxychloride (1.0 mL, 10.73 mmol) was heated at refluxing conditions for 1 h. The reaction mixture was cooled to room temperature and evaporated in vacuo, to give a cream solid and solid was treated with crushed ice-water to give a cream precipitate. The mixture was neutralized with a saturated NaHCO₃ (1×5 mL) solution and the mixture was extracted with ethyl acetate (2×25 mL), the aqueous and the organic layers were separated, the organic layer was washed with brine (1×25 mL) and dried over anhydrous Na₂SO₄, filtered and evaporated in vacuo to afford a cream solid. The crude product was purified by silica-gel flash chromatography to afford a colorless crystalline solid (63.0 mg, yield 99%). LC-MS analysis of the solid showed the desired product with a purity >98% and the desired product's mass: m/z 275 (^{35Cl}M+H), and m/z 277 (^{37Cl}M+H); Calcd for C₁₄H₁₁ClN₂S=274.77. ¹H NMR (400 MHz, DMSO-d₆): δ 1.33 (t, J=7.58 Hz, 3H, CH₃CH₂—), 2.99 (q, J=7.58 Hz, 2H, CH₃CH₂—), 7.45-7.56 (m, 3H, Ph-H), 7.88-7.90 (m, 1H, Ph-H), 7.90-7.92 (m, 1H, Ph-H), 7.93 (s, 1H, H-5).

### N-Cyclopropyl-2-ethyl-6-phenyl-thieno[2,3-d]pyrimidin-4-amine (1150)

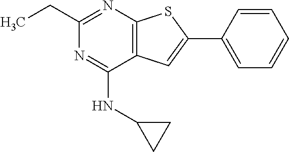 [SMILES: CCc1nc(NC2CC2)c2cc(-c3ccccc3)sc2n1]

A solution of 4-chloro-2-ethyl-6-phenyl-thieno[2,3-d]pyrimidine (53.0 mg, 0.19 mmol), DIEA (50 μL, 0.29 mmol) and cyclopropylamine (40 μL, 0.57 mmol) in 1,4-dioxane (2.0 mL) was heated at 140° C. in a microwave reactor for 5 h to give a pale yellow suspension. The solvent was evaporated in vacuo to afford an orange glassy residue. The crude residue was purified by reverse-phase preparative HPLC to afford a colorless solid. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (49.0 mg; yield 86%). LC-MS analysis of the freebase product showed the desired product with a purity >98% and the desired product's mass: m/z 296 (M+H); Calcd for C₁₇H₁₇N₃S=295.40. ¹H NMR (400 MHz, DMSO-d₆): δ 0.56-0.63 (m, 2H, —CH₂—), 0.76-0.86 (m, 2H, —CH₂—), 1.27 (t, J=7.58 Hz, 3H, CH₃CH₂—), 2.74 (q, J=7.58 Hz, 2H, CH₃CH₂—), 3.04 (td, J=7.15 and 3.55 Hz, 1H, —CH— (cypyl)), 7.33-7.40 (m, 1H), 7.48 (t, J=7.70 Hz, 2H), 7.64 (d, J=7.58 Hz, 2H), 7.88 (d, J=2.0.69 Hz, 1H, —NH—), 7.93 (s, 1H, H-5).

### Step 1. 6-Cyclopropyl-2-ethyl-3H-thieno[2,3-d]pyrimidin-4-one

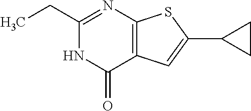

A suspension of a mixture of 6-bromo-2-ethyl-3H-thieno[2,3-d]pyrimidin-4-one (105.6 mg, 0.41 mmol), cyclopropylboronic acid (59.0 mg, 0.69 mmol) and Pd(PPh₃)₄ (47.5 mg, 0.041) in 1,4-dioxane (2.0 mL) was stirred at room temperature under nitrogen atmosphere. A degassed solution of sodium carbonate (170.0 mg, 1.23 mmol) in water (1.0 mL) was added and the reaction mixture was heated in a sand-bath at 120° C. under nitrogen atmosphere overnight. The crude residue was purified by reverse-phase preparative HPLC to afford the desired product: 6-cyclopropyl-2-ethyl-3H-thieno[2,3-d]pyrimidin-4-one as a colorless to pale yellow microcrystalline solid (24.6 mg, yield 27%). LC-MS analysis of the solid showed the desired product with a purity >95% and the desired product's mass: m/z 221 (M+H) and m/z 243 (M+Na); Calcd for C₁₁H₁₂N₂OS=220.209.

### Step 2. 4-Chloro-6-cyclopropyl-2-ethyl-thieno[2,3-d]pyrimidine

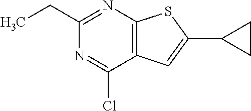

A suspension of 6-cyclopropyl-2-ethyl-3H-thieno[2,3-d]pyrimidin-4-one (84.0 mg, 0.38) in phosphorus oxychloride (1.0 mL, 10.73 mmol) was heated at refluxing conditions for 1 h. The reaction mixture was cooled to room temperature and evaporated in vacuo to give a dirty yellow viscous liquid. The liquid was treated with crushed ice-water to give a cream-yellow precipitate. The mixture was neutralized with a saturated NaHCO₃ (1×5 mL) solution and the mixture was extracted with ethyl acetate (2×25 mL), the aqueous and the organic layers were separated, the organic layer was washed with brine (1×25 mL) and dried over anhydrous Na₂SO₄, filtered and evaporated in vacuo to afford a dirty yellow gummy residue. The crude product was purified by silica-gel flash chromatography to afford a colorless liquid (72.00 mg, yield 79%). LC-MS analysis of the liquid showed the desired product at rt 2.68 min with a purity >98% and the desired product's mass: m/z 239 (^{35Cl}M+H) and m/z 241 (^{37Cl}M+H); Calcd for C₁₁H₁₁ClN₂S=239.73. ¹H NMR (400 MHz, DMSO-d₆): δ 0.88-0.96 (m, 2H, —CH₂—), 1.10-1.21 (m, 2H, —CH₂—), 1.29 (t, J=7.58 Hz, 3H, CH₃CH₂—), 2.29-2.42 (m, 1H, —CH— (cypyl)), 2.94 (q, J=7.58 Hz, 2H, CH₃CH₂—), 7.18 (s, 1H, H-5).

### N,6-Dicyclopropyl-2-ethyl-thieno[2,3-d]pyrimidin-4-amine (1161)

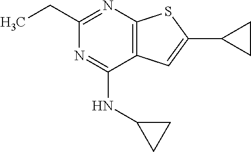 [SMILES: CCc1nc(NC2CC2)c2cc(C3CC3)sc2n1]

A solution of 4-chloro-6-cyclopropyl-2-ethyl-thieno[2,3-d]pyrimidine (55.0 mg, 0.23 mmol), DIEA (57 μL, 0.34 mmol) and cyclopropylamine (50 μL, 0.72 mmol) in 1,4-dioxane (2.0 mL) was heated at 140° C. in a microwave reactor until a >95% conversion was achieved (total 10 h). The solvent was evaporated in vacuo to afford an orange glassy residue. The crude residue was purified by reverse-phase preparative HPLC to afford a pale yellow glassy residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a pale yellow glassy residue. The solid was dissolved in a mixture of water/acetonitrile and lyophilized to afford a cream lyophilized powder (35.0 mg; yield 59%). LC-MS analysis of the freebase product showed the desired product with a purity >98% and the desired product's mass: m/z 260 (M+H; Calcd for C₁₄H₁₇N₃S=259.37. ¹H NMR (400 MHz, DMSO-d₆): δ 0.51-0.57 (m, 2H, —CH₂—), 0.67-0.72 (m, 2H, —CH₂—), 0.73-0.79 (m, 2H, —CH₂—), 1.00-1.07 (m, 2H, —CH₂—), 1.23 (t, J=7.58 Hz, 3H, CH₃—CH₃—), 2.09-2.20 (m, 1H, —CH— (cypyl)), 2.69 (q, J=7.58 Hz, 2H, CH₃—CH₂—), 2.97 (td, J=7.15 and 3.30 Hz, 1H, —CH— (cypyl)), 7.19 (s, 1H, 5-H), 7.61 (d, J=3.42 Hz, 1H, —NH—).

### Step 1. 2-Ethyl-6-isopropenyl-3H-thieno[2,3-d]pyrimidin-4-one

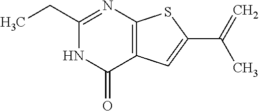

A suspension of a mixture of 6-bromo-2-ethyl-3H-thieno[2,3-d]pyrimidin-4-one (100.5 mg, 0.39 mmol), potassium isopropenyltrifluoroborate (119.42 mg, 0.775 mmol) and Pd(PPh₃)₄ (49.0 mg, 0.042 mmol) in 1,4-dioxane (3.0 mL) was stirred at room temperature under nitrogen atmosphere to give a yellow-orange solution. A de-gassed solution of cesium carbonate (398.0 mg, 1.23 mmol) in water (1.0 mL) was added and the reaction mixture was heated at 90° C. for 2 h under nitrogen atmosphere. The reaction mixture was cooled to room temperature and the solvent was evaporated in vacuo to afford a dark orange-brown residue. The residue was partitioned between water (25 mL) and ethyl acetate (25 mL), the aqueous and the organic layers were separated, the organic layer was washed with brine (1×25 mL) and dried over anhydrous Na₂SO₄, filtered and evaporated in vacuo to afford an orange-brown solid. The crude product was triturated with acetonitrile (10 mL) and the precipitated solid was stirred at room temperature for 5 min, filtered and washed with acetonitrile (1×10 mL) to give a cream crystalline solid (72.5 mg, yield 85%). LC-MS analysis solid showed the desired product with a purity >95% and the desired product's mass: m/z 221 (M+H), and m/z 243 (M+Na); Calcd for C₁₁H₁₂N₂OS=220.29

### Step 2. 4-Chloro-2-ethyl-6-isopropenyl-thieno[2,3-d]pyrimidine

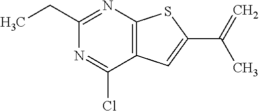

A suspension of 2-ethyl-6-isopropenyl-3H-thieno[2,3-d]pyrimidin-4-one (72.5 mg, 0.33 mmol) in phosphorus oxychloride (1.5 mL, 16.1 mmol) was heated at refluxing conditions for 1 h. The reaction mixture was cooled to room temperature and evaporated in vacuo to give a pale yellow viscous liquid. The liquid was treated with crushed ice-water to give a cream precipitate. The mixture was neutralized with a saturated NaHCO₃ (1×5 mL) solution and the mixture was extracted with ethyl acetate (2×25 mL), the aqueous and the organic layers were separated, the organic layer was washed with brine (1×25 mL) and dried over anhydrous Na₂SO₄, filtered and evaporated in vacuo to afford a pale yellow viscous liquid. The crude product was purified by silica-gel flash chromatography to afford a colorless crystalline solid (75.6 mg, yield 96%).

LC-MS analysis of the solid showed the desired product with a purity >98% and the desired product's mass: m/z 239 (^{35Cl}M+H) and m/z 241 (^{37Cl}M+H); Calcd for C₁₁H₁₁ClN₂S=238.73. ¹H NMR (400 MHz, DMSO-d₆): δ 1.31 (t, J=7.58 Hz, 3H, CH₃CH₂—), 2.22 (s, 3H, CH₃—), 2.97 (q, J=7.58 Hz, 2H, CH₃CH₂—), 5.38 (d, J=1.22 Hz, 1H, ═CHH), 5.59 (s, 1H, ═CHH), 7.41 (s, 1H, H-5).

### N-Cyclopropyl-2-ethyl-6-(prop-1-en-2-yl)thieno[2,3-d]pyrimidin-4-amine (1154)

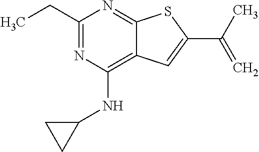 [SMILES: C=C(C)c1cc2c(NC3CC3)nc(CC)nc2s1]

A solution of 4-chloro-2-ethyl-6-isopropenyl-thieno[2,3-d]pyrimidine (58.0 mg, 0.243 mmol), DIEA (60 μL, 0.35 mmol) and cyclopropylamine (50 μL, 0.72 mmol) in 1,4-dioxane (2.0 mL) was heated at 140° C. in a microwave reactor for 3 h to give a pale yellow suspension. The solvent was evaporated in vacuo to afford an orange glassy residue. The crude residue was purified by reverse-phase preparative HPLC to afford a pale yellow glassy residue. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a pale yellow microcrystalline solid. The solid was dissolved in a mixture of water/acetonitrile and lyophilized to afford a cream lyophilized powder (40.0 mg, yield 64%). LC-MS analysis of the freebase product showed the desired product with a purity >97% and the desired product's mass: m/z 260 (M+H); Calcd for C₁₄H₁₇N₃S=259.37. ¹H NMR (400 MHz, DMSO-d₆): δ 0.51-0.63 (m, 2H, —CH₂—), 0.70-0.83 (m, 2H, —CH₂—), 1.25 (t, J=7.58 Hz, 3H, CH₃—CH₂—), 2.13 (s, 3H, CH₃—), 2.71 (q, J=7.58 Hz, 2H, CH₃—CH₂—), 3.02 (td, J=7.21 and 3.91 Hz, 1H, —CH— (cypyl)), 5.13 (s, 1H, ═CHH), 5.32 (s, 1H, ═CHH), 7.54 (s, 1H, 5-H), 7.84 (d, J=3.42 Hz, 1H, —NH—).

### 6-Chloro-N-cyclopropyl-2-ethyl-thieno[2,3-d]pyrimidin-4-amine (1181)

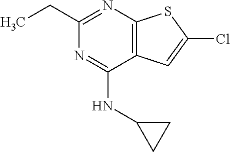 [SMILES: CCc1nc(NC2CC2)c2cc(Cl)sc2n1]

A solution of 4,6-dichloro-2-ethyl-thieno[2,3-d]pyrimidine (53.3 mg, 0.23 mmol), DIEA (64 μL, 0.38 mmol), and cyclopropylamine (54 μL, 0.76 mmol) in 1,4-dioxane (1.5 mL) was heated at 140° C. in a microwave reactor for 3 h to give a pale yellow solution with colorless suspension and the solvent was evaporated in vacuo to afford a yellow glassy residue. The crude residue was purified by reverse-phase preparative HPLC to afford a colorless to pale yellow residue. The residue dissolved in water containing a trace of acetonitrile and lyophilized to afford a colorless to cream lyophilized powder. The lyophilized product was dissolved in acetonitrile containing a trace of methanol and the solution was passed through a SiliaPrep Carbonate (Si—CO₃) 6 mL-1 g cartridge. The filtrate was evaporated in vacuo to afford a colorless to cream crystalline solid (50.5 mg, yield 87%). LC-MS analysis of solid showed the desired product with a purity >98% and the desired product's mass: m/z 254 (^{35Cl}M+H) and m/z 256 (^{37Cl}M+H); Calcd for C₁₁H₁₂ClN₃S=253.75. ¹H NMR (400 MHz, DMSO-d₆): δ 0.51-0.60 (m, 2H, —CH₂—), 0.73-0.83 (m, 2H, —CH₂—), 1.24 (t, J=7.58 Hz, 3H, CH₃—CH₂—), 2.71 (q, J=7.58 Hz, 2H, CH₃—CH₂—), 2.98 (tq, J=7.09 and 3.67 Hz, 1H, —CH-(cypyl)), 7.57 (s, 1H, H-5), 7.86 (brs 1H, —NH—).

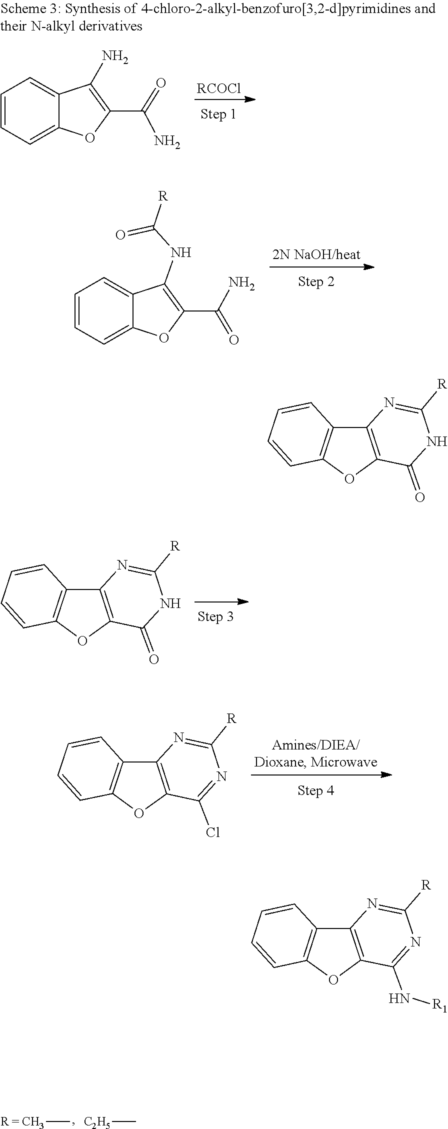

Scheme 3 shows a general method for the preparation of the benzofuropyrimidine intermediates from 3-aminobenzofuran-2-carboxamide, appropriate acid chloride or acid anhydride aqueous sodium hydroxide. This procedure afforded the corresponding benzofuropyrimidin-4-one which was converted to the corresponding 4-chloro-benzofuropyrimidines by the reaction of phosphorus oxychloride under refluxing conditions. The 4-aminoalkyl derivatives were synthesized by the reactions of the appropriate 4-chloro-benzofuropyrimidines with alkyl amines in the presence of a tert-amine and the microwave heating methodology.

### Step 1. 3-(Propanoylamino)benzofuran-2-carboxamide

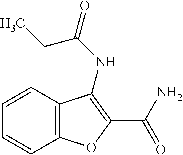 [SMILES: CCC(=O)Nc1c(C(N)=O)oc2ccccc12]

A suspension of 3-aminobenzofuran-2-carboxamide (363.0 mg, 2.06 mmol) in propionyl chloride (2.0 mL, 23.0 mmol) was heated at 45° C. overnight to give a colorless suspension. The solvent was evaporated in vacuo to afford a colorless solid. The solid was dissolved in ethyl acetate (50 mL) and washed with a saturated aqueous NaHCO₃ solution (1×25 mL), and brine (1×20 mL). The organic layer was removed, dried over anhydrous Na₂SO₄, filtered and evaporated in vacuo to give a colorless solid (517.0 mg, yield 100%). LC-MS analysis of the solid shows the desired with a purity >95% and the desired product's mass: m/z 233 (M+H) and m/z 255 (M+Na); Calcd for C₁₂H₁₂N₂O₃: 232.24.

### Step 2. 2-Ethyl-3H-benzofuro[3,2-d]pyrimidin-4-one

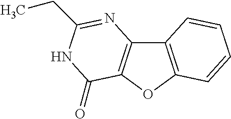 [SMILES: CCc1nc2c(oc3ccccc32)c(=O)[nH]1]

A suspension of 3-(propanoylamino)benzofuran-2-carboxamide (389.0 mg, 1.68 mmol) from step 1 in 2N NaOH solution (6.0 mL, 12.00 mol) was heated in a sand bath until a clear solution was obtained. The heating was stopped after 30 min and the reaction mixture was neutralized with 1 N HCl to give a colorless precipitate. The solid was filtered, washed with water and dried in vacuo to afford a colorless solid (340.5 mg, yield 95%). LC-MS analysis of the solid shows the desired product with a purity >98% and the desired product's mass: m/z 215 (M+H), m/z 237 (M+Na) and m/z 451 (2M+Na); Calcd for C₁₂H₁₀N₂O₂: 214.22.

### Step 3. 4-Chloro-2-ethyl-benzofuro[3,2-d]pyrimidine

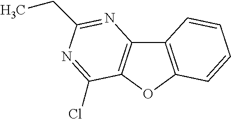 [SMILES: CCc1nc(Cl)c2oc3ccccc3c2n1]

A suspension of 2-ethyl-3H-benzofuro[3,2-d]pyrimidin-4-one (330.5 mg, 1.54 mmol) in phosphorus oxychloride (2 mL, 21.5 mmol) was heated at refluxing conditions for 2.5 h to give an orange-red solution. The reaction mixture was cooled to room temperature and evaporated in vacuo to give an orange viscous residue. The liquid was poured onto crushed ice-water to give a yellow-orange precipitate. The mixture was neutralized with a saturated NaHCO₃ solution and the precipitate was extracted with ethyl acetate (2×25 mL). The aqueous and the organic layers were separated. The organic layer was washed with brine (1×25 mL) and dried over anhydrous Na₂SO₄, filtered and evaporated in vacuo to afford a yellow-orange solid. The crude product was purified by silica-gel flash chromatography using 0 to 30% EtOAc in hexanes to afford a colorless crystalline solid (337.0 mg, yield 94%). LC-MS analysis of the solid showed the desired product with a purity >99% and the desired product's mass: m/z 233 (^{35Cl}M+H), and m/z 235 (^{37Cl}M+H); Calcd for C₁₂H₉ClN₂O: 214.22 ¹H NMR (400 MHz, DMSO-d₆): δ 1.37 (t, J=7.58 Hz, 3H, CH₃CH₂—), 3.05 (q, J=7.58 Hz, 2H, CH₃CH₂—), 7.59 (ddd, J=7.89, 7.15, 0.86 Hz, 1H), 7.82-7.88 (m, 1H), 7.91-7.99 (m, 1H), 8.19-8.29 (m, 1H); ¹H NMR spectrum of the product was consistent with the suggested structure of the product.

### 2-Ethyl-N-(3-phenylpropyl)benzofuro[3,2-d]pyrimidin-4-amine (1300)

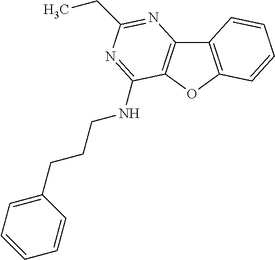 [SMILES: CCc1nc(NCCCc2ccccc2)c2oc3ccccc3c2n1]

A solution of 4-chloro-2-ethylbenzofuro[3,2-d]pyrimidine (71.8 mg, 0.31 mmol) from step 3, DIEA (85 μL, 0.5 mmol) and 3-phenylpropan-1-amine (140 μL, 0.985 mmol) in 1,4-dioxane (2.0 mL) was heated at 140° C. in a microwave reactor for 3 h to give a pale yellow suspension. The solvent was evaporated in vacuo and the crude residue was purified by reverse-phase preparative HPLC on a purified on a CombiFlashRf and a RediSep C18 (15.5 g gold) column and a gradient 10-60% acetonitrile in water containing 0.05% TFA. The pure fractions were evaporated in vacuo to afford a colorless glassy solid. The purified solid was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless crystalline solid (96.5 mg, yield 94%). LC-MS analysis of the freebase solid showed the desired product with a purity >98% and the desired product's mass: m/z 332 (M+H); Calcd for C₂₁H₂₁N₃O: 331.42. ¹H NMR (400 MHz, CDCl₃): δ 1.41 (t, J=7.58 Hz, 3H, CH₃—CH₂—), 2.09 (quin, J=7.34 Hz, 2H, —CH₂—CH₂—CH₂—), 2.80 (t, J=7.58 Hz, 2H, —CH₂—CH₂—CH₂—), 2.95 (q, J=7.58 Hz, 2H, —CH₂—CH₂—CH₂—), 3.77 (q, J=6.60 Hz, 2H, CH₃—CH₂—), 5.18 (brs/appt, 1H, —NH—), 7.16-7.35 (m, 5H, Ph-), 7.37-7.45 (m, 1H, Ph-), 7.53-7.61 (m, 2H, Ph-), 8.18 (d, J=7.82 Hz, 1H, Ph-).

### N-Butyl-2-ethylbenzofuro[3,2-d]pyrimidin-4-amine (1301)

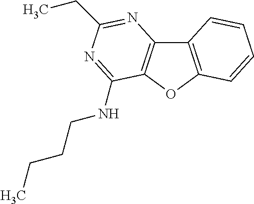 [SMILES: CCCCNc1nc(CC)nc2c1oc1ccccc12]

A solution of 4-chloro-2-ethyl-benzofuro[3,2-d]pyrimidine (70.3 mg, 0.30 mmol), DIEA (85 μL, 0.50 mmol) and n-butylamine (95 μL, 0.96 mmol) in 1,4-dioxane (2.0 mL) was heated at 140° C. in a microwave reactor for 3 h to give a pale yellow suspension. The solvent was evaporated in vacuo to afford a cream crystalline solid. The crude residue was purified by reverse-phase preparative HPLC on a CombiFlashRf and a RediSep C18 (15.5 g gold) column and a gradient 10-50% acetonitrile in water containing 0.05% TFA. The pure fractions were mixed together and evaporated in vacuo to afford a colorless viscous liquid containing a colorless crystalline solid. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless crystalline solid (75.4 mg, yield 93%). LC-MS analysis of the solid showed the desired product with a purity >98% and the desired product's mass: m/z 270 (M+H); Calcd for C₁₆H₁₉N₃O: 269.35. NMR (400 MHz, CDCl₃): δ 1.01 (t, J=7.34 Hz, 3H, CH₃—CH₂—CH₂—), 1.41 (t, J=7.58 Hz, 3H, CH₃—CH₂—), 1.50 (dq, J=14.89 Hz and 7.43 Hz, 2H, CH₃—CH₂—CH₂—), 1.72 (quip, J=7.33 Hz, 2H, CH₃—CH₃—CH₃—CH₂—NH—), 2.95 (q, J=7.66 Hz, 2H, CH₃—CH₂—CH₂—CH₂—), 3.73 (q, J=6.85 Hz, 2H, CH₃—CH₂—), 5.15 (brs/appt, 1H, —NH—), 7.37-7.44 (m, 1H, Ph-), 7.56 (d, J=3.91 Hz, 2H, Ph-), 8.18 (d, J=7.82 Hz, 1H, Ph-).

### N-cyclopropyl-2-ethyl-benzofuro[3,2-d]pyrimidin-4-amine (1142)

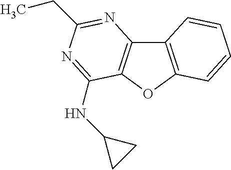 [SMILES: CCc1nc(NC2CC2)c2oc3ccccc3c2n1]

A solution of 4-chloro-2-ethyl-benzofuro[3,2-d]pyrimidine (72.3 mg, 0.31 mmol), DIEA (75 μL, 0.44 mmol) and cyclopropylamine (70 μL, 1.0 mmol) in 1,4-dioxane (1.5 mL) was heated at 140° C. in a microwave reactor for 4 h to give a pale yellow suspension. The solvent was evaporated in vacuo to afford a yellow-orange crystalline solid. The crude residue was purified by reverse-phase preparative HPLC on a Biotage KP-C18-HS (120 g) column and a gradient 10-60% acetonitrile in water containing 0.05% TFA. The pure fractions were mixed together and evaporated in vacuo to afford a colorless to pale yellow viscous residue. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless to cream solid (67.5 mg, yield 86%). LC-MS analysis of the solid showed the desired product with a purity >98% and the desired product's mass: m/z 254 (M+H); Calcd for C₁₅H₁₅N₃O: 253.31. ¹H NMR (400 MHz, DMSO-d₆): δ 0.61-0.68 (m, 2H, —CH₂—), 0.74-0.82 (m, 2H, —CH₂—), 1.31 (t, J=7.58 Hz, 3H, CH₃CH₂—), 2.81 (q, J=7.58 Hz, 2H, CH₃CH₂—), 3.08 (td, J=7.09, 3.67 Hz, 1H, —CH— (cypyl)), 7.45 (t, J=7.46 Hz, 1H), 7.61-7.68 (m, 1H), 7.69-7.75 (m, 1H), 8.04 (brs, 1H, —NH—), 8.04-8.08 (m, 1H).

### Step 1. 3-Acetamidobenzofuran-2-carboxamide

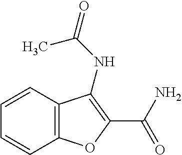 [SMILES: CC(=O)Nc1c(C(N)=O)oc2ccccc12]

A suspension of 3-aminobenzofuran-2-carboxamide (363.0 mg, 2.06 mmol) in acetic anhydride (5.0 mL, 52.9 mmol) was heated at 60° C. for 30 min to give a colorless solution. The reaction mixture was heated for another 30 min at 70° C. The solvent was evaporated in vacuo to afford a colorless crystalline solid. The solid was dissolved in ethyl acetate (25 mL) and washed with a saturated aqueous NaHCO₃ solution (1×10 mL), and brine (1×10 mL). The organic layer was separated, dried over anhydrous Na₂SO₄, filtered and evaporated in vacuo to give a colorless solid (466.0 mg, yield 100%). LC-MS analysis of the solid shows the desired product with a purity >95% and the desired product's mass: m/z 202 (M+H-NH₃), m/z 219 (M+H), and m/z 241 (M+Na); Calcd for C₁₁H₁₀N₂O₃: 218.21. The product will be used as such for the next step.

### Step 2. 2-Methyl-3H-benzofuro[3,2-d]pyrimidin-4-one

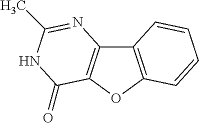

A suspension of 3-acetamidobenzofuran-2-carboxamide (466.0 mg, 2.14 mmol) in 2 N NaOH solution (20.0 mL, 40 mmol) was heated in a sand bath until a clear solution was obtained and the reaction mixture was stirred at 50° C. for 30 min. The heating was stopped and the reaction mixture was neutralized with 1 N HCl to give a colorless precipitate. The reaction mixture was stirred at room temperature for 10 min and the solid was filtered, washed with water (3×20 mL) and dried in vacuo to afford a colorless solid (390.0 mg, yield 91%). LC-MS analysis of the solid showed the desired product with a purity >98% and the desired product's mass: m/z 201 (M+H), m/z 223 (M+Na), and m/z 423 (2M+Na); Calcd for C₁₁H₈N₂O₂: 200.20.

### Step 3. 4-Chloro-2-methyl-benzofuro[3,2-d]pyrimidine

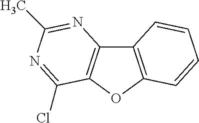

A suspension of 2-methyl-3H-benzofuro[3,2-cl]pyrimidin-4-one (390.0 mg, 1.95 mmol) in phosphorus oxychloride (5.0 mL, 53.64 mmol) was heated at refluxing conditions for 2 h. to give a thick cream-orange precipitate. Anhydrous DMF (0.5 mL) was added to the give an orange solution within 5 min. The reaction mixture was cooled to room temperature and evaporated in vacuo, to remove excess POCl₃ and the red liquid was poured onto crushed ice-water to give a dirty orange precipitate. The mixture was neutralized with a saturated NaHCO₃ solution and the precipitate was extracted with ethyl acetate (2×50 mL), the aqueous and the organic layers were separated, the organic layer was washed with brine (1×25 mL) and dried over anhydrous Na₂SO₄, filtered and evaporated in vacuo to afford a dirty yellow-orange solid. The crude product was purified by silica-gel flash chromatography using EtOAc in hexanes to afford a colorless crystalline solid (402.5 mg, yield 95%). LC-MS analysis of the solid showed the desired product with a purity >99% and the desired product's mass: m/z 219 (^{35Cl}M+H), and m/z 221 (^{37Cl}M+H); Calcd for C₁₁H₇ClN₂O: 218.64. ¹H NMR (400 MHz, DMSO-d₆): δ 2.77 (s, 3H, CH₃—), 7.54-7.63 (m, 1H), 7.81-7.89 (m, 1H), 7.92-7.99 (m, 1H), 8.24 (dd, J=7.82 and 0.73 Hz, 1H).

### 2-Methyl-N-(3-phenylpropyl)benzofuro[3,2-d]pyrimidin-4-amine (1299)

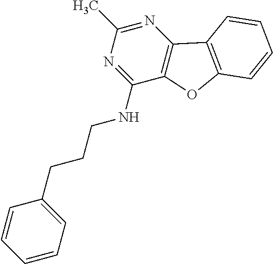 [SMILES: Cc1nc(NCCCc2ccccc2)c2oc3ccccc3c2n1]

A solution of 4-chloro-2-methyl-benzofuro[3,2-d]pyrimidine (70.6 mg, 0.32 mmol), DIEA (85 μL, 0.5 mmol) and 3-phenylpropan-1-amine (145 μL, 1.02 mmol) in 1,4-dioxane (2.0 mL) was heated at 140° C. in a microwave reactor for 3 h to give a colorless suspension. The solvent was evaporated in vacuo to afford a colorless to cream crystalline solid. The crude residue was purified by reverse-phase preparative HPLC on a purified on a CombiFlashRf and a RediSep C18 (15.5 g gold) column and a gradient 10-50% acetonitrile in water containing 0.05% TFA. The pure fractions were combined and evaporated in vacuo to afford a colorless viscous liquid. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a colorless solid (96.3 mg, yield 94%). LC-MS analysis of the freebase residue showed the desired product with a purity >98% and the desired product's mass: m/z 318 (M+H); Calcd for C₂₀H₁₉N₃O: 317.39. ¹H NMR (400 MHz, CDCl₃): δ 2.08 (quin, J=7.27 Hz, 2H, —CH₂—), 2.70 (s, 3H, 2-CH₃—), 2.80 (t, J=7.70 Hz, 2H, —CH₂—), 3.76 (q, J=6.60 Hz, 2H, —CH₂—), 5.17 (appt/brs, 1H, —NH—CH₂—), 7.18-7.35 (m, 5H, Ph-), 7.38-7.45 (m, 1H, Ph-), 7.53-7.61 (m, 2H, Ph-), 8.17 (d, J=8.07 Hz, 1H).

### N-Cyclopropyl-2-methyl-benzofuro[3,2-d]pyrimidin-4-amine (1151)

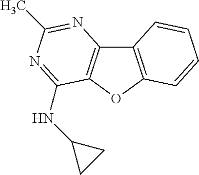 [SMILES: Cc1nc(NC2CC2)c2oc3ccccc3c2n1]

A solution of 4-chloro-2-methyl-benzofuro[3,2-d]pyrimidine (70.0 mg, 0.32 mmol), DIEA (80.0 μL, 0.467 mmol) and cyclopropylamine (70 μL, 1.0 mol) in 1,4-dioxane (2.0 mL) was heated at 140° C. in a microwave reactor for 4 h to give a pale yellow suspension. The solvent was evaporated in vacuo to afford an orange glassy residue. The crude residue was purified by reverse-phase preparative HPLC on a Biotage KP-C18-HS (120 g) column and a gradient 10-50% acetonitrile in water containing 0.05% TFA. The pure fractions were combined and evaporated in vacuo to afford a colorless to pale yellow viscous residue. The purified residue was dissolved in acetonitrile containing a trace of methanol and the solution was passed through a SiliaPrep Carbonate (Si—CO₃) 6 mL-1 g cartridge to neutralize TFA. The filtrate was evaporated in vacuo to afford a cream gummy solid (98.0 mg). The residue was dissolved in water containing a trace of acetonitrile and the solution was lyophilized to afford a cream lyophilized powder (85.7 mg, yield 100%). LC-MS analysis of the solid showed the desired product with a purity >98% and the desired product's mass: m/z 240 (M+H); Calcd for C₁₄H₁₃N₃O: 239.28. ¹H NMR (400 MHz, DMSO-d₆): δ 0.64-0.71 (m, 2H, —CH₂—), 0.75-0.85 (m, 2H, —CH₂—), 2.57 (s, 3H, 2-CH₃—), 3.11 (td, J=7.23 and 3.82 Hz, 1H, —CH— (cypyl)), 7.45-7.51 (m, 1H), 7.64-7.71 (m, 1H), 7.71-7.78 (m, 1H), 8.03-8.12 (m/d, 1H), 8.35 (brs, 1H, —NH—).

### N-butyl-2-methylbenzofuro[3,2-d]pyrimidin-4-amine (927)

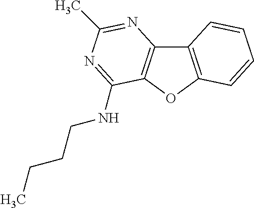 [SMILES: CCCCNc1nc(C)nc2c1oc1ccccc12]

A solution of 4-chloro-2-methyl-benzofuro[3,2-d]pyrimidine (70.0 mg, 0.32 mmol), DIEA (85 μL, 0.50 mmol) and n-butylamine (100 μL, 1.01 mmol) in 1,4-dioxane (2.0 mL) was heated at 140° C. in a microwave reactor for 3 h to give a pale yellow suspension. The crude residue was purified by reverse-phase preparative HPLC. The purified residue was dissolved in acetonitrile containing a trace of methanol and the solution was passed through a SiliaPrep Carbonate (Si—CO₃) 6 mL-1 g cartridge. The filtrate was evaporated in vacuo to afford a colorless crystalline solid (79.6 mg, yield 97%). LC-MS analysis of the freebase residue showed the desired product with a purity >98% and the desired product's mass: m/z 256 (M+H); Calcd for C₁₅H₁₇N₃O=255.32. ¹H NMR (400 MHz, CDCl₃): δ 1.01 (t, J=7.34 Hz, 3H, CH₃—CH₂—CH₂—), 1.50 (sxt, J=7.38 Hz, 2H, —CH₂—CH₂—CH₂—), 1.71 (quin, J=7.34 Hz, 2H, —CH₂—CH₂—CH₂—), 2.70 (s, 3H, 2-CH₃—), 3.72 (q, J=6.77 Hz, 2H, —CH₂—CH₂—CH₂—), 5.14 (brs/appt, 1H, —NH—), 7.41 (dt, J=7.95 and 4.10 Hz, 1H, Ph-), 7.57 (d, J=4.16 Hz, 2H, Ph-), 8.16 (d, J=7.82 Hz, 1H, Ph-).

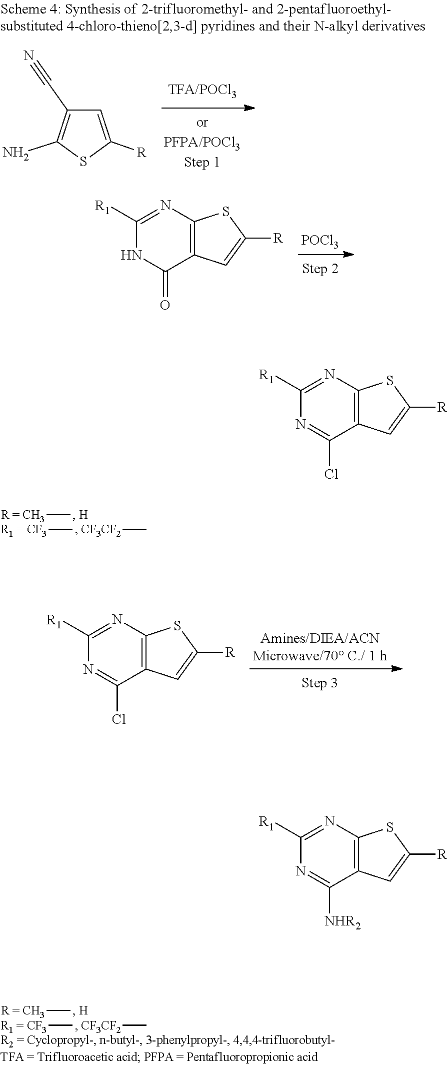

Scheme 4 shows a general method for the preparation of the fluorinated thienopyrimidine intermediates from 2-aminothiophene-3-carbontrile, appropriate fluorocarboxylic acid and POCl₃ in toluene. This one-pot procedure afforded the corresponding fluorinated thienopyrimidinone intermediates which were converted to the corresponding 4-chloro-thienopyrimidines by the reaction of phosphorus oxychloride under refluxing conditions.

The 4-aminoalkyl derivatives were synthesized by the reactions of the appropriate 4-chloro-thienopyrimidines with alkyl amines in the presence of a tert-amine and the microwave heating methodology.

### 6-Methyl-N-(3-phenylpropyl)-2-(trifluoromethyl)thieno[2,3-d]pyrimidin-4-amine (1536)

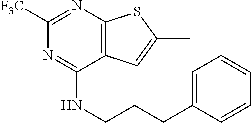 [SMILES: Cc1cc2c(NCCCc3ccccc3)nc(C(F)(F)F)nc2s1]

### Step 1. Preparation of 6-methyl-2-(trifluoromethyl)-3H-thieno[2,3-d]pyrimidin-4-one

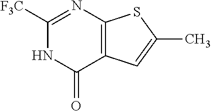

To a solution of 2-amino-5-methylthiophene-3-carbonitrile (2.79 g, 20.2 mmol) in trifluoroacetic acid (56.0 mL, 738 mmol) was added phosphorus oxychloride (4.0 mL, 43.3 mmol) and the reaction mixture was heated under refluxing conditions for 4 h and then cooled to room temperature. The trifluoroacetic acid was evaporated in vacuo to afford an orange viscous liquid. The residue was dissolved in water and neutralized with potassium carbonate till no effervescence of carbon dioxide occurred. The resulting yellow orange precipitate was filtered, washed with water (3×25 mL) and dried in vacuo to afford the desired product as a yellow solid (3.58 g; 76% yield). LC-MS analysis of solid showed the desired product with a purity >95% and the desired product's mass: m/z 236 (M+H); Calcd for C₈H₅F₃N₂OS: 234.20.

### Step 2. Preparation of 4-chloro-6-methyl-2-(trifluoromethyl)thieno[2,3-d]pyrimidine

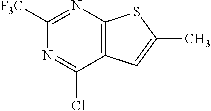

A suspension of 6-methyl-2-(trifluoromethyl)-3H-thieno[2,3-d]pyrimidin-4-one (3.58 g, 15.3 mmol) in phosphorus oxychloride (20.0 mL, 215 mmol) was heated at refluxing conditions overnight. The reaction mixture was cooled to room temperature and evaporated in vacuo, to remove excess POCl₃ to give an orange-brown viscous residue. The liquid was poured onto crushed ice-water and the mixture was neutralized with a saturated NaHCO₃ solution. The mixture was extracted with dichloromethane (2×25 mL), the aqueous and the organic layers were separated, the organic layer was evaporated in vacuo to afford an orange-brown gummy/crystalline residue. The crude product was dissolved in hot hexanes (100 mL) and filtered to remove suspended insoluble residue. The filtrate was evaporated in vacuo to afford an orange viscous liquid which solidified slowly to an orange crystalline solid (2.85 g). The solid was purified by silica-gel flash chromatography using 0 to 30% ethyl acetate in hexanes as eluent to afford the desired product as a very pale viscous liquid which solidified to a pale yellow crystalline solid (2.16 g, yield 56%). LC-MS analysis of the solid showed the desired product with a purity >98% and the desired product's mass: m/z 253 (^{35Cl}M+H), and m/z 255 (^{37Cl}M+H); Calcd for C₈H₄ClF₃N₂S: 252.64; ¹H NMR (400 MHz, CDCl₃): δ 2.73 (s, 3H), 7.22 (d, J=1.22 Hz, 1H, H-5); ¹⁹F NMR (376 MHz, CDCl₃): δ −69.02 (s) (2-CF₃—).

### Step 3. Preparation of 6-Methyl-N-(3-phenylpropyl)-2-(trifluoromethyl)thieno[2,3-d]pyrimidin-4-amine (1536)

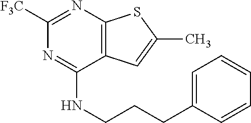 [SMILES: Cc1cc2c(NCCCc3ccccc3)nc(C(F)(F)F)nc2s1]

A solution of 4-chloro-6-methyl-2-(trifluoromethyl)thieno[2,3-d]pyrimidine (95.8 mg, 0.38 mmol), DIEA (140.0 μL, 0.80 mmol), and 3-phenylpropan-1-amine (115 μL, 0.81 mmol) in acetonitrile (4.00 mL) was heated at 70° C. in a microwave reactor for 1 h to give a very pale yellow solution. The solvent was evaporated in vacuo to afford a colorless crystalline residue. The crude residue was suspended in water (25 mL) and stirred at room temperature for 30 min. The precipitated solid was filtered, washed with water (3×25 mL) and dried in vacuo. The solid was dissolved in acetonitrile, filtered and evaporated in vacuo to afford a colorless to cream crystalline solid (134.0 mg, 100% yield). LC-MS analysis of the solid showed the desired product with a purity >96% and the desired product's mass: m/z 352 (M+H) and m/z 374 (M+Na); Calcd for C₁₇H₁₆F₃N₃S: 351.39. ¹H NMR (400 MHz, CDCl₃): δ 2.07 (quip, J=7.15 Hz, 2H, —CH₂—CH₂—CH₂—), 2.59 (d, J=0.98 Hz, 3H, 6-CH₃—), 2.78 (t, J=7.34 Hz, 2H, —CH₂—CH₂—CH₂—), 3.72 (q, J=7.00 Hz, 2H, —CH₂—CH₂—CH₂—), 5.11 (brs, 1H, —NH—), 6.63 (d, J=1.22 Hz, 1H, H-5), 7.18-7.26 (m, 3H, Ph-H), 7.29-7.36 (m, 2H, Ph-H). ¹⁹F NMR (376 MHz, CDCl₃): δ −70.09 (s) (2-CF₃—).

### N-(Phenylpropyl)-2-(trifluoromethyl)thieno[2,3-d]pyrimidin-4-amine (1537)

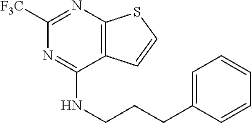 [SMILES: FC(F)(F)c1nc(NCCCc2ccccc2)c2ccsc2n1]

### Step 1. Preparation of 2-(trifluoromethyl)-3H-thieno[2,3-d]pyrimidin-4-one

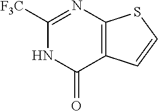 [SMILES: O=c1[nH]c(C(F)(F)F)nc2sccc12]

To a solution of 2-amino-5-methylthiophene-3-carbonitrile (1.24 g, 10.0 mmol) in trifluoroacetic acid (28.0 mL, 366 mmol) was added phosphorus oxychloride (2.0 mL, 21.5 mmol) and the reaction mixture was heated under refluxing conditions for 2 h and then cooled to room temperature. The trifluoroacetic acid was evaporated in vacuo to afford a blue-black crystalline solid. The residue was dissolved in water and neutralized with potassium carbonate till no effervescence of carbon dioxide occurred. The resulting dirty yellow green precipitate was filtered, washed with water (3×25 mL) and dried in vacuo to afford the desired product as a yellow-green solid (1.65 g; 75% yield). LC-MS analysis of solid showed the desired product's mass: m/z 221 (M+H); Calcd for C₇H₃F₃N₂OS:220.17.

### Step 2. Preparation of 4-chloro-2-(trifluoromethyl)thieno[2,3-d]pyrimidine

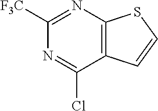 [SMILES: FC(F)(F)c1nc(Cl)c2ccsc2n1]

A suspension of 2-(trifluoromethyl)-3H-thieno[2,3-d]pyrimidin-4-one (1.65 g, 7.50 mmol) in phosphorus oxychloride (10.0 mL, 107.3 mmol) was heated at 75° C. for 6 h. The reaction mixture was cooled to room temperature and evaporated in vacuo, to remove excess POCl₃ to give a dark green viscous residue. The liquid was poured onto crushed ice-water and the mixture was neutralized with a saturated NaHCO₃ solution to give a brown-black precipitate. The solid was filtered and washed with water (2×25 mL) and dried in vacuo to afford a purple solid. The crude product was dissolved in hot hexanes (100 mL) and filtered to remove suspended insoluble residue. The filtrate was evaporated in vacuo to afford a yellow viscous liquid which solidified slowly to a yellow-green crystalline solid (0.68 g). The solid was purified by silica-gel flash chromatography using 0 to 30% ethyl acetate in hexanes as eluent to afford the desired product as a very pale viscous liquid which solidified to a pale yellow to cream crystalline solid (482.0 mg, yield 27%). LC-MS analysis of the solid showed the desired product with a purity >98% and the desired product's mass: m/z 239 (^{35Cl}M+H), and m/z 241 (^{37Cl}M+H); Calcd for C₇H₂ClF₃N₂S: 238.62. ¹H NMR (400 MHz, CDCl₃): δ 7.58 (d, J=6.00 Hz, 1H, H-5); 7.87 (d, J=6.11 Hz, 1H, H-6). ¹⁹F NMR (376 MHz, CDCl₃): δ −69.07 (s) (2-CF₃—).

### Step 3. N-(Phenylpropyl)-2-(trifluoromethyl)thieno[2,3-d]pyrimidin-4-amine (1537)

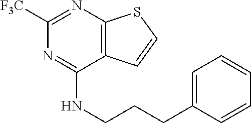 [SMILES: FC(F)(F)c1nc(NCCCc2ccccc2)c2ccsc2n1]

A solution of 4-chloro-2-(trifluoromethyl)thieno[2,3-d]pyrimidine (88.8 mg, 0.37 mmol), DIEA (130 μL, 0.76 mmol) and 3-phenylpropan-1-amine (110 μL, 0.77 mmol) in acetonitrile (4.00 mL) was heated at 70° C. in a microwave reactor for 1 h to give a very pale yellow solution. The solvent was evaporated in vacuo to afford a colorless crystalline residue and the crude residue was suspended in water (25 mL) and the suspension was stirred at room temperature for 30 min to give a colorless to cream solid. The solid was filtered, washed with water (3×25 mL) and dried in vacuo. The solid was dissolved in acetonitrile, filtered and evaporated in vacuo to afford a colorless to cream crystalline solid (125.0 mg, yield 99.6%). LC-MS analysis of the solid showed the desired product with a purity >96% and the desired product's mass: m/z 338 (M+H) and m/z 360 (M+Na); Calcd for C₁₆H₁₄F₃N₃S: 337.36. ¹H NMR (400 MHz, CDCl₃): δ 2.09 (quin, J=7.15 Hz, 2H, —CH₂—CH₂—CH₂—), 2.80 (t, J=7.34 Hz, 2H, —CH₂—CH₂—CH₂—), 3.75 (q, J=6.80 Hz, 2H, —CH₂—CH₂—CH₂—), 5.28 (brs, 1H, —NH—), 6.99 (d, J=6.11 Hz, 1H, H-5), 7.20-7.26 (m, 3H, Ph-H), 7.29-7.35 (m, 2H, Ph-H), 7.42 (d, J=6.11 Hz, 1H, H-6). ¹⁹F NMR (376 MHz, CDCl₃): δ −70.18 (s) (2-CF₃—).

### Step 1 and Step 2. 4-Chloro-6-methyl-2-(1,1,2,2,2-pentafluoroethyl)thieno[2,3-d]pyrimidine

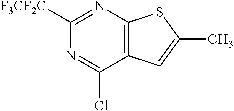

To a solution of a mixture of 2-amino-5-methylthiophene-3-carbonitrile (1.38 g, 10.0 mmol) and pentafluoropropionic acid (1.05 mL, 10.0 mmol) in toluene (10 mL) was added phosphorus oxychloride (3.0 mL, 32.2 mmol) to give a brown suspension. The reaction mixture was heated at 80° C. to give a dark green-brown solution with in 30 min and the reaction mixture was let heated at 80° C. overnight. The heating was discontinued and the solvent was evaporated in vacuo to give an orange-brown gummy residue. The gummy residue was poured onto crushed ice-water to give a dirty yellow precipitate. The mixture was neutralized with a saturated NaHCO₃ solution and the mixture was extracted with dichloromethane (3×25 mL), the aqueous and the organic layers were separated, the organic layer was dried over anhydrous Na₂SO₄, filtered and evaporated in vacuo to afford an orange-brown crystalline solid. The crude product was purified by silica-gel flash chromatography to afford a very pale viscous liquid which solidified to a pale yellow crystalline solid (1.00 g, yield 33%). LC-MS analysis of the solid showed the desired product with a purity >98% and the desired product's mass: m/z 303 (^{35Cl}M+H), and m/z 305 (^{37Cl}M+H); Calcd for C₉H₄ClF₅N₂S: 302.65. ¹H NMR (400 MHz, CDCl₃): δ 2.74 (d, J=1.22 Hz, 3H, 6-CH₃—), 7.22 (d, J=1.22 Hz, 1H, H-5); ¹⁹F NMR (376 MHz, CDCl₃): δ −82.22 (s, 3F, 2-CF₃CF₂—), −116.17 (s, 2F, CF₃CF₂—).

Silica-gel chromatography purification also afforded the intermediate product: 6-methyl-2-(perfluoroethyl)thieno[2,3-d]pyrimidin-4 (3H)-one as a yellow crystalline solid (650.0 mg). LC-MS analysis of the solid showed the intermediate product with a purity >95% and the intermediate product's mass: m/z 285 (M+H) and m/z 307 (M+Na); Calcd for C₉H₅F₅N₂OS: 284.21. LC-MS analysis of the bicarbonate washings showed the intermediate product: 6-methyl-2-(perfluoroethyl)thieno[2,3-d]pyrimidin-4(3H)-one only. The bicarbonate wash was neutralized with 6N HCl to afford a yellow precipitate. The precipitate was filtered, washed with water (2×25 mL) and dried in vacuo to afford a yellow solid. The solid was dissolved in acetonitrile, filtered and evaporated in vacuo to afford the intermediate product as a yellow crystalline solid (421.5 mg).

### Isolation of 6-methyl-2-(perfluoroethyl)thieno[2,3-d]pyrimidin-4(3H)-one

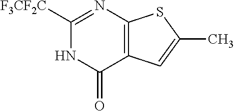

The combined intermediate product (1.045 g) from above was purified by silica-gel flash chromatography to afford a pale yellow crystalline solid (758.4 mg). LC-MS analysis of the solid showed the intermediate product with a purity >98% and the intermediate product's mass: m/z 285 (M+H) and m/z 307 (M+Na); Calcd for C₉H₅F₅N₂OS: 284.21 ¹H NMR (400 MHz, CDCl₃): δ 2.47 (d, J=0.98 Hz, 3H, 6-CH₃—), 6.75 (d, J=1.22 Hz, 1H, H-5), 9.01 (brs, 1H, —CO—NH—); ¹⁹F NMR (376 MHz, CDCl₃): δ −82.37 (s, 3F, 2-CF₃CF₂—), −122.03 (s, 2F, CF₃CF₂—).

### 6-Methyl-2-(1,1,2,2,2-pentafluoroethyl)-N-(3-phenylpropyl)thieno[2,3-d]pyrimidin-4-amine (1538)

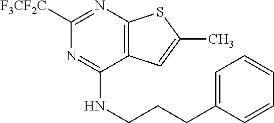

A solution of 4-chloro-6-methyl-2-(1,1,2,2,2-pentafluoroethyl)thieno[2,3-d]pyrimidine (89.2 mg, 0.29 5 mmol), DIEA (105 μL, 062 mmol) and 3-phenylpropan-1-amine (85 μL, 0.60 mmol) in acetonitrile (4.00 mL) was heated at 70° C. in a microwave reactor for 1 h and the solvent was evaporated in vacuo to afford a pale yellow to colorless crystalline residue. The crude residue was suspended in water (25 mL) and the suspension was stirred at room temperature for 30 min to give a colorless to cream solid. The solid was filtered, washed with water (3×25 mL) and dried in vacuo to afford a pale yellow to cream crystalline solid (114.0 mg, yield 96%). LC-MS analysis of the solid showed the desired product with a purity >98% and the desired product's mass: m/z 402 (M+H) and m/z 424 (M+Na); Calcd for C₁₈H₁₆F₅N₃S: 401.40. ¹H NMR (400 MHz, CDCl₃): δ 2.05 (quin, J=7.09 Hz, 2H, —CH₂—CH₂—CH₂—), 2.60 (d, J=0.98 Hz, 3H, 6-CH₃—), 2.76 (t, J=7.46 Hz, 2H, —CH₂—CH₂—CH₂—), 3.90 (q, J=6.85 Hz, 2H, —CH₂—CH₂—CH₂—), 5.13 (brs, 1H, —NH—), 6.64 (d, J=1.22 Hz, 1H, H-5), 7.18-7.26 (m, 3H, Ph-H), 7.28-7.34 (m, 2H, Ph-H); ¹⁹F NMR (376 MHz, CDCl₃): δ −82.05 (s, 3F, 2-CF₃CF₂—), −116.84 (s, 2F, CF₃CF₂—).

### Step 1 and Step 2. 4-Chloro-2-(1,1,2,2,2-pentafluoroethyl)thieno[2,3-d]pyrimidine

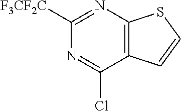

To a solution of a mixture of 2-aminothiophene-3-carbonitrile (1.39 g, 11.2 mmol) and pentafluoropropionic acid (1.20 mL, 11.42 mmol) in toluene (10 mL) was added phosphorus oxychloride (3.5 mL, 37.6 mmol) and the reaction mixture was heated at 80° C. overnight. LC-MS analysis of the reaction mixture after overnight heating showed an 1:1 mixture of the desired product and the intermediate product: 2-(perfluoroethyl)thieno[2,3-d]pyrimidin-4(3H)-one. A second batch of neat POCl₃ (5.0 mL) was added to the above residue and the reaction mixture was heated under refluxing conditions for 1 h to give a dark orange-brown suspension. The solvent was evaporated in vacuo to afford a dark orange-brown gummy solid. The gummy residue was poured onto crushed ice-water and the mixture was neutralized with a saturated NaHCO₃ solution and the mixture was extracted with dichloromethane (3×25 mL), the aqueous and the organic layers were separated, the organic layer was dried over anhydrous Na₂SO₄, filtered and evaporated in vacuo and the crude product was purified by silica-gel flash chromatography to afford a very pale viscous liquid which solidified to a very pale yellow to colorless crystalline solid (794.0 mg, yield 25%). LC-MS analysis of the solid showed the desired product with a purity >98% and the desired product's mass: m/z 289 (^{35Cl}M+H), and m/z 291 (^{37Cl}M+H); Calcd for C₈H₂ClF₅N₂S: 288.62 ¹H NMR (400 MHz, CDCl₃): δ 7.59 (d, J=6.00 Hz, 1H, H-5), 7.88 (d, J=6.11 Hz, 1H, H-6); ¹⁹F NMR (376 MHz, CDCl₃): δ −83.68 (s, 3F, 2-CF₃CF₂—), −116.13 (s, 2F, CF₃CF₂—).

### 2-(1,1,2,2,2-Pentafluoroethyl)-N-(3-phenylpropyl)thieno[2,3-d]pyrimidin-4-amine (1539)

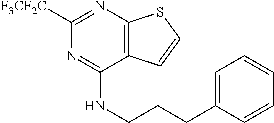

A solution of 4-chloro-2-(1,1,2,2,2-pentafluoroethyl)thieno[2,3-d]pyrimidine (88.3 mg, 0.306 mmol), DIEA (105 μL, 0.62 mmol) and 3-phenylpropan-1-amine (85 μL, 0.60 mmol) in acetonitrile (4.00 mL) was heated at 70° C. in a microwave reactor for 1 h and the solvent was evaporated in vacuo to afford a very pale yellow to colorless crystalline residue. The crude residue was suspended in water (25 mL) and the suspension was stirred at room temperature for 30 min to give a colorless to cream solid. The solid was filtered, washed with water (3×25 mL) and dried in vacuo to afford a colorless to cream crystalline solid (120 mg, 100% yield). LC-MS analysis of the solid showed the desired product with a purity >98% and the desired product's mass: m/z 388 (M+H) and m/z 410 (M+Na); Calcd for C₁₇H₁₄F₅N₃S: 387.37 ¹H NMR (400 MHz, CDCl₃): δ 2.08 (quin, J=7.15 Hz, 2H, —CH₂—CH₂—CH₂—), 2.78 (t, J=7.46 Hz, 2H, —CH₂—CH₂—CH₂—), 3.73 (q, J=6.90 Hz, 2H, —CH₂—CH₂—CH₂—), 5.30 (brs, 1H, —NH—), 7.00 (d, J=5.87 Hz, 1H, H-5), 7.19-7.26 (m, 3H, Ph-H), 7.28-7.35 (m, 2H, Ph-H), 7.43 (d, J=5.87 Hz, 1H, H-6). ¹⁹F NMR (376 MHz, CDCl₃): δ −82.01 (s, 3F, 2-CF₃CF₂—), −116.90 (s, 2F, CF₃CF₂—).

### N-Cyclopropyl-6-methyl-2-(trifluoromethyl)thieno[2,3-d]pyrimidin-4-amine (1540)

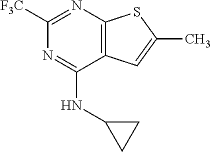 [SMILES: Cc1cc2c(NC3CC3)nc(C(F)(F)F)nc2s1]

A solution of 4-chloro-6-methyl-2-(trifluoromethyl)thieno[2,3-d]pyrimidine (91.0 mg, 0.36 mmol), DIEA (125 μL, 0.73 mmol) and cyclopropylamine (51 μL, 0.72 mmol) in acetonitrile (4.00 mL) was heated at 70° C. in a microwave reactor for 1 h and the solvent was evaporated in vacuo to afford a pale yellow viscous liquid. The crude residue was suspended in water (25 mL) and the suspension was stirred at room temperature for 30 min to give a colorless to cream solid. The solid was filtered, washed with water (3×25 mL) and dried in vacuo. The solid was dissolved in acetonitrile, filtered and evaporated in vacuo to afford a colorless to cream solid (93.0 mg, yield 98%). LC-MS analysis of the solid showed the desired product with a purity >96% and the desired product's mass: m/z 274 (M+H) and m/z 296 (M+Na); Calcd for C₁₁H₁₀F₃N₃S: 273.28. ¹H NMR (400 MHz, CDCl₃): δ 0.65-0.77 (m, 2H, —CH₂—), 0.93-1.03 (m, 2H, —CH₂—), 2.61 (d, J=1.22 Hz, 3H, 6-CH₃—), 3.04 (td, J=6.66 Hz, and 3.06 Hz, 1H, —CH-(cypyl)), 5.61 (brs, 1H, —NH—), 7.11 (brs, 1H, H-5). ¹⁹F NMR (376 MHz, CDCl₃): δ −70.06 (s) (2-CF₃—).

### N-Cyclopropyl-2-(trifluoromethyl)thieno[2,3-d]pyrimidin-4-amine (CWHM-0001541)

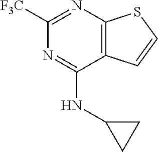 [SMILES: FC(F)(F)c1nc(NC2CC2)c2ccsc2n1]

A solution of 4-chloro-2-(trifluoromethyl)thieno[2,3-d]pyrimidine (94.2 mg, 0.395 mmol), DIEA (135 μL, 0.79 mmol) and cyclopropylamine (56 μL, 0.79 mmol) in acetonitrile (4.00 mL) was heated at 70° C. in a microwave reactor for 1 h to give a very pale yellow solution. LC-MS analysis of the reaction mixture after 1 h and the solvent was evaporated in vacuo to afford a pale yellow viscous liquid. The crude residue was suspended in water (25 mL) and the suspension was stirred at room temperature for 30 min to give a colorless to cream solid. The solid was filtered, washed with water (3×25 mL) and dried in vacuo. The solid was dissolved in acetonitrile, filtered and evaporated in vacuo to afford a colorless to cream solid (68.0 mg, 67% yield). LC-MS analysis of the solid showed the desired product with a purity >98% and the desired product's mass: m/z 260 (M+H); Calcd for C₁₀H₈F₃N₃S: 259.25. ¹H NMR (400 MHz, CDCl₃): δ 0.71-0.80 (m, 2H, —CH₂—), 0.94-1.08 (m, 2H, —CH₂—), 3.08 (td, J=6.66 Hz, and 3.18 Hz, 1H, —CH-(cypyl)), 5.79 (brs, 1H, —NH—), 7.46 (d, J=5.87 Hz, H-6), 7.52 (brd, J=7.07 Hz, 1H, H-5). ¹⁹F NMR (376 MHz, CDCl₃): δ −70.15 (s) (2-CF₃—).

### N-Cyclopropyl-6-methyl-2-(perfluoroethyl)thieno[2,3-d]pyrimidin-4-amine (1542)

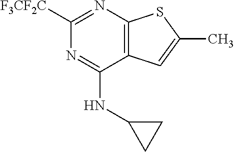

A solution of 4-chloro-6-methyl-2-(1,1,2,2,2-pentafluoroethyl)thieno[2,3-d]pyrimidine (101.3 mg, 0.335 mmol), DIEA (115 μL, 0.67 mmol) and cyclopropylamine (50 μL, 0.72 mmol) in acetonitrile (4.00 mL) was heated at 70° C. in a microwave reactor for 1 h and the solvent was evaporated in vacuo to afford a pale yellow viscous liquid. The crude residue was suspended in water (25 mL) and the suspension was stirred at room temperature for 30 min to give a cream to pale yellow solid. The crude product was purified by silica-gel flash chromatography to afford a very pale viscous liquid which solidified to a pale yellow to cream crystalline solid (98.8 mg, 91% yield). LC-MS analysis of the solid showed the desired product with a purity >98%. and the desired product's mass: m/z 402 (M+H) and m/z 424 (M+Na); Calcd for C₁₂H₁₀F₅N₃S: 323.29. ¹H NMR (400 MHz, CDCl₃): δ 0.62-0.78 (m, 2H, —CH₂—), 0.88-1.05 (m, 2H, —CH₂—), 2.61 (d, J=0.98 Hz, 3H, 6-CH₃—), 3.01 (td, J=6.60 Hz, and 3.18 Hz, 1H, —CH-(cypyl)), 5.59 (brs, 1H, —NH—), 7.07 (brs, 1H, H-5). ¹⁹F NMR (376 MHz, CDCl₃): δ −82.12 (s, 3F, 2-CF₃CF₂—), −116.99 (s, 2F, CF₃CF₂—).

### N-Cyclopropyl-2-(perfluoroethyl)thieno[2,3-d]pyrimidin-4-amine (1543)

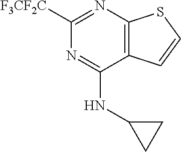

A solution of 4-chloro-2-(1,1,2,2,2-pentafluoroethyl)thieno[2,3-d]pyrimidine (91.0 mg, 0.315 mmol), DIEA (110 μL, 0.65 mmol) and cyclopropylamine (45 μL, 0.64 mmol) in acetonitrile (4.00 mL) was heated at 70° C. in microwave reactor for 1 h and the solvent was evaporated in vacuo to afford a pale yellow viscous liquid. The crude residue was suspended in water (25 mL) and the suspension was stirred at room temperature for 30 min and the solid was filtered, washed with water (3×25 mL) and dried in vacuo to give a cream gummy solid. The crude product was purified by silica-gel flash chromatography to afford a colorless viscous liquid which solidified a pale yellow to cream crystalline solid (79.7 mg, yield 82%). LC-MS analysis of the solid showed the desired product with a purity >98% and the desired product's mass: m/z 310 (M+H); Calcd for C₁₁H₈F₅N₃S: 309.26. ¹H NMR (400 MHz, CDCl₃): δ 0.75 (brs/m, 2H, —CH₂—), 0.92-1.05 (m, 2H, —CH₂—), 3.04 (td, J=6.68 Hz, and 3.67 Hz, 1H, —CH-(cypyl)), 5.77 (brs, 1H, —NH—), 7.46 (s, 1H, H-5), 7.47 (s, 1H, H-6). ¹⁹F NMR (376 MHz, CDCl₃): δ −82.09 (s, 3F, 2-CF₃CF₂—), −117.06 (s, 2F, CF₃CF₂—).

### N-Butyl-6-methyl-2-(trifluoromethyl)thieno[2,3-d]pyrimidin-4-amine (1544)

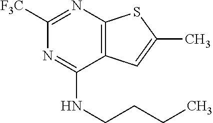 [SMILES: CCCCNc1nc(C(F)(F)F)nc2sc(C)cc12]

A solution of 4-chloro-6-methyl-2-(trifluoromethyl)thieno[2,3-d]pyrimidine (112.5 mg, 0.45 mmol), DIEA (155 μL, 0.91 mmol) and n-butylamine (90 μL, 0.92 mmol) in acetonitrile (4.00 mL) was heated at 70° C. in a microwave reactor for 1 h to give a very pale yellow solution and the solvent was evaporated in vacuo to afford a pale yellow crystalline/gummy solid. The crude residue was suspended in water (25 mL) and the suspension was stirred at room temperature for 30 min and the solid was filtered, washed with water (3×25 mL) and dried in vacuo to afford a colorless to cream solid (121.1 mg, yield 94%). LC-MS analysis of the solid showed the desired product with a purity >98% and the desired product's mass: m/z 290 (M+H); Calcd for C₁₂H₁₄F₃N₃S: 289.32. ¹H NMR (400 MHz, CDCl₃): δ 0.99 (t, J=7.34 Hz, 3H, —CH₂—CH₂—CH₂—CH₃), 1.45 (dq, J=14.95 Hz and 7.33 Hz, 2H, —CH₂—CH₂—CH₂—CH₃), 1.61-1.73 (m, 2H, —CH₂—CH₂—CH₂—CH₃), 2.61 (d, J=0.98 Hz, 3H, 6-CH₃—), 3.67 (td, J=7.09 Hz and 5.87 Hz, 2H, —NH—CH₂—CH₂—CH₂—CH₃), 5.19 (brs, 1H, —NH—), 6.83 (d, J=1.22 Hz, 1H, H-5). ¹⁹F NMR (376 MHz, CDCl₃): δ −70.13 (s) (2-CF₃—).

### N-Butyl-2-(trifluoromethyl)thieno[2,3-d]pyrimidin-4-amine (1545)

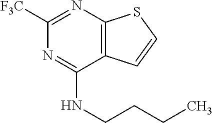 [SMILES: CCCCNc1nc(C(F)(F)F)nc2sccc12]

A solution of 4-chloro-2-(trifluoromethyl)thieno[2,3-d]pyrimidine (106.5 mg, 0.45 mmol), DIEA (130 μL, 0.76 mmol) and n-butylamine (90 μL, 0.92 mmol) in acetonitrile (4.00 mL) was heated at 70° C. in a microwave reactor for 1 h to give a very pale yellow solution and the solvent was evaporated in vacuo to afford a pale blue-green gummy/crystalline residue. The crude residue was suspended in water (25 mL) and the suspension was stirred at room temperature for 30 min to give a pale blue-yellow oily residue. The mixture was diluted with ethyl acetate (25 mL) and the aqueous and the organic layers were separated. The organic layer was washed with water (2×25 mL), and evaporated in vacuo to afford a pale yellow gummy solid. The solid was dissolved in acetonitrile, filtered and evaporated in vacuo to afford pale yellow to cream solid (116.0 mg, yield 95%). LC-MS analysis of the solid showed the desired product with a purity >98% and the desired product's mass: m/z 276 (M+H); Calcd for C₁₁H₁₂F₃N₃S: 275.29. ¹H NMR (400 MHz, CDCl₃): δ 1.00 (t, J=7.34 Hz, 3H, —CH₂—CH₂—CH₂—CH₃), 1.46 (dq, J=15.04 Hz and 7.38 Hz, 2H, —CH₂—CH₂—CH₂—CH₃), 1.64-1.76 (m, 2H, —CH₂—CH₂—CH₂—CH₃), 3.70 (td, J=7.09 Hz and 5.87 Hz, 2H, —NH—CH₂—CH₂—CH₂—CH₃), 5.38 (brs, 1H, —NH—), 7.20 (d, J=5.87 Hz, 1H, H-5), 7.46 (d, J=6.11 HZ, 1H, H-6). ¹⁹F NMR (376 MHz, CDCl₃): δ −70.22 (s) (2-CF₃—).

### N-Butyl-6-methyl-2-(perfluoroethyl)thieno[2,3-d]pyrimidin-4-amine (1546)

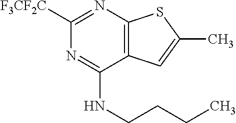

A solution of 4-chloro-6-methyl-2-(1,1,2,2,2-pentafluoroethyl)thieno[2,3-d]pyrimidine (109 mg, 0.36 mmol), DIEA (125 μL, 0.73 mmol), and n-butylamine (75 μL, 0.76 mmol) in acetonitrile (4.00 mL) was heated at 70° C. in a microwave reactor for 1 h to give a yellow-orange solution. LC-MS analysis of the reaction mixture after 1 h and the solvent was evaporated in vacuo to afford a pale yellow viscous liquid. The crude residue was suspended in water (25 mL) and the suspension was stirred at room temperature for 30 min to give a cream to pale yellow solid. The solid was filtered, washed with water (3×25 mL) and dried in vacuo to afford a pale yellow to cream solid (112.1 mg, yield 92%). LC-MS analysis of the solid showed the desired product with a purity >98% and the desired product's mass: m/z 340 (M+H); Calcd for C₁₃H₁₄F₅N₃S: 339.33. ¹H NMR (400 MHz, CDCl₃): δ 0.98 (t, J=7.46 Hz, 3H, —CH₂—CH₂—CH₂—CH₃), 1.44 (dq, J=14.98 Hz and 7.40 Hz, 2H, —CH₂—CH₂—CH₂—CH₃), 1.62-1.71 (m, 2H, —CH₂—CH₂—CH₂—CH₃), 2.61 (d, J=0.98 Hz, 3H, 6-CH₃—), 3.65 (td, J=7.09 Hz and 5.87 Hz, 2H, —NH—CH₂—CH₂—CH₂—CH₃), 5.21 (brs, 1H, —NH—), 6.83 (d, J=1.22 Hz, 1H, H-5). ¹⁹F NMR (376 MHz, CDCl₃): δ −82.09 (s, 3F, 2-CF₃CF₂—), −116.89 (s, 2F, CF₃CF₂—).

### N-Butyl-2-(perfluoroethyl)thieno[2,3-d]pyrimidin-4-amine (1547)

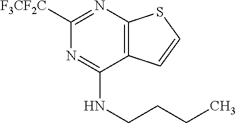

A solution of 4-chloro-2-(1,1,2,2,2-pentafluoroethyl)thieno[2,3-d]pyrimidine (114.7 mg, 0.40 mmol), DIEA (140 μL, 0.82 mmol) and n-butylamine (80 μL, 0.81 mmol) in acetonitrile (4.00 mL) was heated at 70° C. in a microwave reactor for 1 h to give a pale yellow-green solution and the solvent was evaporated in vacuo to afford a dirty yellow gummy residue. The crude residue was suspended in water (25 mL) and the suspension was stirred at room temperature for 30 min to give a colorless to very pale blue solid. The crude product was purified by silica-gel flash chromatography to afford a colorless viscous liquid which solidified to a colorless to cream crystalline solid (123.0 mg, yield 95%). LC-MS analysis of the solid showed the desired product with a purity >98% and the desired product's mass: m/z 326 (M+H); Calcd for C₁₂H₁₂F₅N₃S: 325.30. ¹H NMR (400 MHz, CDCl₃): δ 0.99 (t, J=7.34 Hz, 3H, —CH₂—CH₂—CH₂—CH₃), 1.45 (dq, J=14.89 Hz and 7.43 Hz, 2H, —CH₂—CH₂—CH₂—CH₃), 1.63-1.76 (m, 2H, —CH₂—CH₂—CH₂—CH₃), 3.68 (td, J=7.09 Hz and 5.87 Hz, 2H, —NH—CH₂—CH₂—CH₂—CH₃), 5.40 (brs, 1H, —NH—), 7.20 (d, J=5.87 Hz, 1H, H-5), 7.46 (d, J=5.87 Hz, 1H, H-6). ¹⁹F NMR (376 MHz, CDCl₃): δ −82.05 (s, 3F, 2-CF₃CF₂—), −116.95 (s, 2F, CF₃CF₂—).

### N-(4,4,4-Trifluorobutyl)-2-(trifluoromethyl)thieno[2,3-d]pyrimidin-4-amine (1548)

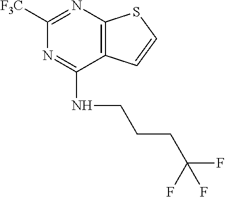 [SMILES: FC(F)(F)CCCNc1nc(C(F)(F)F)nc2sccc12]

A solution of 4-chloro-2-(trifluoromethyl)thieno[2,3-d]pyrimidine (78.6 mg, 0.33 mmol), DIEA (100 μL, 0.58 mmol) and 4,4,4-trifluorobutan-1-amine (75 μL, 0.65 mmoL) in acetonitrile (4.0 mL) was heated at 70° C. in a microwave reactor for 1 h to give a very pale yellow solution and the solvent was evaporated in vacuo to afford a pale yellow crystalline solid. The crude residue was suspended in water (25 mL) and the suspension was stirred at room temperature for 30 min to give a colorless to cream solid. The solid was filtered, washed with water (3×25 mL) and dried in vacuo to afford a pale yellow to cream solid (98.5 mg; yield 91%). LC-MS analysis of the solid showed the desired product with a purity >98% and the desired product's mass: m/z 330 (M+H); Calcd for C₁₁H₉F₆N₃S: 329.26. ¹H NMR (400 MHz, CDCl₃): δ 1.94-2.10 (m, 2H, —CH₂—CH₂—CH₂—CF₃), 2.14-2.34 (m, 2H, —CH₂—CH₂—CH₂—CF₃), 3.81 (q, J=6.68 Hz, 2H, —NH—CH₂—CH₂—CH₂—CF₃), 5.49 (appt/brs, 1H, —NH—), 7.21 (d, J=6.11 Hz, 1H, H-5), 7.51 (d, J=6.11 Hz, 1H, H-6). ¹⁹F NMR (376 MHz, CDCl₃): δ −66.09 (t, J=10.90 Hz, 3F, CF₃—CH₂—), −70.25 (s, 3F, 2-CF₃—).

### 2-(Perfluoroethyl)-N-(4,4,4-trifluorobutyl)thieno[2,3-d]pyrimidin-4-amine (1549)

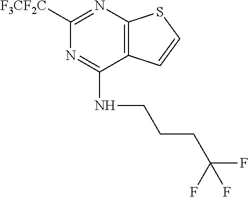

A solution of 4-chloro-2-(perfluoroethyl)thieno[2,3-d]pyrimidine (82.6 mg, 0.29 mmol), DIEA (90 μL, 0.53 mmol) and 4,4,4-trifluorobutan-1-amine (65 μL, 0.57 mmol) in acetonitrile (4.0 mL) was heated at 70° C. in a microwave reactor for 1 h to give a very pale yellow solution and the solvent was evaporated in vacuo to afford a very pale yellow gummy/crystalline solid The crude residue was partitioned between ethyl acetate (25 mL) and water (50 mL) and the mixture was stirred at room temperature for 30 min. The aqueous and organic layers were separated and the organic layer was washed with water (1×25 mL). The organic layer was evaporated in vacuo to afford a colorless gummy solid. The solid isolated from ethyl acetate layer was suspended in water (25 mL) and stirred at room temperature for 2 h to afford a colorless precipitate. The solid was filtered, washed with water (1×25 mL) and dried in vacuo to afford the desired product as a colorless to cream solid (105.0 mg; yield 97%). LC-MS analysis of the solid showed the desired product with a purity >98% and the desired product's mass: m/z 380 (M+H); Calcd for C₁₂H₉F₈N₃S: 379.27 ¹H NMR (400 MHz, CDCl₃): δ 1.93-2.07 (m, 2H, —CH₂—CH₂—CH₂—CF₃), 2.14-2.32 (m, 2H, —CH₂—CH₂—CH₂—CF₃), 3.78 (q, J=6.60 Hz, 2H, —NH—CH₂—CH₂—CH₂—CF₃), 5.51 (appt/brs, 1H, —NH—), 7.21 (d, J=5.87 Hz, 1H, H-5), 7.52 (d, J=5.87 Hz, 1H, H-6). ¹⁹F NMR (376 MHz, CDCl₃): δ −66.16 (t, J=10.90 Hz, 3F, CF₃—CH₂—), −82.10 (s, 3F, 2-CF₃CF₂—), −116.98 (s, 2F, CF₃CF₂—).

### 6-Methyl-N-(4,4,4-trifluorobutyl)-2-(trifluoromethyl)thieno[2,3-d]pyrimidin-4-amine (1551)

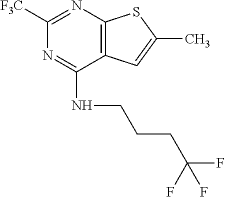 [SMILES: Cc1cc2c(NCCCC(F)(F)F)nc(C(F)(F)F)nc2s1]

A solution of 4-chloro-6-methyl-2-(trifluoromethyl)thieno[2,3-d]pyrimidine (92.4 mg, 0.37 mmol), DIEA (115 μL, 0.67 mmol) and 4,4,4-trifluorobutan-1-amine (80 μL, 0.70 mmol) in acetonitrile (4.0 mL) was heated at 70° C. in a microwave reactor for 1 h to give a very pale yellow solution and the solvent was evaporated in vacuo to afford a pale yellow to cream solid. The crude residue was suspended in water (25 mL) and the suspension was stirred at room temperature for 30 min to give a colorless to cream solid. The precipitated solid was filtered, washed with water (3×25 mL) and dried in vacuo to afford a colorless to cream solid (115.0 mg; yield 92%). LC-MS analysis of the solid showed the desired product with a purity >98% and the desired product's mass: m/z 344 (M+H); Calcd for C₁₂H_{H}F₆N₃S: 343.29. ¹H NMR (400 MHz, CDCl₃): δ 1.93-2.08 (m, 2H, —CH₂—CH₂—CH₂—CF₃), 2.12-2.35 (m, 2H, —CH₂—CH₂—CH₂—CF₃), 2.62 (d, J=0.98 Hz, 3H, 6-CH₃—), 3.77 (q, J=6.60 Hz, 2H, —NH—CH₂—CH₂—CH₂—CF₃), 5.29 (brs, 1H, —NH—), 6.84 (d, J=1.22 Hz, 1H, H-5). ¹⁹F NMR (376 MHz, CDCl₃): δ −66.09 (t, J=10.90 Hz, 3F, CF₃—CH₂—), −70.15 (s, 3F, 2-CF₃—).

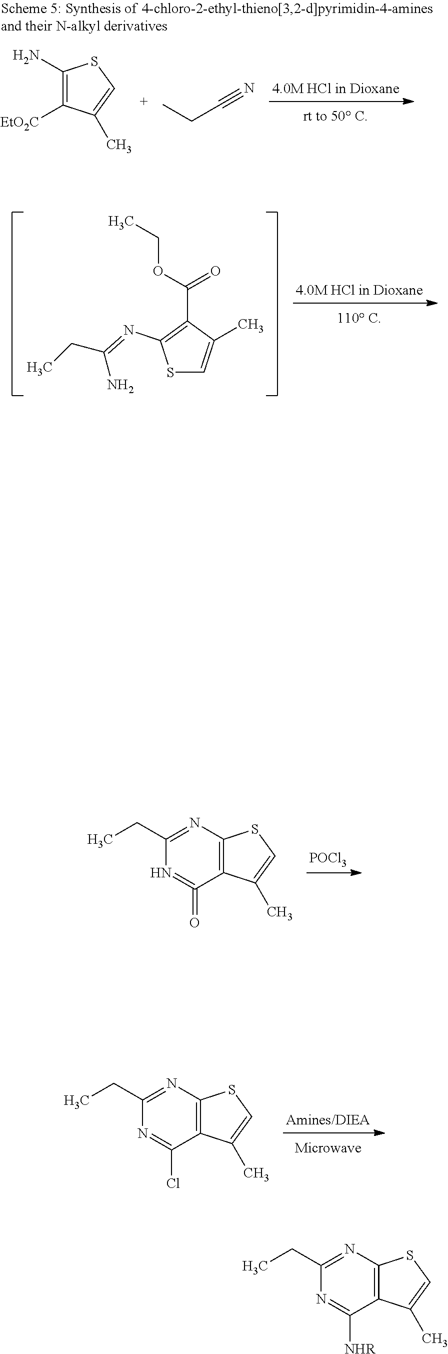

Scheme 5 shows a general method for the preparation of the 5-methyl-thienopyrimidine intermediates from 2-aminothiophene-3-carboxyesters, appropriate alkyl nitriles and dry HCl in 1,4-dioxane. This procedure afforded the corresponding thienopyrimidinones which were converted to the corresponding 4-chloro-thienopyrimidines by the reaction of phosphorus oxychloride under refluxing conditions.

The 4-aminoalkyl derivatives were synthesized by the reactions of the appropriate 4-chloro-thienopyrimidines with alkyl amines in the presence of a tert-amine and the microwave heating methodology.

### Step 1. 2-Ethyl-5-methylthieno[2,3-d]pyrimidin-4(3H)-one

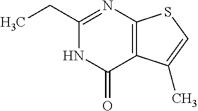

A dark red mixture of ethyl 2-amino-4-methylthiophene-3-carboxylate (793.0 mg, 4.28 mmol) and propionitrile (1.0 mL, 14.0 mmol) was treated with 4.0 M HCl in dioxane (3 mL; 12 mmol) at room temperature. The reaction mixture quickly turned to a burgundy red solution first and then to a thick pinkish cream paste. An additional 2 mL 4.0 M HCl/dioxane was added after 30 min and the reaction mixture was heated at 50° C. to give a pink-red solution. LC-MS analysis of the reaction mixture after 1.5 h showed the uncyclized intermediate product: (Z)-ethyl 2-((1-aminopropylidene)amino)-4-methylthiophene-3-carboxylate and the intermediate's mass: m/z 241 (M+H). After heating at 50° C. for 1.5 h, the reaction mixture was heated at 110° C. to give a dark red solution. A colorless microcrystalline solid begin to form within 1 h. Another 2 mL of 4.0 M HCl in dioxane was added and the mixture was heated at 110° C. overnight. The solvent was evaporated in vacuo to afford a burgundy-cream solid. The solid was dissolved in acetonitrile (20 mL) and cooled to room temperature to afford a cream crystalline precipitate. The solid was filtered, washed with acetonitrile (2×10 mL) and dried in vacuo to give a dirty cream crystalline solid (640.8 mg, yield 77%). LC-MS analysis of the solid showed the desired product with a purity >98% and the desired product's mass: m/z 195 (M+H), and m/z 217 (M+Na); Calcd for C₉H₁₀N₂OS=194.25. ¹H NMR (400 MHz, DMSO-d₆): δ 1.19 (t, J=7.46 Hz, 3H, CH₃CH₂—), 2.61 (q, J=7.46 Hz, 2H, CH₃CH₂—), 3.33 (s, 3H, CH₃—), 7.03 (d, J=0.98 Hz, 1H, H-6), 7.34 (brs, 1H, —CONH).

### Step 2. 4-Chloro-2-ethyl-5-methylthieno[2,3-d]pyrimidine

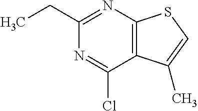

A suspension of 2-ethyl-5-methyl-3H-thieno[2,3-d]pyrimidin-4-one (596 mg, 3.07 mmol) in phosphorus oxychloride (4 mL, 42.9 mmol) was heated at refluxing conditions for 3 h. The solvent was evaporated in vacuo to afford a light orange-brown viscous liquid. The liquid was poured onto crushed ice-water to give a dirty yellow precipitate. The mixture was neutralized with a saturated NaHCO₃ and the precipitate was filtered, washed with water (2×25 mL) and dried in vacuo to afford a light yellow solid (538.0 mg; yield 83%). LC-MS analysis of the solid showed the desired product with a purity >98% and the desired product's mass: m/z 213 (^{35Cl}M+H), and m/z 215 (^{37Cl}M+H); Calcd for C₉H₉ClN₂S=212.70. ¹H NMR (400 MHz, CDCl₃): δ 1.42 (t, J=7.58 Hz, 3H, CH₃CH₂—), 2.67 (d, J=1.22 Hz, 3H, 5-CH₃—), 3.06 (q, J=7.58 Hz, 2H, CH₃CH₂—), 7.11 (d, J=1.22 Hz, 1H, H-6).

### 2-Ethyl-5-methyl-N-(3-phenylpropyl)thieno[2,3-d]pyrimidin-4-amine (1698)

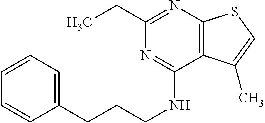 [SMILES: CCc1nc(NCCCc2ccccc2)c2c(C)csc2n1]

A solution of 4-chloro-2-ethyl-5-methylthieno[2,3-d]pyrimidine (62.5 mg, 0.294 mmol), DIEA (105 μL, 0.61 mmol) and 3-phenylpropan-1-amine (130 μL, 0.92 mmol) in 1,4-dioxane (2.00 mL) was heated at 140° C. in a microwave reactor for 3 h to give a yellow solution and the solvent was evaporated in vacuo to afford a yellow gummy residue. The gummy residue was partitioned between water (25 mL) and ethyl acetate (25 mL). The organic layer was removed, washed with brine (1×10 mL) and evaporated in vacuo to afford an orange viscous liquid. The crude residue was purified by silica-gel flash chromatography to afford a very pale yellow viscous liquid which solidified to a pale yellow to cream crystalline/waxy solid (85.5 mg, yield 94%). LC-MS analysis of the solid showed the desired product with a purity >98% and the desired product's mass: m/z 312 M+H). Calcd for C₁₈H₂N₃S=311.45. ¹H NMR (400 MHz, CDCl₃): δ 1.36 (t, J=7.58 Hz, 3H, CH₃—CH₂—), 2.05 (quin, J=7.27 Hz, 2H, —CH₂—CH₂—CH₂—), 2.50 (d, J=1.22 Hz, 3H, 5-CH₃—), 2.77 (t, J=7.46 Hz, 2H, —CH₂—CH₂—CH₂-Ph), 2.83 (q, J=7.58 Hz, 2H, CH₃—CH₂—), 3.68 (td, J=6.97 and 5.87 Hz, 2H, —NH—CH₂—CH₂—CH₂-Ph), 5.34 (brs, 1H, —NH—), 6.71 (d, J=1.22 Hz, 1H, H-6), 7.18-7.25 (m, 3H, Ph-H), 7.27-7.33 (m, 2H, Ph-H).

### N-Butyl-2-ethyl-5-methylthieno[2,3-d]pyrimidin-4-amine (1699)

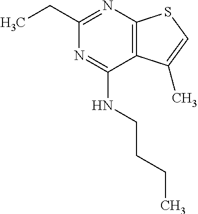 [SMILES: CCCCNc1nc(CC)nc2scc(C)c12]

A solution of 4-chloro-2-ethyl-5-methylthieno[2,3-d]pyrimidine (57.0 mg, 0.268 mmol), DIEA (100 μL, 0.58 mmol) and butylamine (60 μL, 0.61 mmol) in 1,4-dioxane was heated at 140° C. in a microwave reactor for 3 h and the solvent was evaporated in vacuo to give a yellow-orange viscous liquid. The liquid was partitioned between water (25 mL) and ethyl acetate (25 mL). The organic layer was removed, washed with brine (1×10 mL) and evaporated in vacuo to afford an orange viscous liquid. The crude residue was purified by silica-gel flash chromatography to afford a very pale yellow viscous liquid which solidified to a very pale yellow crystalline solid (61.2 mg; yield 92%). LC-MS analysis of the solid showed the desired product with a purity >98% and the desired product's mass: m/z 250 (M+H); Calcd for C₁₃H₁₉N₃S=249.38. ¹H NMR (400 MHz, CDCl₃): δ 1.00 (t, J=7.34 Hz, 3H, CH₃—CH₂—CH₂—), 1.36 (t, J=7.58 Hz, 3H, CH₃—CH₂—), 1.46 (dq, J=15.04 Hz and 7.38 Hz, 2H, (—CH₂—CH₃), 1.62-1.72 (m/quint, 2H, —CH₂—CH₂—), 2.57 (d, J=1.22 Hz, 3H, 5-CH₃—), 2.83 (q, J=7.66 Hz, 2H, CH₃—CH₂—), 3.64 (td, J=7.03 and 5.50 Hz, 2H, —CH₂—NH—), 5.35 (brs, 1H, —CH₂—NH), 6.72 (d, J=1.22 Hz, 1H, H-5).

### 2-Ethyl-5-methyl-N-(4,4,4-trifluorobutyl)thieno[2,3-d]pyrimidin-4-amine (1700)

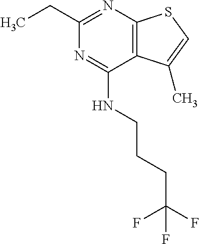 [SMILES: CCc1nc(NCCCC(F)(F)F)c2c(C)csc2n1]

A solution of 4-chloro-2-ethyl-5-methylthieno[2,3-d]pyrimidine (51.5 mg, 0.24 mmol), DIEA (75 μL, 0.44 mmol) and 4,4,4-trifluorobutan-1-amine (60 μL, 0.52 mmol) in 1,4-dioxane was heated at 140° C. in a microwave reactor for 3 h and the solvent was evaporated in vacuo to afford a pale yellow crystalline solid. The above solid was partitioned between water (25 mL) and ethyl acetate (25 mL). The organic layer was removed, washed with brine (1×10 mL) and evaporated in vacuo to afford a very pale yellow crystalline solid. The crude residue was purified by silica-gel flash chromatography to afford a very pale yellow viscous liquid which solidified to a cream crystalline solid (68.6 mg; yield 94%). LC-MS analysis of the solid showed the desired product with a purity >98% and the desired product's mass: m/z 304 (M+H); Calcd for C₁₃H₁₆F₃N₃S=303.35 ¹H NMR (400 MHz, CDCl₃): δ 1.35 (t, J=7.58 Hz, 3H), 1.94-2.05 (m, 2H), 2.16-2.32 (m, 2H), 2.58 (d, J=0.98 Hz, 3H), 2.84 (q, J=7.58 Hz, 2H), 3.73 (q, J=6.77 Hz, 2H), 5.43 (appt/brs, 1H), 6.76 (d, J=1.22 Hz, 1H). ¹⁹F NMR (376 MHz, CDCl₃): δ −66.05 (t, J=10.90 Hz, 3F, CF₃—).

### N-Cyclopropyl-2-ethyl-5-methylthieno[2,3-d]pyrimidin-4-amine (1701)

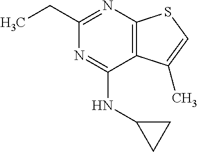 [SMILES: CCc1nc(NC2CC2)c2c(C)csc2n1]

A solution of 4-chloro-2-ethyl-5-methylthieno[2,3-d]pyrimidine (60.4 mg, 0.284 mmol), DIEA (70 μL, 0.41 mmol) and cyclopropylamine (60 μL, 0.86 mmol) in 1,4-dioxane was heated at 140° C. in a microwave reactor for 3.5 h and the solvent was evaporated in vacuo to afford an orange viscous liquid. The liquid was partitioned between water (25 mL) and ethyl acetate (25 mL). The organic layer was removed, washed with brine (1×10 mL) and evaporated in vacuo to afford an orange viscous liquid. The crude residue was purified by silica-gel flash chromatography to afford a yellow-orange viscous liquid (55.6 mg, yield 84%). LC-MS analysis of the liquid showed the desired product with a purity >98% and the desired product's mass: m/z 234 (M+H); Calcd for C₁₂H₁₅N₃S=233.33. ¹H NMR (400 MHz, DMSO-d₆): δ 0.55-0.63 (m, 2H, —CH₂—), 0.86-0.95 (m, 2H, —CH₂—), 1.38 (t, J=7.58 Hz, 3H, CH₃CH₂—), 2.52 (d, J=1.22 Hz, 3H, 5-CH₃), 2.88 (q, J=7.58 Hz, 2H, CH₃CH₂—), 2.97-3.06 (m, 1H, —CH— (cypyl)), 5.53 (brs, 1H, —NH—), 6.73 (d, J=1.22 Hz, 1H, H-6).

### 5-methyl-2-(6-methylpyridin-2-yl)-N-(3-phenylpropyl)thieno[2,3-d]pyrimidin-4-amine (1709)

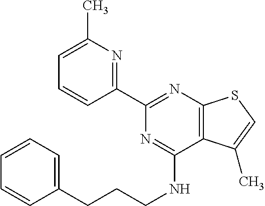 [SMILES: Cc1cccc(-c2nc(NCCCc3ccccc3)c3c(C)csc3n2)n1]

A solution of 4-chloro-5-methyl-2-(6-methylpyridin-2-yl)thieno[2,3-d]pyrimidine (48.3 mg, 0.175 mmol), DIEA (70 μL, 0.409 mmol) and 3-phenylpropan-1-amine (57.1 mg, 0.422 mmol) in 1,4-dioxane (2.00 mL) was heated at 140° C. in a microwave reactor for 3 h to give a yellow solution. LC-MS analysis of the reaction mixture showed the desired product: 5-methyl-2-(6-methylpyridin-2-yl)-N-(3-phenylpropyl)thieno[2,3-d]pyrimidin-4-amine. The solvent was evaporated in vacuo to afford a viscous yellow liquid. The material was taken up in ethyl acetate and washed with water and then brine. The layers were separated and the organic solvent was concentrated in vacuo to give a yellow solid. The crude product was purified on silica gel using ethyl acetate and hexanes as eluent to afford the desired product as a crystalline solid (58.1 mg, yield 88%). LC-MS analysis of the solid showed the desired product with a purity >98% and the desired product's mass: m/z 375 (M+H); Calcd for C₂₂H₂₂N₄S=374.34. ¹H NMR (400 MHz, CDCl₃): δ 2.12 (quin, J=7.27 Hz, 2H, —CH₂—CH₂—CH₂—), 2.56 (d, J=1.22 Hz, 3H, 5-CH₃—), 2.74 (s, 3H, 6-CH₃—Py), 2.81 (t, J=7.46 Hz, 2H, —CH₂—CH₂—CH₂-Ph), 3.76-3.85 (m/td, 2H, —NH—CH₂—CH₂—CH₂-Ph), 5.47 (t, J=5.14 Hz, 1H, —NH—), 6.87 (d, J=1.22 Hz, 1H, H-6), 7.19-7.26 (m, 4H, Ph-H and Py-H), 7.28-7.34 (m, 2H, Ph-H), 7.70 (t, J=7.70 Hz, 1H, Py-H), 8.25 (d, J=7.82 Hz, 1H).

### 5-methyl-2-(6-methylpyridin-2-yl)-N-(4,4,4-trifluorobutyl)thieno[2,3-d]pyrimidin-4-amine (1711)

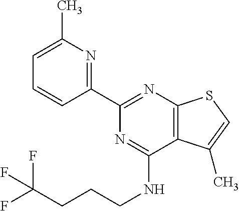 [SMILES: Cc1cccc(-c2nc(NCCCC(F)(F)F)c3c(C)csc3n2)n1]

A solution of 4-chloro-5-methyl-2-(6-methylpyridin-2-yl)thieno[2,3-d]pyrimidine (48.2 mg, 0.175 mmol), DIEA (70 μL, 0.41 mmol) and 4,4,4-trifluorobutan-1-amine (50 μL, 0.436 mmol) in 1,4-dioxane (2.00 mL) was heated at 140° C. in a microwave reactor for 3 h to give a yellow solution. LC-MS analysis of the reaction mixture showed the desired product: 5-methyl-2-(6-methylpyridin-2-yl)-N-(3-phenylpropyl)thieno[2,3-d]pyrimidin-4-amine. The solvent was evaporated in vacuo to afford a yellow residue. The material was taken up in ethyl acetate and washed with water and then brine. The layers were separated and the organic solvent was concentrated in vacuo to give a yellow solid. The crude product was purified on silica gel using ethyl acetate and hexanes as eluent to afford the desired product as a crystalline solid (59.5 mg, yield 93%). LC-MS analysis of the solid showed the desired product with a purity >98% and the desired product's mass: m/z 367 (M+H); Calcd for C₁₇H₁₇F₃N₄S=366.41. ¹H NMR (400 MHz, CDCl₃): δ 2.03-2.12 (quin, m, 2H, —CH₂—CH₂—CH₂—), 2.22-2.36 (m, 2H, —CH₂—CH₂—CH₂—), 2.64 (d, 3H, J=1.22H, 5-CH₃—), 2.74 (s, 3H, 6-CH₃—Py), 3.85 (q, J=6.77 Hz, 2H, —CH₂—CF₃), 5.57 (t, J=5.38 Hz, 1H, —NH—), 6.92 (d, J=1.22 Hz, 1H, H-6), 7.24 (d, J=7.34 Hz, Py-H-3), 7.72 (t, J=7.70 Hz, 1H, Py-H-4), 8.28 (d, J=7.58 Hz, Py-H-5).

### 2,3-dimethyl-thieno[2,3-d]pyrimidin-4-one

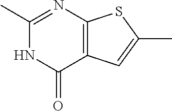

Ethyl 2-amino-5-methyl thiophene-3-carboxylate (621.4 mg, 3.355 mmol) and acetonitrile (0.352 mL, 6.7 mmol) was added to a 20 mL microwave vial. 4.0 M HCl in 1,4-dioxane (3 mL) was added to the vial to afford a yellow paste. The vial was capped and heated to 85° C. overnight while stirring. A light brown mixture formed. LC-MS analysis showed a 60% ratio of uncyclized intermediate with mass: m/z 227 (M+H), with 40% product with mass: m/z 181 (M+H). An additional 2 mL of 4.0M HCl in 1,4-dioxane was added, and the vial was heated to 110° C. overnight. LC-MS of the crude product revealed that all intermediate had converted to product. The solvent was evaporated in vacuo to afford a light-brown solid, which was suspended in acetonitrile (20 mL), heated, then filtered under vacuum to afford a light grey solid (812 mg, quantitative). LC-MS of the solid showed a purity >98% with the desired mass: m/z 181 (M+H), and m/z 203 (M+Na). Calcd for C₈H₇N₂OS=180.23. NMR (400 MHz, DMSO-d₆) δ=12.62-12.16 (m, 1H), 7.48 (s, 1H), 7.36 (s, 1H), 7.23 (s, 1H), 7.01 (d, J=1.2 Hz, 3H), 2.47 (d, J=1.0 Hz, 3H), 2.34 (s, 3H). ¹³C NMR (101 MHz, DMSO-d₆) δ=163.1, 157.6, 154.6, 135.8, 122.5, 118.9, 20.8, 15.4.

### 4-chloro-2,6-dimethylthieno[2,3-d]pyrimidine

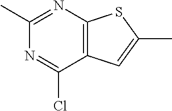

2,3-Dimethyl-thieno[2,3-d]pyrimidin-4-one (778.3 mg, 4.3 mmol) was suspended in phosphorus oxychloride (4 mL) and heated at refluxing conditions for 2 h. TLC and LC-MS showed a significant amount of starting material was still present, so the reaction mixture was heated overnight at 140° C. A dark brown mixture formed, which afforded a brown solid after drying in vacuo. POCl₃ was quenched with ice-water, then extracted with ethyl acetate. The organic layer was neutralized with a saturated NaHCO₃ solution (20 mL) and separated, then dried over anhydrous Na₂SO₄, filtered and evaporated in vacuo to afford a light brown solid. The product was dissolved in DCM and purified by silica gel chromatography (0-100% EtOAc/hexanes) to afford a white crystalline solid (471 mg, 55% yield). LC-MS analysis of the solid showed the desired product with >98% purity and mass: m/z 199 (^{35Cl}M+H) and m/z 201 (^{37Cl}M+H); Calcd for C₈H₉ClN₂S=198.67. ¹H NMR (400 MHz, DMSO-d₆) δ 7.22-7.20 (m, 1H), 2.67 (s, 3H), 2.62 (d, J=1.2 Hz, 3H)

### 2,6-dimethyl-N-(3-phenylpropyl)thieno[2,3-d]pyrimidin-4-amine (2313)

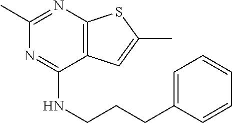 [SMILES: Cc1nc(NCCCc2ccccc2)c2cc(C)sc2n1]

A solution of 4-chloro-2,6-dimethylthieno[2,3-d]pyrimidine (10.0 mg, 0.503 mmol), 3-phenyl-n-propylamine (205 mg, 1.51 mmol), DIEA (110 μL, 0.584 mmol) in 1,4-dioxane (0.5 mL) was heated at 140° C. for 1 h in a microwave reactor. A tan solution formed, which solidified to a gel as it cooled to room temperature. The reaction mixture was partitioned between DCM and water, then the DCM layer was extracted, dried with NaSO₄, and concentrated in vacuo to afford a viscous yellow liquid. The crude product was dissolved in acetonitrile and purified by reverse phase HPLC. Again a viscous yellow liquid formed upon drying (73 mg, 48% yield). LC-MS analysis showed the desired product with >97% purity and mass: m/z 298 (M+H). Calcd for C₁₇H₁₉N₃S=297.42. ¹H NMR (400 MHz, DMSO-d₆) δ 7.33-7.15 (m, 6H), 5.75 (s, 1H), 3.51-3.43 (m, 2H), 3.33 (br. s., 6H), 2.66 (t, J=7.6 Hz, 2H), 2.40 (s, 3H), 1.95-1.85 (m, 2H) 2,6-dimethyl-N-(4,4,4-trifluorobutyl)thieno[2.3-d]pyrimidin-4-amine (2314)

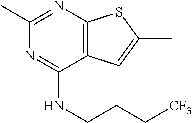 [SMILES: Cc1nc(NCCCC(F)(F)F)c2cc(C)sc2n1]

A solution of 4-chloro-2,6-dimethylthieno[2,3-d]pyrimidine (100 mg, 0.503 mmol), 4,4,4-trifluorobutylamine (175 μL, 1.53 mmol), DIEA (100 μL, 0.575 mmol) and 1,4-dioxane (0.5 mL) was heated at 140° C. for 90 min in a microwave reactor. The yellow reaction mixture was partitioned between DCM and water. The DCM layer was separated, and the aqueous layer was washed with ethyl acetate. The DCM and ethyl acetate layers were combined, dried over anhydrous NaSO₄, and concentrated to afford an off-white crystal. The crude product was purified by silica gel chromatography (0-100% EtOAc/hexanes). Upon drying, white crystals formed (141 mg, 97% yield). LC-MS analysis showed the desired product with >99% purity and mass: m/z 290 (M+H). Calcd for C₁₂H₁₄F₃N₃S=289.32. ¹H NMR (400 MHz, Chloroform-d) δ 6.72 (s, 6H), 5.06 (br. s., 6H), 3.71 (q, J=6.6 Hz, 12H), 2.59 (s, 18H), 2.54 (s, 18H), 2.29-2.14 (m, 12H), 1.95 (quin, J=7.5 Hz, 12H) 2,6-dimethyl-N-butylthieno[2,3-d]pyrimidin-4-amine (2315)

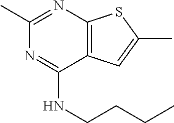 [SMILES: CCCCNc1nc(C)nc2sc(C)cc12]

A solution of 4-chloro-2,6-dimethylthieno[2,3-d]pyrimidine (100 mg, 0.503 mmol), N-butylamine (150 μL, 1.51 mmol), DIEA (100 μL, 0.575 mmol) and 1,4-dioxane (0.5 mL) was heated at 140° C. for 1 hr in a microwave reactor. Off-white crystals formed on top of a thick yellow solution. The reaction mixture was partitioned between DCM and water, then the DCM layer was separated and dried over anhydrous NaSO₄. Light orange-brown crystals formed after evaporating the solvent (98 mg, 83% yield). LC-MS analysis showed the desired product with >98% purity and mass: m/z 236.1 (M+H). Calcd for C₁₂H₁₇N₃S=235.35. ¹H NMR (400 MHz, DMSO-d₆) δ 7.54 (s, 1H), 7.20 (d, J=1.5 Hz, 1H), 3.48-3.41 (m, 2H), 2.48 (d, J=1.0 Hz, 3H), 2.39 (s, 3H), 1.61-1.52 (m, 2H), 1.40-1.30 (m, 2H), 0.91 (t, J=7.3 Hz, 3H)

### 2-methylthieno[2,3-d]pyrimidin-4-one

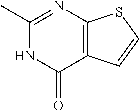

Methyl-2-aminothiophene-3-carboxylate (550 mg, 3.5 mmol) and acetonitrile (386 μL, 7.35 mmol) were combined in a large microwave vial. The vial was capped and flushed with nitrogen before adding dry 4.0M HCl in dioxane (3 mL). The solution appeared dark red. The vial was heated on a heating block at 85° C. while stirring overnight. A sticky black goo formed, which showed many impurities from polymerization by LC-MS analysis. Methanol and DCM were used to dissolve the reaction mixture, which was concentrated under vacuum to afford a black crystal residue. The compound was dissolved in methanol and purified by reverse phase HPLC (10 to 90% acetonitrile/water/0.05% TFA). The resulting solution was dried under vacuum to afford a grey solid (268 mg, 46% yield). LC-MS analysis showed the desired product with >95% purity and mass: m/z 167 (M+H). Calcd for C₇H₆N₂OS=166.20. ¹H NMR (400 MHz, Chloroform-d) δ 12.42-12.00 (m, 1H), 7.48 (d, J=5.9 Hz, 1H), 7.24 (d, J=5.9 Hz, 1H), 2.61 (s, 3H)

### 4-chloro-2-methylthieno[2,3-d]pyrimidine

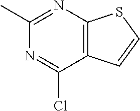

2-methylthieno[2,3-d]pyrimidin-4-one (241 mg, 1.45 mmol) and phosphorus oxychloride (3 mL, 32 mmol) were combined in a round bottom flask and heated at refluxing conditions (115° C.) overnight. An amber solution formed. The POCl₃ was evaporated in vacuo, then quenched over ice-water. The product crashed out into solution and was filtered by vacuum, then purified by silica gel chromatography (0-100% EtOAc/hexanes). After drying under vacuum a white crystal formed (118 mg, 44% yield). LC-MS analysis showed the desired product with >98% purity and mass: m/z 185 (^{35Cl}M+H) and m/z 187 (^{37Cl}M+H). Calcd for C₇H₅ClN₂S=184.64. ¹H NMR (400 MHz, DMSO-d₆) δ 7.46 (d, J=5.9 Hz, 1H), 7.32 (d, J=5.4 Hz, 1H), 2.36 (s, 3H)

### 2-methyl-N-(3-phenylpropyl)thieno[2,3-d]pyrimidin-4-amine (2316)

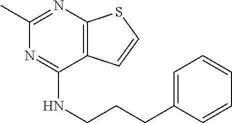 [SMILES: Cc1nc(NCCCc2ccccc2)c2ccsc2n1]

A clear solution of 4-chloro-2-methylthieno[2,3-cl]pyrimidine (30 mg, 0.162 mmol), 3-phenylpropylamine (67 mg, 0.50 mmol), DIEA (35 μL, 0.20 mmol), and dry p-dioxane (0.5 mL) was heated at 140° C. for 90 minutes in a microwave reactor. A tan liquid formed, which was partitioned between DCM and water. The DCM layer was separated and dried over anhydrous NaSO₄ before concentrating under vacuum. The product was purified by silica gel chromatography (0-100% EtOAc/hexanes) to yield the title compound as a viscous yellow substance (35 mg, 76% yield). LC-MS showed the desired product with >95% purity and mass: m/z 284.1 (M+H). Calcd for C₁₆H₁₇N₃S=283.39. ¹H NMR (400 MHz, CHLOROFORM-d) δ 7.36-7.22 (m, 19H), 7.17 (d, J=5.9 Hz, 4H), 6.96 (d, J=5.9 Hz, 4H), 5.11 (br. s., 1H), 3.75-3.68 (m, 2H), 2.80 (t, J=7.5 Hz, 8H), 2.64 (s, 3H), 2.08 (quin, J=7.2 Hz, 2H)

### 2-methyl-N-(4,4,4-trifluorobutyl)thieno[2.3-d]pyrimidin-4-amine (2317)

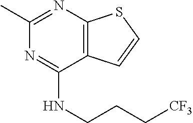 [SMILES: Cc1nc(NCCCC(F)(F)F)c2ccsc2n1]

A clear solution of 4-chloro-2-methylthieno[2,3-d]pyrimidine (30 mg, 0.162 mmol), 4,4,4-trifluorobutylamine (55 μL, 0.486 mmol), DIEA (35 μL, 0.20 mmol), and dry p-dioxane (0.5 mL) was heated at 140° C. for 90 minutes in a microwave reactor. A yellow solution formed, which was partitioned between DCM and water. The DCM layer was separated and set aside, and the aqueous layer was washed with ethyl acetate. Both organic layers were combined and concentrated under vacuum to afford a viscous yellow substance, which was lyophilized for 2 hours to give the final product (37 mg, 83% yield). LC-MS analysis indicated the desired product with >97% purity and mass: m/z 276 (M+H). Calcd for C₁₁H₁₂F₃N₃S=275.29. ¹H NMR (400 MHz, DMSO-d₆) δ 7.87 (t, J=5.4 Hz, 4H), 7.53 (d, J=5.9 Hz, 5H), 7.43 (d, J=5.9 Hz, 1H), 3.60-3.51 (m, 10H), 2.43 (s, 3H), 2.40-2.30 (m, 9H), 1.93-1.74 (m, 10H).

### D. Biological Activity

The present compounds have an activity as shown in Table 1 below.

TABLE 2
Activity of Compounds Against Mtb

| Example | MABA MIC90 | MABA MIC50 | Disk Zone of |
|---|---|---|---|
| No. | (μM) | (μM) | Inhibition (mm) |
| 0728 | 0.524 | 0.182 | 31, 11 |
| 0795 | 28.4 | 25.4 | 0 |
| 0796 | >50.0 | >50.0 | 6 |
| 0797 | >50.0 | >50.0 | 0 |
| 0798 | 32.9 | 27.4 | 1.5 |
| 0851 | >50.0 | >50.0 | 0 |
| 0881 | 13.4 | 6.45 | 7 |
| 0927 | 4.02 | 1.77 | 10 |
| 0935 | >50.0 | >50.0 | |
| 0936 | 48.2 | 26 | 0.75 |
| 0941 | 16.3 | 7.79 | 4 |
| 0942 | >50.0 | 47.5 | 5 |
| 0943 | >50.0 | >50.0 | 4 |
| 0946 | 2.04 | 0.633 | 27, 20 |
| 0950 | 28.4 | 15.9 | 0 |
| 0951 | >50.0 | >50.0 | 1 |
| 1019 | 26.4 | 11.1 | 10 |
| 1020 | 0.163 | 0.116 | 25 |
| 1021 | 9.81 | 3.93 | 10 |
| 1022 | 0.96 | 0.329 | 14 |
| 1023 | 0.108 | 0.0266 | 13 |
| 1060 | 17.2 | 10 | 0 |
| 1061 | 1.28 | 0.615 | 19 |
| 1062 | 6.01 | 2.88 | 0 |
| 1063 | 41.7 | 21 | 4 |
| 1069 | >50.0 | 30.9 | 5 |
| 1072 | 19.2 | 6.32 | 9 |
| 1074 | 22.7 | 6.1 | 21 |
| 1142 | 24.8 | 8.37 | |
| 1146 | 13.8 | 5.98 | |
| 1150 | 23.1 | 21.1 | |
| 1151 | 33.9 | 11.1 | |
| 1154 | >50.0 | 45.9 | |
| 1161 | 36.3 | 31.3 | |
| 1176 | 14.4 | 3.8 | |
| 1177 | >50.0 | 30.8 | |
| 1181 | 7.64 | 3.06 | |
| 1299 | 0.188 | 0.128 | |
| 1300 | 0.389 | 0.164 | |
| 1301 | 8.97 | 3.8 | |
| 1302 | 5.54 | 2.63 | |
| 1303 | 20.8 | 10.6 | |
| 1304 | 41.6 | 25.2 | |
| 1305 | 0.145 | 0.113 | |
| 1306 | 0.183 | 0.125 | |
| 1307 | 1.18 | 0.526 | |
| 1308 | 31.6 | 21.6 | |
| 1366 | 0.00369 | 0.00133 | 14 |
| 1536 | 0.479 | 0.386 | 2.7 |
| 1537 | 1.14 | 0.439 | 1.5 |
| 1538 | 1.1 | 0.597 | 1.2 |
| 1539 | 7.36 | 4.9 | <1.0 |
| 1540 | 4.79 | 1.81 | 2.7 |
| 1541 | >50.0 | 36.8 | 1.8 |
| 1542 | 10.1 | 5.28 | 6.2 |
| 1543 | 29.6 | 26.9 | 9.3 |
| 1544 | 3.69 | 1.14 | 5 |
| 1545 | 30.5 | 26.9 | 1.8 |
| 1546 | 12.4 | 11.2 | 3.3 |
| 1547 | 23.8 | 21.9 | 9.7 |
| 1548 | 13.7 | 12.3 | 9 |
| 1549 | 24.2 | 22.3 | 6.7 |
| 1551 | 3.88 | 0.846 | 6.2 |
| 1698 | 3.67 | 0.515 | 6.8 |
| 1699 | 45.5 | 35.5 | 6 |
| 1700 | 30.7 | 26.8 | 6 |
| 1701 | 16.1 | 9.77 | 1.7 |
| 1709 | 19.7 | 2.56 | |
| 1711 | 19.4 | 6.48 | |

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### V. REFERENCES

The following references, to the extent that they provide exemplary procedural or other details supplementary to those set forth herein, are specifically incorporated herein by reference:
WHO. 2015. Global Tuberculosis Report. www.who.int/tb/publications/global_report/en/1-97.
Medlar, “The behavior of pulmonary tuberculous lesions; a pathological study,” Am. Rev. Tuberc., 71:1-244, 1955.
Golub et al., “Synthesis and biological evaluation of substituted (thieno[2,3-d]pyrimidin-4-ylthio)carboxylic acids as inhibitors of human protein kinase CK2,” Eur. J. Med. Chem., 46:870-876, 2011.
Raghu et al., “Microwave-Assisted Synthesis of Novel-5-Substituted-2,3-dihydroimidazo[1,2-c]thieno[3,2-e]pyrimidine s,” Synthesis (Stuttg), 2119-2123, 2001.
Adib et al., “One-Pot Four-Component Synthesis of Thieno[2,3-d]pyrimidin-4-amines via Sequential Gewald/Cyclocondensation Reactions,” Helv. Chim. Acta., 98:1079-1086, 2015.
Wang et al., “Synthesis and Evaluation of Biological and Antitumor Activities of Tetrahydrobenzothieno[2,3-d]Pyrimidine Derivatives as Novel Inhibitors of FGFR1,” Chem. Biol. Drug Des., 87:499-507, 2016.
Yang et al., “Synthesis and Crystal Structure of 4-[5-(2-Bromophenyl)-1,3,4-Thiadiazol-2-Ylthio]-2-(trifluoromethyl)thieno[2,3-d]pyrimidine,” Advanced Materials Research, 887-888:703-706, 2014.
Franzblau et al., “Comprehensive analysis of methods used for the evaluation of compounds against Mycobacterium tuberculosis,” Tuberculosis (Edinb), 92:453-488, 2012.

What is claimed is:

1. A compound of the formula: 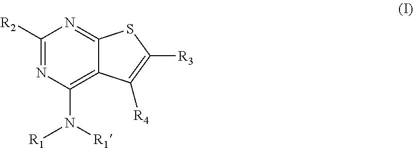
wherein:
R₁ is —(CH₂)ₓRₐ;
R₁′ is hydrogen, alkyl_{(C≤8)}, or substituted alkyl_{(C≤8)}, or —(CH₂)ₓRₐ, wherein:
x is 3, 4, or 5;
Rₐ is aryl_{(C≤12)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 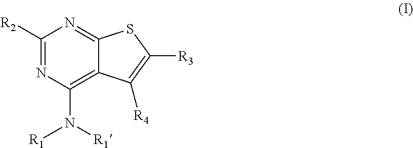
wherein:
R₁ is substituted aralkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 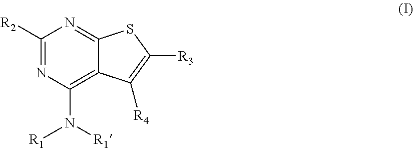
wherein:
R₁ and are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is hydrogen, alkyl_{(C≤12)}, aryl_{(C≤12)}, heteroaryl_{(C≤12)}, aralkyl_{(C≤12)}, heteroaralkyl_{(C≤12)}, or a substituted version of the last five groups;
R₃ is halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is hydrogen, alkyl_{(C≤6)}, or substituted alkyl_{(C≤6)}; or
a compound of the formula: 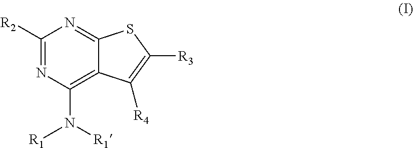
wherein:
R₁ is haloalkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 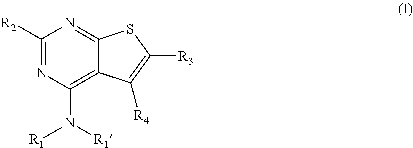
wherein:
R₁ is branched alkyl_{(C≤12)} or substituted branched alkyl_{(C≤8)};
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 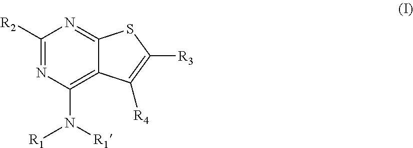
wherein:
R₁ and R₁′ is hydrogen, alkyl_{(C≤8)}, substituted alkyl_{(C≤8)}, aralkyl_{(C≤8)}, or substituted aralkyl_{(C≤8)};
R₂ is branched alkyl_{(C≤8)} or substituted branched alkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 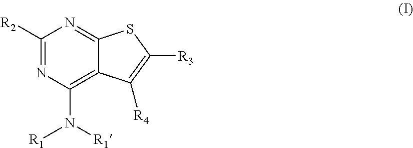
wherein:
R₁ and R₁′ is hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is haloalkyl_{(C≤8)} or substituted haloalkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)}; or
a compound of the formula: 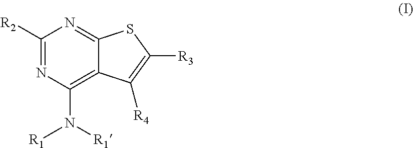
wherein:
R₁ and R₁′ are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is heteroaryl_{(C≤12)}, heteroaralkyl_{(C≤12)}, or a substituted version of either group;
R₃ is hydrogen, halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is hydrogen, alkyl_{(C≤6)}, or substituted alkyl_{(C≤6)}; or
a compound of the formula: 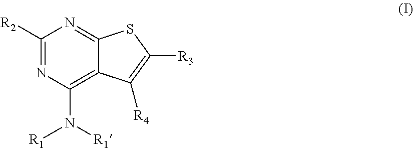
wherein:
R₁ and R₁′ are each independently hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is hydrogen, alkyl_{(C≤12)}, aryl_{(C≤12)}, heteroaryl_{(C≤12)}, aralkyl_{(C≤12)}, heteroaralkyl_{(C≤12)}, or a substituted version of the last five groups;
R₃ is hydrogen, halo, substituted alkyl_{(C≤12)}, cycloalkyl_{(C≤12)}, substituted cycloalkyl_{(C≤12)}, alkenyl_{(C≤12)}, substituted alkenyl_{(C≤12)}, aryl_{(C≤12)}, substituted aryl_{(C≤12)}, aralkyl_{(C≤12)}, or substituted aralkyl_{(C≤12)}; and
R₄ is alkyl_{(C≤6)} or substituted alkyl_{(C≤6)}; or
a compound of the formula: 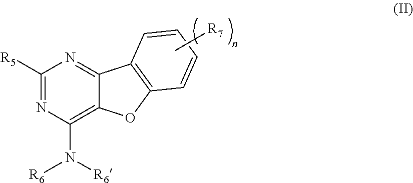
wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c}, wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
provided that when R₅ is methyl and n is 0, then R₆ is not butyl when R₆′ is hydrogen;
or a pharmaceutically acceptable salt thereof.

2. The compound of claim 1 further defined as: 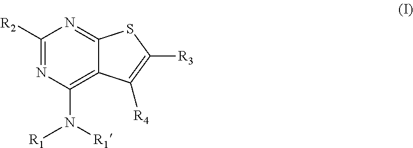
wherein:
R₁ is —(CH₂)ₓRₐ;
R₁′ is hydrogen, alkyl_{(C≤8)}, or substituted alkyl_{(C≤8)}, or —(CH₂)ₓRₐ, wherein:
x is 3, 4, or 5;
Rₐ is aryl_{(C≤12)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
or a pharmaceutically acceptable salt thereof.

3. The compound of claim 1 further defined as: 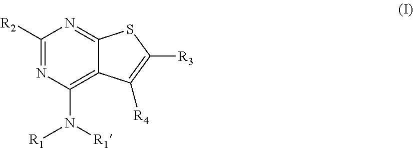
wherein:
R₁ is haloalkyl_{(C≤12)},
R₁′ is hydrogen, alkyl_{(C≤8)}, substituted alkyl_{(C≤8)}, aralkyl_{(C≤8)}, or substituted aralkyl_{(C≤8)};
R₂ is hydrogen, alkyl_{(C≤12)}, aryl_{(C≤12)}, aralkyl_{(C≤12)}, or a substituted version of the last three groups;
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
or a pharmaceutically acceptable salt thereof.

4. The compound of claim 1 further defined as: 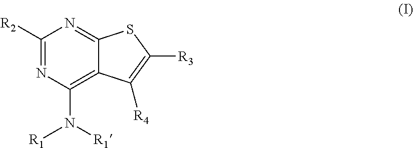
wherein:
R₁ and R₁′ is hydrogen, alkyl_{(C≤8)}, substituted alkyl_{(C≤8)}, cycloalkyl_{(C≤8)}, substituted cycloalkyl_{(C≤8)}, aralkyl_{(C≤8)}, or substituted aralkyl_{(C≤8)};
R₂ is haloalkyl_{(C≤8)};
R₃ is hydrogen, halo, alkyl_{(C≤12)}, cycloalkyl_{(C≤12)}, alkenyl_{(C≤12)}, aryl_{(C≤12)}, aralkyl_{(C≤12)}, or a substituted version of the last five groups; and
R₄ is hydrogen, alkyl_{(C≤6)}, or substituted alkyl_{(C≤6)};
or a pharmaceutically acceptable salt thereof.

5. The compound of claim 1 further defined as: 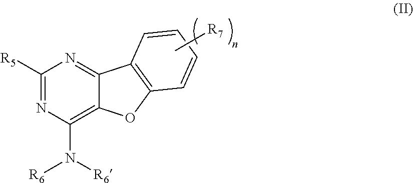
wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c}, wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
provided that when R₅ is methyl and n is 0, then R₆ is not butyl when R₆′ is hydrogen;
or a pharmaceutically acceptable salt thereof.

6. The compound of claim 5 further defined as: 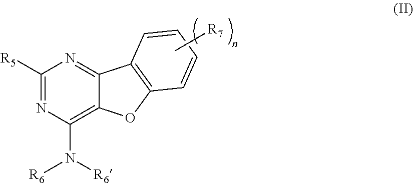
wherein:
R₅ is aryl_{(C≤12)}, aralkyl_{(C≤12)}, or a substituted version of either of these groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —R_{b}OR_{c}, wherein R_{b} is alkanediyl_{(C≤8)} or substituted alkanediyl_{(C≤8)} and R_{c} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
or a pharmaceutically acceptable salt thereof.

7. The compound according to any one of claim 1-3, wherein R₂ is alkyl_{(C1-3)}.

8. The compound of claim 7, wherein R₂ is methyl or ethyl.

9. The compound according to any one of claims 1-4, wherein R₂ is trifluoromethyl or pentafluoroethyl.

10. The compound according to any one of claims 1-3, 7, 8, and 9, wherein R₄ is hydrogen.

11. The compound according to any one of claims 1-3 and 7-10, wherein R₁ is hydrogen or methyl.

12. The compound according to any one of claims 1-3 and 7-10, wherein R₁ is halo.

13. The compound according to any one of claims 1, 3, and 7-12, wherein R₁′ is 4,4,4-trifluorobutyl.

14. The compound according to any one of claims 1, 2, and 7-12, wherein x is 3.

15. The compound according to any one of claims 1, 2, 7-12, and 14, wherein Rₐ is phenyl.

16. The compound according to any one of claims 1, 5, and 6, wherein R₅ is alkyl_{(C1-3)} or substituted alkyl_{(C1-3)}.

17. The compound of claim 16, wherein R₅ is methyl or ethyl.

18. The compound according to any one of claims 1, 5, 6, 16, and 17, wherein n is 0.

19. The compound according to any one of claims 1, 5, 6, and 16-18, wherein R₆ is aralkyl_{(C≤12)} or substituted aralkyl_{(C≤12)}.

20. The compound of claim 19, wherein R₆ is 3-phenylpropyl.

21. The compound according to any one of claims 1-6, wherein the compound is further defined as: 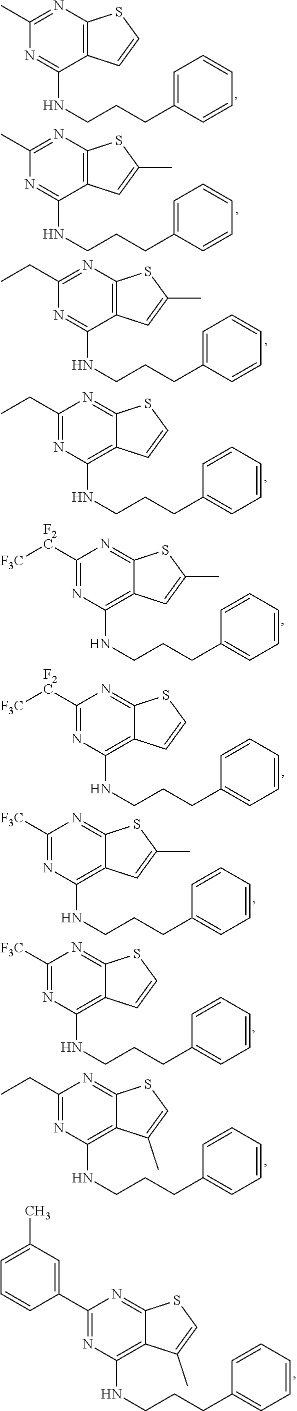 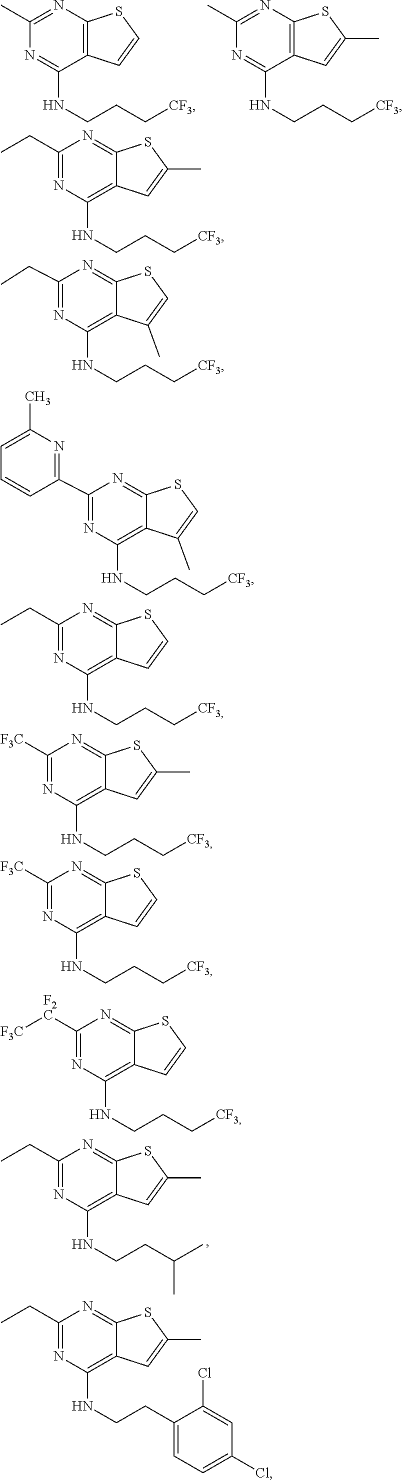 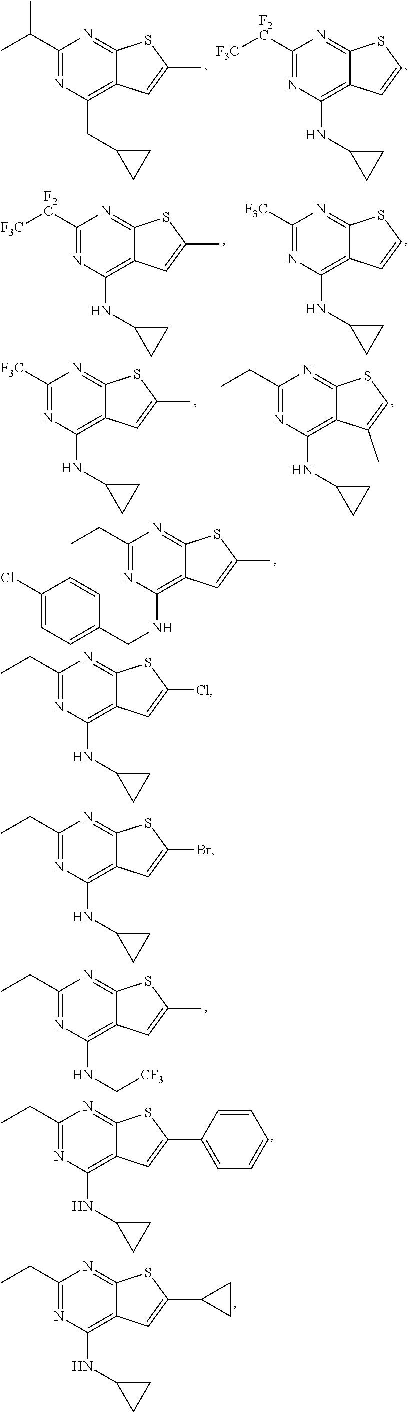 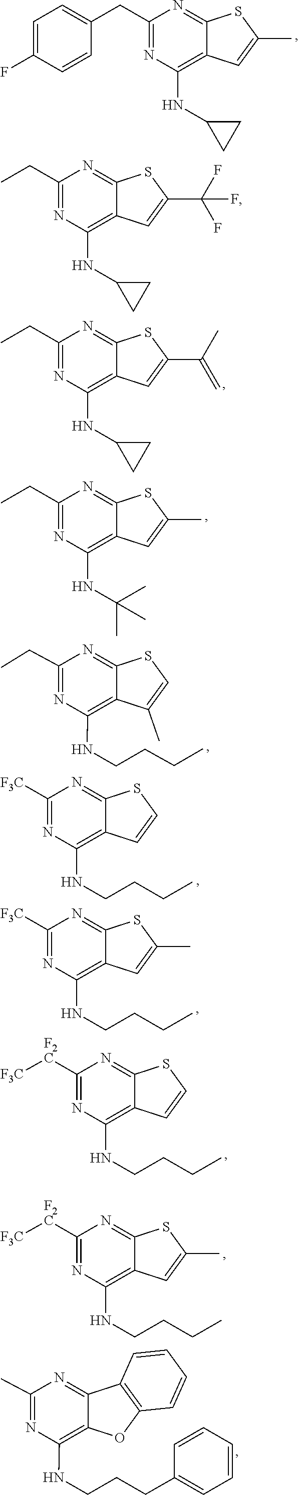 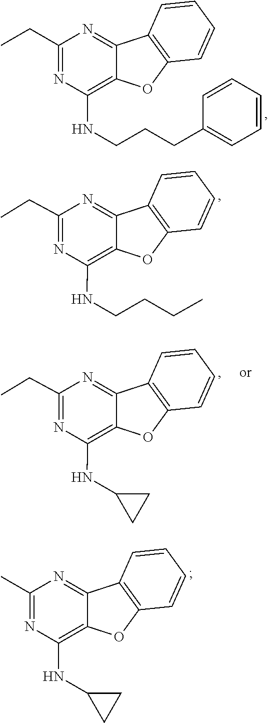
or a pharmaceutically acceptable salt thereof.

22. A compound of the formula: 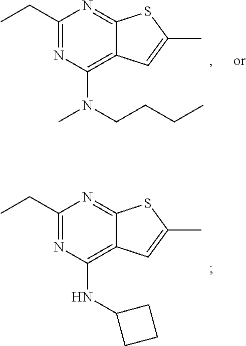
or a pharmaceutically acceptable salt thereof.

23. A pharmaceutical composition comprising:
(A) a compound according to any one of claims 1-22; and
(B) an excipient.

24. The pharmaceutical composition of claim 23, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

25. A method of treating tuberculosis in a patient comprising administering to the patient a therapeutically effective amount of a compound of the formula: 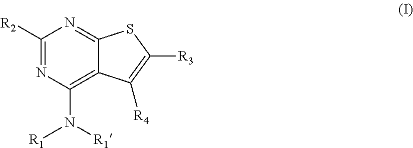
wherein:
R₁ is hydrogen, alkyl_{(C≤12)}, substituted alkyl_{(C≤12)}, cycloalkyl_{(C≤12)}, substituted cycloalkyl_{(C≤12)}, aralkyl_{(C≤12)}, or substituted aralkyl_{(C≤12)};
R₁′ is hydrogen or alkyl_{(C≤12)}, cycloalkyl_{(C≤12)}, aralkyl_{(C≤18)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these six groups;
R₃ is hydrogen, halo, or alkyl_{(C≤12)}, cycloalkyl_{(C≤12)}, alkenyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
a compound of the formula: 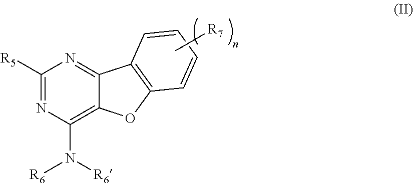
wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
or a pharmaceutically acceptable salt thereof;
provided that the compound is not:

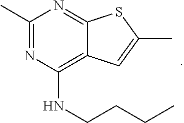 [SMILES: CCCCNc1nc(C)nc2sc(C)cc12]

26. The method of claim 25, wherein the compound is further defined as a compound of formula I.

27. The method of either claim 25 or claim 26, wherein R₁ is hydrogen.

28. The method according to any one of claims 25-27, wherein R₁′ is alkyl_{(C≤8)} or substituted alkyl_{(C≤8)}.

29. The method of claim 28, wherein R₁′ is alkyl_{(C≤8)}.

30. The method of claim 29, wherein R₁′ is n-butyl or 3-methylbutyl.

31. The method of claim 28, wherein R₁′ is substituted alkyl_{(C≤8)}.

32. The method of claim 31, wherein R₁′ is 4,4,4-trifluorobutyl.

33. The method according to any one of claims 25-27, wherein R₁′ is cycloalkyl_{(C≤8)} or substituted cycloalkyl_{(C≤8)}.

34. The method of claim 33, wherein R₁′ is cycloalkyl_{(C≤8)}.

35. The method of claim 34, wherein R₁′ is cyclopropyl.

36. The method according to any one of claims 25-27, wherein R₁′ is aralkyl_{(C≤12)} or substituted aralkyl_{(C≤12)}.

37. The method of claim 36, wherein R₁′ is aralkyl_{(C≤12)}.

38. The method of claim 37, wherein R₁′ is 3-phenylpropyl.

39. The method according to any one of claims 25-38, wherein R₂ is alkyl_{(C≤8)}.

40. The method of claim 39, wherein R₂ is methyl, ethyl, or isopropyl.

41. The method according to any one of claims 25-38, wherein R₂ is fluoroalkyl_{(C≤8)}.

42. The method of claim 40, wherein R₂ is trifluoromethyl or pentafluoroethyl.

43. The method according to any one of claims 25-38, wherein R₂ is aryl_{(C≤8)}.

44. The method of claim 43, wherein R₂ is phenyl.

45. The method according to any one of claims 25-38, wherein R₂ is aralkyl_{(C≤8)}.

46. The method of claim 45, wherein R₂ is benzyl.

47. The method according to any one of claims 25-46, wherein R₃ is hydrogen.

48. The method according to any one of claims 25-46, wherein R₃ is halo.

49. The method of claim 48, wherein R₃ is chloro.

50. The method according to any one of claims 25-46, wherein R₃ is alkyl_{(C≤8)}.

51. The method of claim 50, wherein R₃ is methyl.

52. The method according to any one of claims 25-51, wherein R₄ is hydrogen.

53. The method of claim 25, wherein the compound is further defined as a compound of formula II.

54. The method of either claim 25 or claim 53, wherein R₅ is alkyl_{(C≤8)} or substituted alkyl_{(C≤8)}.

55. The method of claim 54, wherein R₅ is alkyl_{(C≤8)}.

56. The method of claim 55, wherein R₅ is methyl or ethyl.

57. The method according to any one of claims 25 and 53-56, wherein R₆ is hydrogen.

58. The method according to any one of claims 25 and 53-57, wherein R₆′ is alkyl_{(C≤8)}.

59. The method of claim 58, wherein R₆′ is butyl.

60. The method according to any one of claims 25 and 53-57, wherein R₆′ is cycloalkyl_{(C≤8)}.

61. The method of claim 60, wherein R₆′ is cyclopropyl.

62. The method according to any one of claims 25 and 53-57, wherein R₆′ is aralkyl_{(C≤8)}.

63. The method of claim 62, wherein R₆′ is 3-phenylpropyl.

64. The method according to any one of claims 25-63, wherein the compound is further defined as: 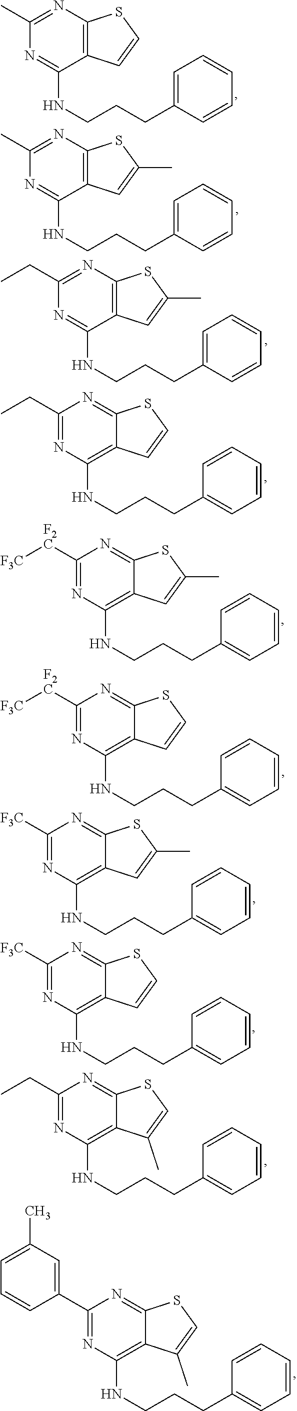 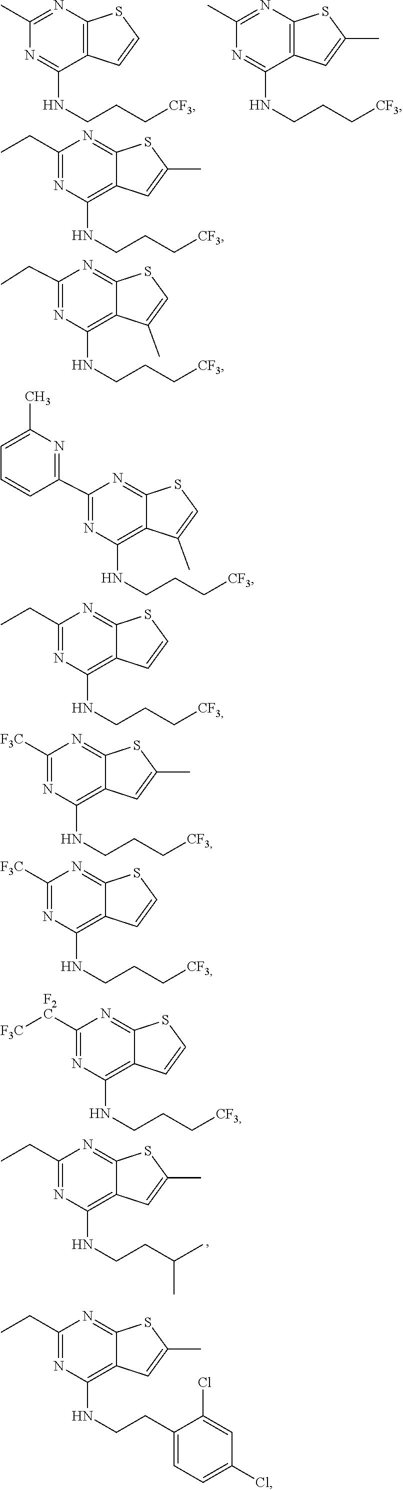 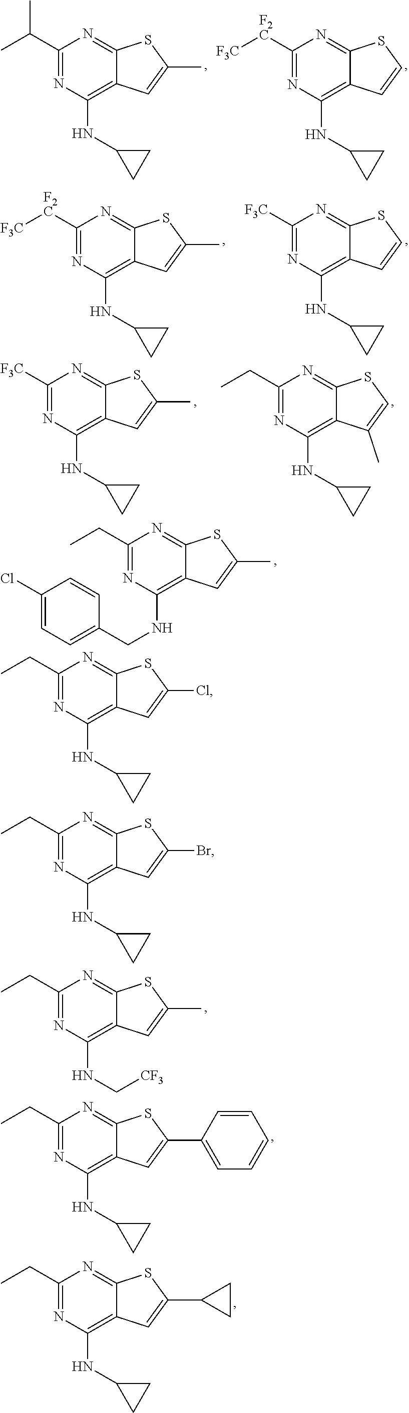 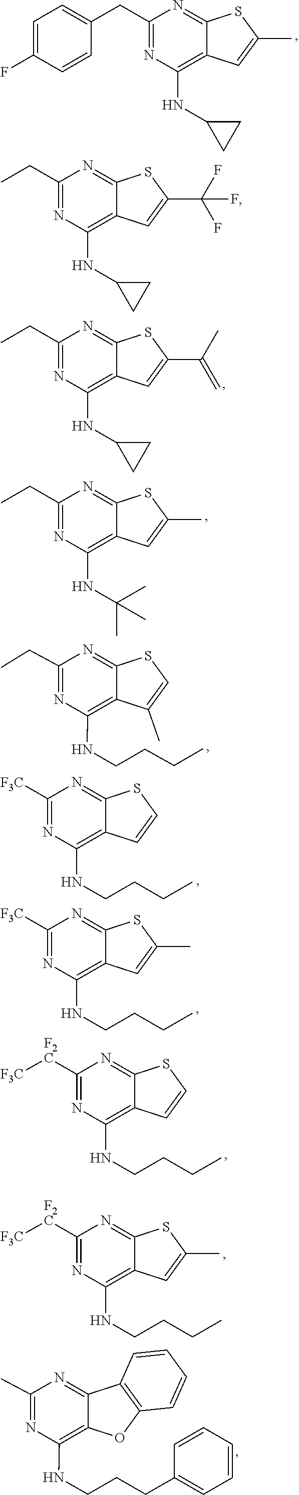 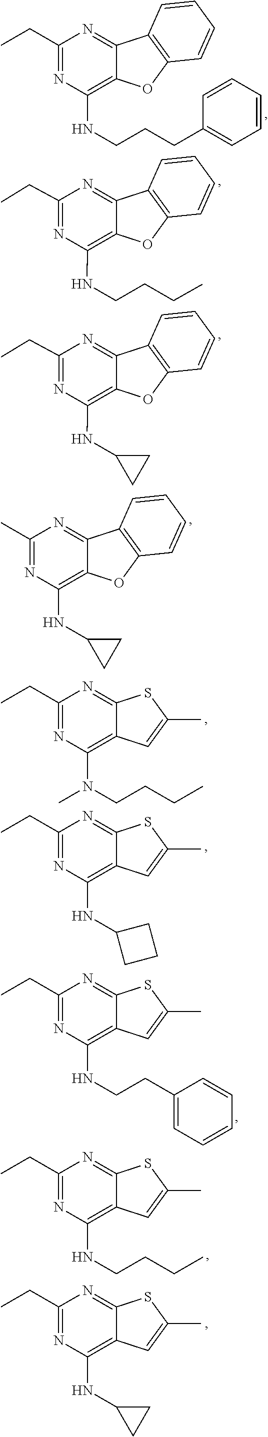 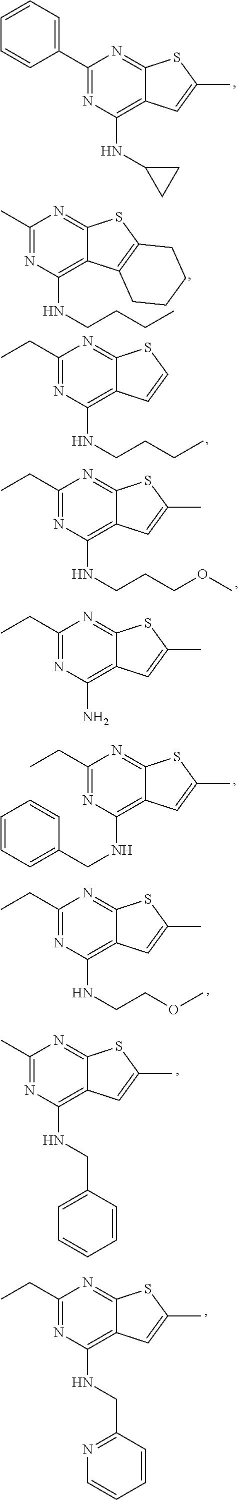 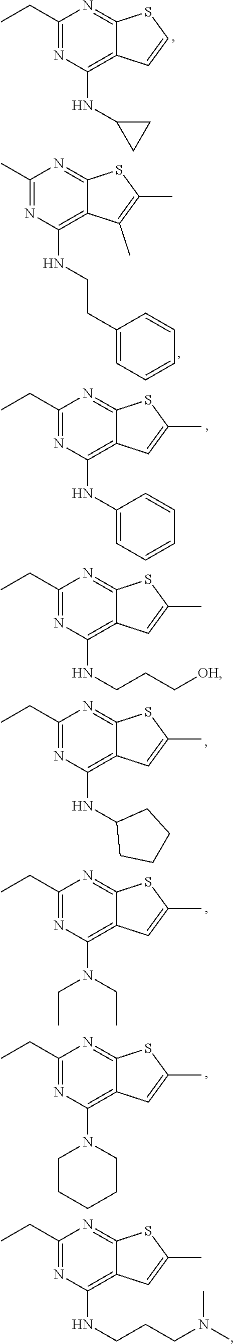 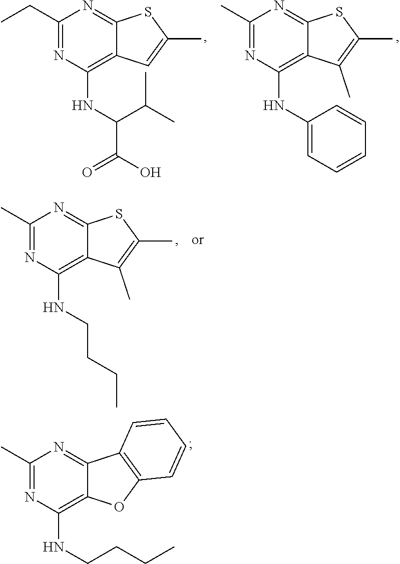
or a pharmaceutically acceptable salt thereof.

65. The method according to any one of claims 25-64, wherein the compound is formulated as a pharmaceutical composition and further comprises an excipient.

66. The method of claim 65, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularly, intravitreally, liposomally, locally, mucosally, parenterally, rectally, subconjunctivally, subcutaneously, sublingually, topically, transbuccally, transdermally, vaginally, in crèmes, in lipid compositions, via a catheter, via a lavage, via continuous infusion, via infusion, via inhalation, via injection, via local delivery, or via localized perfusion.

67. The method according to any one of claims 25-66, wherein the tuberculosis is caused by a multi-drug resistant mycobacteria.

68. The method according to any one of claims 25-66, wherein the tuberculosis is caused by a extensively drug resistant mycobacteria.

69. The method according to any one of claims 25-68, wherein the patient is a mammal.

70. The method of claim 69, wherein the patient is a human.

71. The method according to any one of claims 25-70, wherein the method further comprises a second anti-tuberculosis therapy.

72. The method of claim 71, wherein the second anti-tuberculosis therapy is a first line anti-tuberculosis therapy.

73. The method of claim 72, wherein the first line anti-tuberculosis therapy is ethambutol, isoniazid, pyrazinamide, rifampicin, or streptomycin.

74. The method of claim 71, wherein the second anti-tuberculosis therapy is a second line anti-tuberculosis therapy.

75. The method of claim 74, wherein the second line anti-tuberculosis therapy is an aminoglycoside, a polypeptide antibiotic, a fluoroquinolone, a thioamide, cycloserine, or terizidone.

76. The method of claim 75, wherein the aminoglycoside is amikacin or kanamycin.

77. The method of claim 75, wherein the polypeptide antibiotic is capreomycin, viomycin, or enviomycin.

78. The method of claim 75, wherein the fluoroquinolone is ciprofloxacin, levofloxacin, or moxifloxacin.

79. The method of claim 75, wherein the thioamide is ethionamide or prothionamide.

80. The method of claim 71, wherein the second anti-tuberculosis therapy is a third line anti-tuberculosis therapy.

81. The method of claim 80, wherein the third line anti-tuberculosis therapy is rifabutin, a macrolide, linezolid, thioacetazone, thioridazine, arginine, vitamin D, or bedaquiline.

82. The method of claim 81, wherein the macrolide is clarithromycin.

83. The method according to any one of claims 71-82, wherein the second anti-tuberculosis therapy further comprises 1, 2, 3, or 4 additional anti-tuberculosis therapies.

84. The method of claim 83, wherein method further comprises administering the compound or pharmaceutical composition in combination with ethambutol, isoniazid, rifamycin, and pyrazinamide.

85. The method according to any one of claims 25-84, wherein the compound or the pharmaceutical composition is administered once.

86. The method according to any one of claims 25-84, wherein the compound or the pharmaceutical composition is administered two or more times.

87. A method of inducing the death of a Mycobacterium tuberculosis bacterium comprising contacting the bacteria with an effective amount of a compound of the formula: 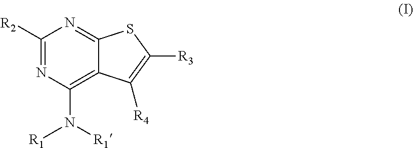
wherein:
R₁ is hydrogen, alkyl_{(C≤12)}, substituted alkyl_{(C≤12)}, cycloalkyl_{(C≤12)}, substituted cycloalkyl_{(C≤12)}, aralkyl_{(C≤12)}, or substituted aralkyl_{(C≤12)};
R₁′ is hydrogen or alkyl_{(C≤12)}, cycloalkyl_{(C≤12)}, aralkyk_{(C≤18)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, heteroaryl_{(C≤12)}, aralkyl_{(C≤12)}, heteroaralkyl_{(C≤12)}, or a substituted version of any of these six groups;
R₃ is hydrogen, halo, or alkyl_{(C≤12)}, cycloalkyl_{(C≤12)}, alkenyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
a compound of the formula: 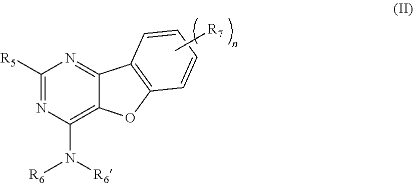
wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
or a pharmaceutically acceptable salt thereof;
provided that the compound is not:

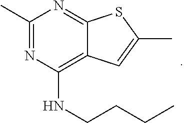 [SMILES: CCCCNc1nc(C)nc2sc(C)cc12]

88. The method of claim 87, wherein the compound is further defined as: 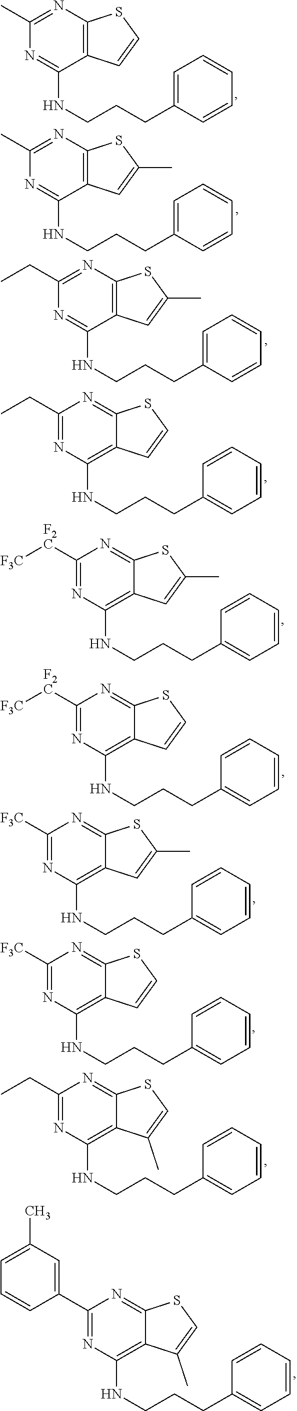 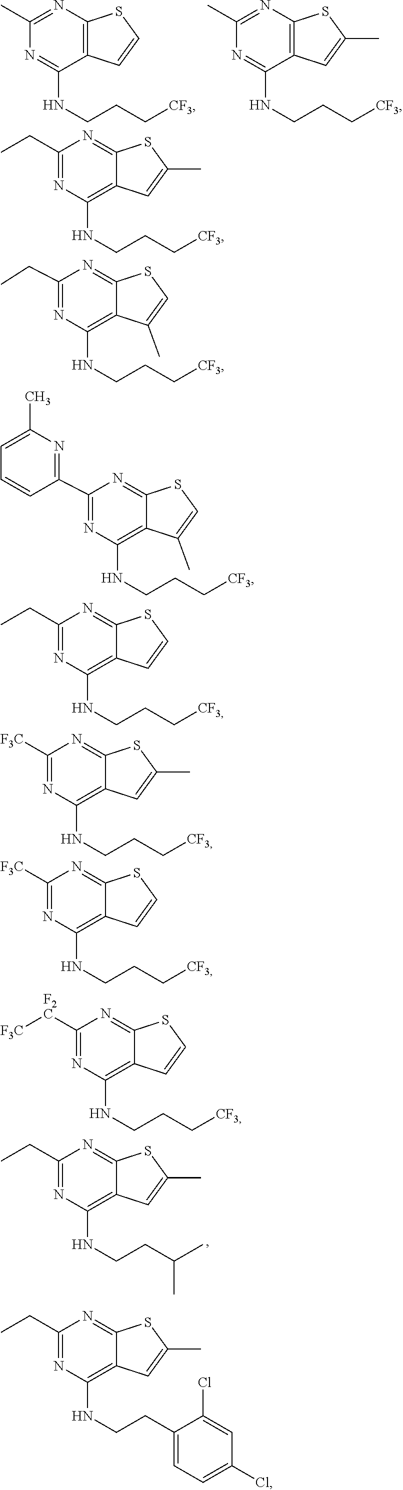 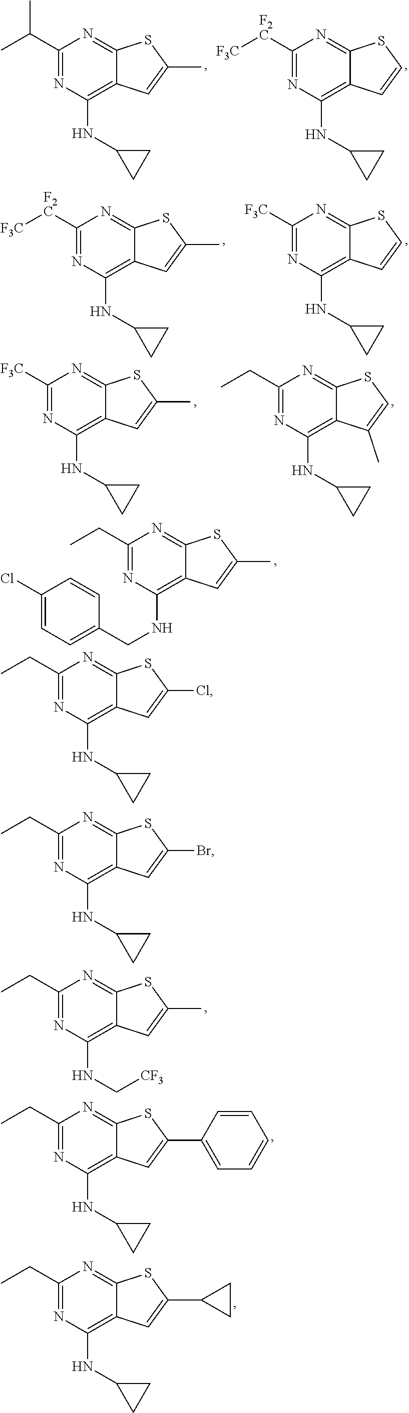 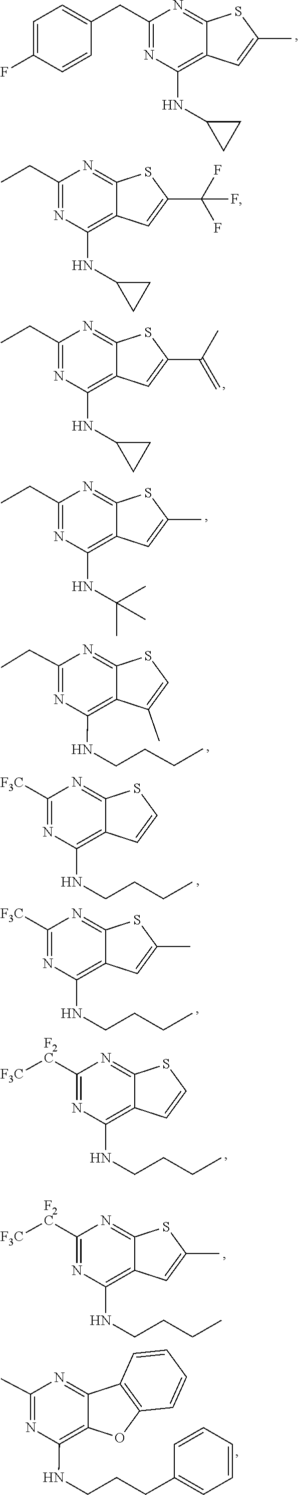 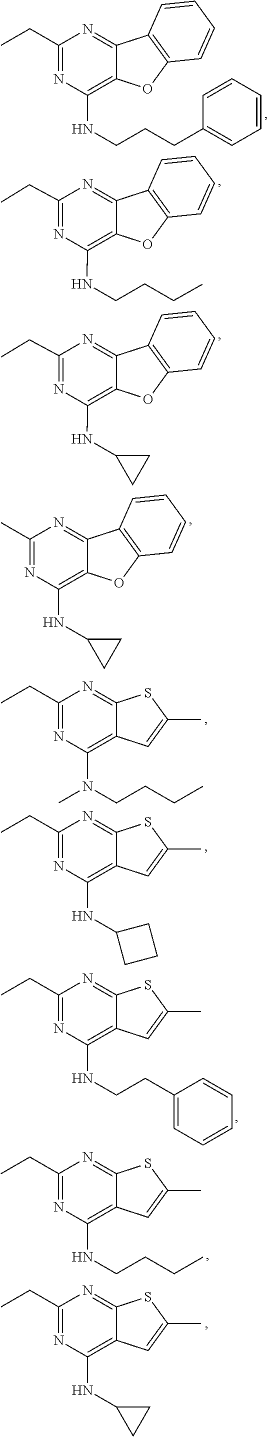 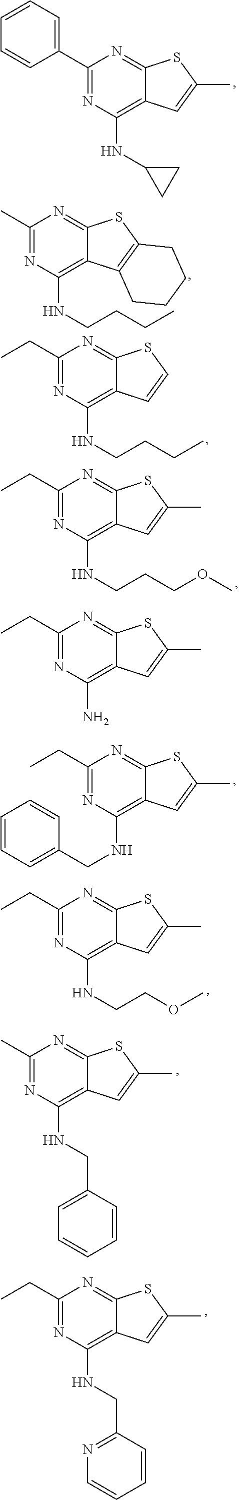 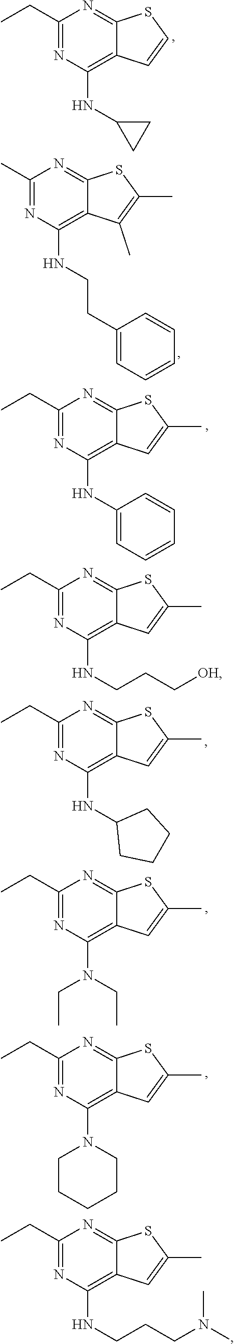 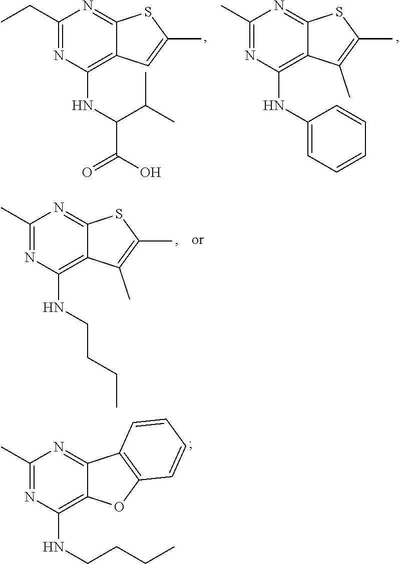
or a pharmaceutically acceptable salt thereof.

89. The method of either claim 87 or claim 88, wherein the method is sufficient to treat a Mycobacterium tuberculosis infection in a patient.

90. A method of inhibiting the replication of a Mycobacterium tuberculosis bacterium comprising contacting the bacteria with an effective amount of a compound of the formula: 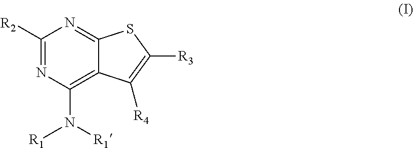
wherein:
R₁ is hydrogen, alkyl_{(C≤12)}, substituted alkyl_{(C≤12)}, cycloalkyl_{(C≤12)}, or substituted cycloalkyl_{(C≤12)};
R₁′ is hydrogen or alkyl_{(C≤12)}, cycloalkyl_{(C≤12)}, aralkyl_{(C≤12)}, or a substituted version of these three groups; or —R_{d}ORₑ, wherein R_{d} is alkanediyl_{(C≤8)} or substituted alkanediyl_{(C≤8)} and Rₑ is alkyl_{(C≤8)}, cycloalkyl_{(C≤8)}, or a substituted version of either group;
R₁ and R₁′ are taken together and are alkanediyl_{(C≤8)} or substituted alkanediyl_{(C≤8)};
R₂ is hydrogen or alkyl_{(C≤12)}, cycloalkyl_{(C≤12)}, aryl_{(C≤12)}, aralkyl_{(C≤12)}, or a substituted version of any of these four groups;
R₃ is hydrogen, halo, or alkyl_{(C≤12)}, cycloalkyl_{(C≤12)}, aryl_{(C≤12)}, aralkyl_{(C≤12)}, or a substituted version of any of these four groups; and
R₄ is hydrogen, alkyl_{(C≤12)}, substituted alkyl_{(C≤12)}, cycloalkyl_{(C≤12)}, or substituted cycloalkyl_{(C≤12)}; or
a compound of the formula: 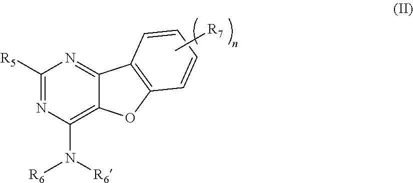
wherein:
R₅ is hydrogen or alkyl_{(C≤12)}, cycloalkyl_{(C≤12)}, aryl_{(C≤12)}, aralkyl_{(C≤12)}, or a substituted version of any of these four groups;
R₆ is hydrogen, alkyl_{(C≤12)}, substituted alkyl_{(C≤12)}, cycloalkyl_{(C≤12)}, or substituted cycloalkyl_{(C≤12)};
R₆′ is hydrogen or alkyl_{(C≤12)}, cycloalkyl_{(C≤12)}, aralkyl_{(C≤12)}, or a substituted version of these three groups; or —RₐOR_{b}, wherein Rₐ is alkanediyl_{(C≤8)} or substituted alkanediyl_{(C≤8)} and R_{b} is alkyl_{(C≤8)}, cycloalkyl_{(C≤8)}, or a substituted version of either group;
R₆ and R₆′ are taken together and are alkanediyl_{(C≤8)} or substituted alkanediyl_{(C≤8)};
R₇ is amino, cyano, halo, hydroxy, or nitro, or alkyl_{(C≤6)}, cycloalkyl_{(C≤6)}, acyl_{(C≤6)}, alkoxy_{(C≤6)}, acyloxy_{(C≤6)}, amido_{(C≤6)}, alkylamino_{(C≤6)}, dialkylamino_{(C≤6)}, alkylsulfonyl_{(C≤6)}, alkylsulfonylamino_{(C≤6)}, or a substituted version of these ten groups; and
n is 0, 1, 2, 3, or 4;
or a pharmaceutically acceptable salt thereof;
provided that the compound is not:

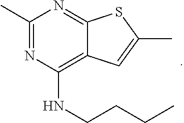 [SMILES: CCCCNc1nc(C)nc2sc(C)cc12]

91. The method of claim 90, wherein the compound is further defined as: 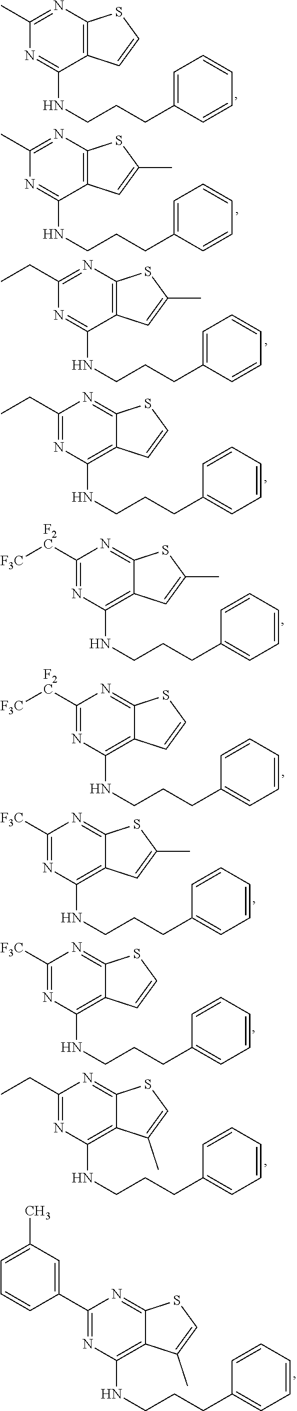 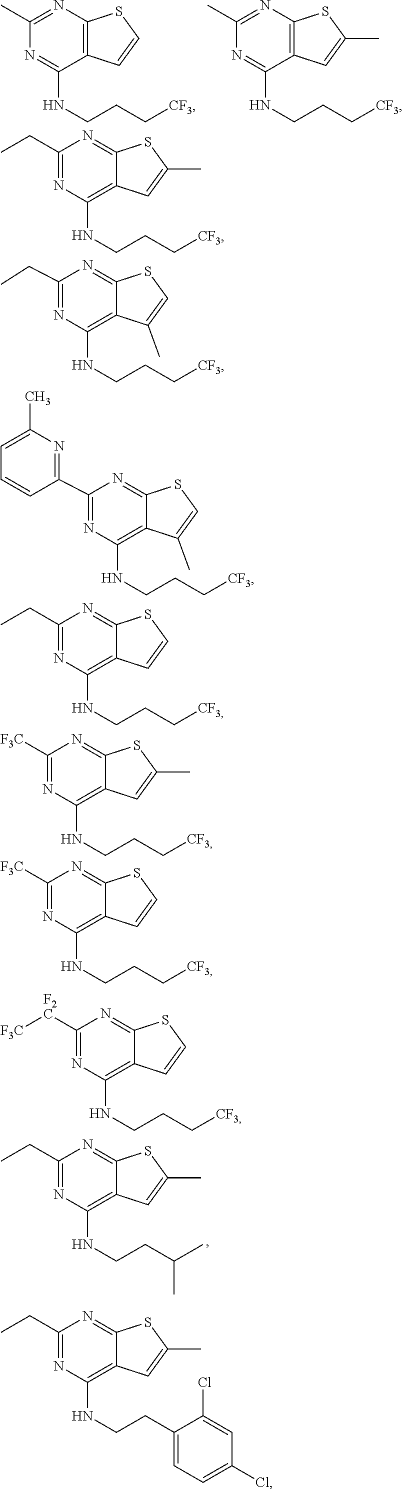 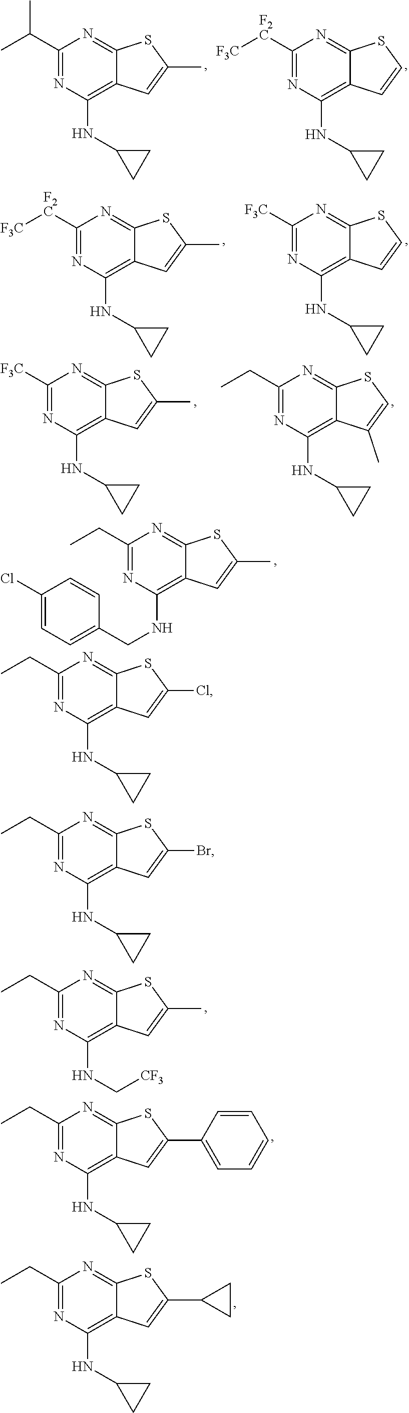 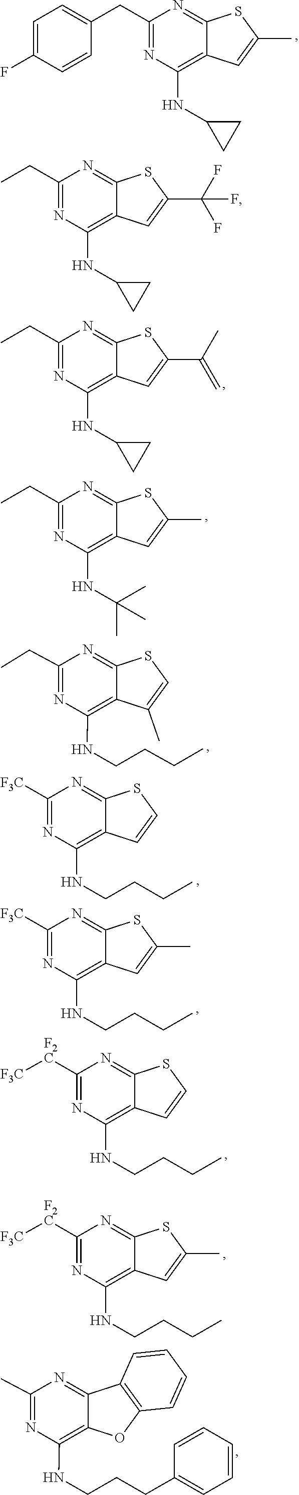 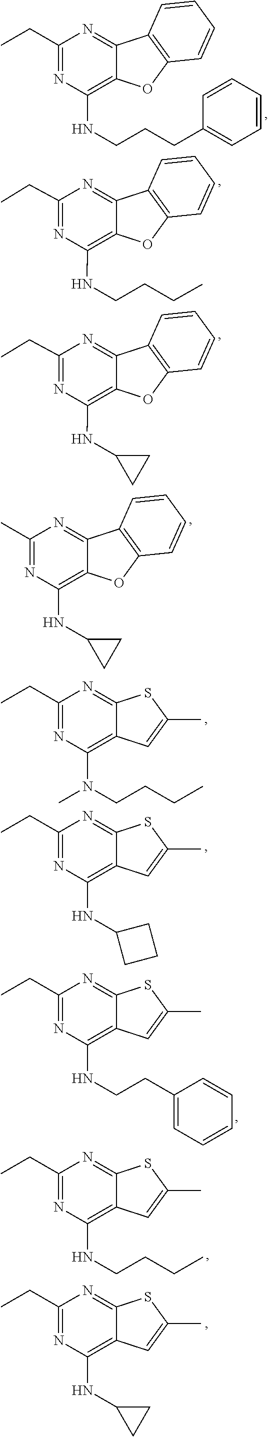 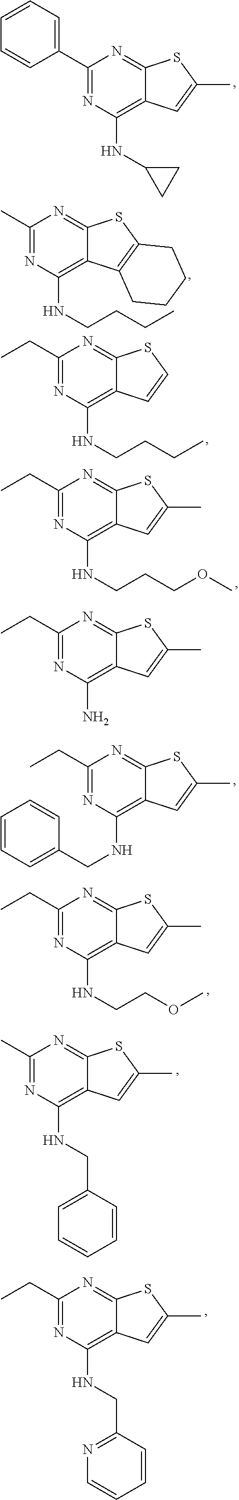 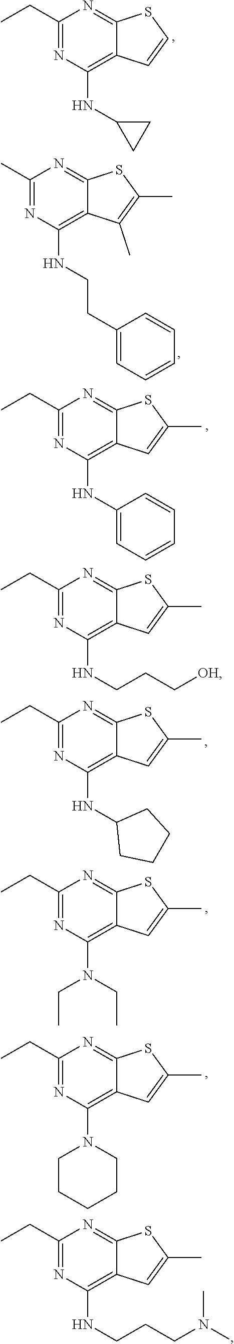 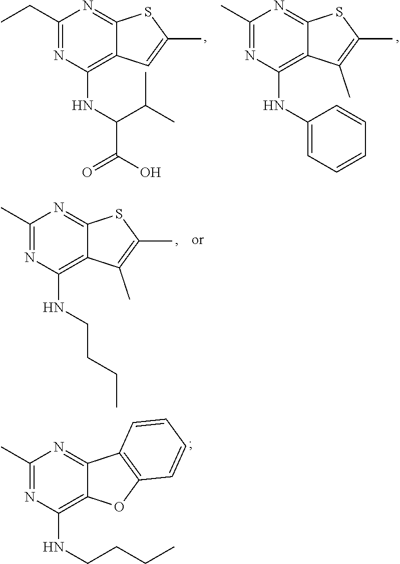
or a pharmaceutically acceptable salt thereof.

92. The method of either claim 90 or claim 91, wherein the method is sufficient to treat a Mycobacterium tuberculosis infection in a patient.
